(19) United States
(12) Patent Application Publication
(10) Pub. No.: US 2025/0249059 A1
(43) Pub. Date: Aug. 7, 2025

(54) PLANT BASED HONEY COMPOSITION

(71) Applicant: MELIBIO, INC., Oakland, CA (US)

(72) Inventors: Aaron M. SCHALLER, Oakland, CA (US); Darko MANDICH, Oakland, CA (US); Katherine UHL, Oakland, CA (US)

(21) Appl. No.: 19/012,569

(22) Filed: Jan. 7, 2025

Related U.S. Application Data
(63) Continuation of application No. PCT/US2023/069784, filed on Jul. 7, 2023.
(60) Provisional application No. 63/367,879, filed on Jul. 7, 2022.

(51) Int. Cl. A61K 36/185; A61K 31/194; A61K 33/00; A61K 36/07; A61K 36/074; A61K 36/28; A61K 36/42; A61K 36/48; A61K 36/53; A61K 36/54; A61K 36/63; A61K 36/73; A61K 36/74; A61K 36/752; A61K 36/82; A61K 36/87; A61K 36/889; A61K 36/9068; A61K 47/26

(52) U.S. Cl. CPC A61K 36/185; A61K 31/194; A61K 33/00; A61K 36/07; A61K 36/074; A61K 36/28; A61K 36/42; A61K 36/48; A61K 36/53; A61K 36/54; A61K 36/5775; A61K 36/63; A61K 36/73; A61K 36/742; A61K 36/752; A61K 36/82; A61K 36/87; A61K 36/889; A61K 36/9068; A61K 47/26

(57) ABSTRACT

Disclosed herein are a non-bee made honey formulations comprising fructose in an amount from about 20% to about 55% w/w; glucose in an amount from about 20% to about 40% w/w; one or organic acids in an amount from about 0.001% to about 5% w/w; water; and one of the following: one or more polyphenols in a concentration of at least 0.4 mM; or a total antioxidant capacity of at least 1,500 nM/uL Trolox Equivalents. In some embodiments, the total antioxidant capacity is at least that of natural bee-made clover honey. In some embodiments, the total antioxidant capacity exceeds that of natural bee-made clover honey.

### CLAIM OF PRIORITY

This application is a continuation of International Patent Application PCT/US2023/069784, filed on Jul. 7, 2023, entitled to the benefit of and claims priority to U.S. App. No. 63/367,879 filed on Jul. 7, 2022, entitled PLANT BASED HONEY COMPOSITION, each of which is incorporated by reference herein in its entirety.

### FIELD OF THE INVENTION

The present invention is generally in the field of non-bee made honey formulations.

### BACKGROUND

The population of honeybees in the U.S. has decreased by about 45% in 2021, a decrease which has been deemed “unsustainable,” and which places the various ecosystems dependent upon honeybees for pollination at risk. Honeybees are also currently the only method of production available for producing honey. Further, use of honeybees commercially for honey production poses a threat to native and wild bee populations—essential pollinators of many flowering plants and trees. There remains a need in the art for a replacement for natural honeys to reduce the dependency and burden placed upon bees as a result of honey production.

### SUMMARY OF THE INVENTION

There are a lack of available honey alternatives which closely approximate the taste, aroma, mouthfeel, texture, appearance, color, and overall flavor of natural honey. It would be desirable to provide a plant based alternative to natural honey, which can have different flavors and fragrances, to mimic the variations that occur naturally, and provide plant based honey compositions having known and quantitated amounts of polyphenols, antioxidants, and organic acids associated with specific health benefits, and flavor profiles. It would also be desirable to have a honey alternative product which does not contain any allergens, bee parts, pollens, other organic proteins which could serve as potential allergens to consumers of honey, or spores of botulin toxin which render honey unsuitable for consumption by infants. Alternative to natural honey also tend to lack desirable natural properties of natural honey (for example, honey's naturally high antioxidant capacity), and/or desirable natural ingredients (for example, polyphenols), and attempting to add back in artificial sources of such desirable natural ingredients in an effort to recreate the desired natural properties can lead to a product with qualities that fail to mimic that of natural honey, or which has an artificial or undesirable characteristic that make the product an unappealing alternative. There are thousands of possible ingredients which may be selected to attempt to achieve these properties, and minimal to no guidance provided as to the selection of effective components for such formulations that closely mimic that of natural honey as the have a similar appeal to consumers. For instance, existing formulations are often have a bitter or artificial taste, an off-putting texture or appearance, or other undesirable characteristics associated with such non-bee made formulations. Accordingly, there remains a need within the art for the disclosure of non-bee made honey formulations which mimic that of natural honey in a manner which is appealing to consumers, and which also have desirable natural properties of natural honey (for example, honey's naturally high antioxidant capacity), and/or desirable natural ingredients (for example, polyphenols).

Production of non-bee made honey products has been attempted by the inventors by utilizing a blend of sugars and organic acids which are naturally found in honey. However, the results included unsatisfactory products which failed to closely approximate bee-made clover honey in a manner which would be acceptable to a consumer. Bee-made honeys may contain a variety of compounds such as polyphenols, acids, vitamins, minerals, amino acids, enzymes, ash, among other compounds, which account for less than about 1% of the honey by mass, but which are critical to the flavor, texture, appearance, mouthfeel, and other properties of honey for which consumers purchase honey. Of these ingredients, the plant extracts, polyphenols and organic acids alone number in the hundreds or thousands of possible ingredients, and there is little to no guidance provided as to selection of the plant extracts, polyphenols and organic acids which may be used to closely approximate bee-made honey, or provide desirable natural properties of natural honey (for example, honey's naturally high antioxidant capacity). Further, the identity of the plant extracts, polyphenols and organic acids naturally found widely varies among the different types of honeys. Similarly, there are at least tens of thousands of possible combinations of these ingredients, and many different possible concentrations for each ingredient, and choosing particular plant extracts, polyphenols and organic acids sufficient to produce a non-bee made honey formulation which closely approximates bee-made honey remains a challenge. As such, disclosed herein are non-bee made honey formulations with unique combinations and concentrations of sugars, polyphenols, organic acids, and plant extracts which produce a non-bee made honey formulation closely approximating that of natural clover honey, and which have desirable natural properties of natural honey (for example, honey's naturally high antioxidant capacity).

Provided herein is a non-bee made honey formulation comprising: fructose in an amount from about 20% to about 55% w/w; glucose in an amount from about 20% to about 40% w/w; one or organic acids in an amount from about 0.0010% to about 5% w/w; water; and one of the following: one or more polyphenols in a concentration of at least 0.4 mM; or a total antioxidant capacity of at least 1,500 nM/uL Trolox Equivalents. In some embodiments, the total antioxidant capacity is at least that of natural bee-made clover honey. In some embodiments, the total antioxidant capacity exceeds that of natural bee-made clover honey. In some embodiments, the total antioxidant capacity exceeds that of natural bee-made clover honey by at least 2.5% wt. In some embodiments, the total antioxidant capacity is at least 2,500 nM/uL Trolox Equivalents. In some embodiments, the one or more polyphenols are present in a concentration at least 3× that of bee made honey. In some embodiments, the total polyphenols are present in a concentration of at least 0.4 mM. In some embodiments, the total polyphenols are present in a concentration from about 0.4 mM to about 2.0 mM. In some embodiments, the total polyphenols are present in a concentration of at least 0.7 mM. In some embodiments, the total polyphenols are present in a concentration of at least 0.4 mM catechin equivalent (CE). In some embodiments, the total polyphenols are present in a concentration from about 0.4 mM catechin equivalent (CE) to about 2.0 mM catechin equivalent (CE). In some embodiments, the total polyphenols are present in a concentration of at least 0.7 catechin equivalent (CE). In some embodiments, the non-bee made honey formulation comprises one or more extracts are selected from: Sea buckthorn, Chamomile, Red Clover, Jasmine, Lavender, Succinic acid, or Passionflower. In some embodiments, the non-bee made honey formulation comprises Sea buckthorn extract, and one or more extracts selected from: Chamomile, Red Clover, Jasmine, Lavender, Succinic acid, or Passionflower. In some embodiments, the non-bee made honey formulation comprises Sea buckthorn extract, and three or more extracts selected from: Chamomile, Red Clover, Jasmine, Lavender, Succinic acid, or Passionflower. The non-bee made honey composition of claim 1, further comprising Sea buckthorn extract, Chamomile extract, Red Clover, and Succinic acid. In some embodiments, the non-bee made honey formulation comprises one or more extracts comprising: Sea buckthorn, Reishi mushroom, Chaga mushroom, Green coffee bean, Ginger, Pu'erh tea, Matcha, Acai berry, Aronia berry, Hibiscus, Squash blossom, Chamomile, Cinnamon, Orange blossom, Green tea, Lemon or lemon juice, white tea, grape seed, or Shilajit (mineral powder). In some embodiments, the non-bee made honey formulation comprises at least two extracts selected from: Sea buckthorn, Reishi mushroom, Chaga mushroom, Green coffee bean, Ginger, Pu'erh tea, Matcha, Acai berry, Aronia berry, Hibiscus, Squash blossom, Chamomile, Cinnamon, Orange blossom, Green tea, Lemon, white tea, grape seed, or Shilajit (mineral powder). In some embodiments, the non-bee made honey formulation comprises at least three extracts that are selected from: Reishi mushroom, Chaga mushroom, Green coffee bean powder, Ginger powder, Pu'erh tea, Matcha powder, Acai berry, Aronia berry, Hibiscus flower, Squash blossom, Chamomile flower, Cinnamon, Orange blossom, Green tea, Lemon, or Shilajit (mineral powder). In some embodiments, the non-bee made honey formulation comprises Sea buckthorn extract and at least two extracts selected from: Reishi mushroom, Chaga mushroom, Green coffee bean, Ginger, Pu'erh tea, Matcha, Acai berry, Aronia berry, Hibiscus flower, Squash blossom, Chamomile flower, Cinnamon stick, Orange blossom, Green tea, Lemon or lemon juice, or Shilajit (mineral powder). In some embodiments, the non-bee made honey formulation comprises Sea buckthorn extract and at least three extracts selected from: Reishi mushroom powder, Chaga mushroom, Green coffee bean, Ginger, Pu'erh tea, Matcha, Acai berry, Aronia berry, Hibiscus flower, Squash blossom, Chamomile, Cinnamon stick, Orange blossom, Green tea, Lemon, or Shilajit (mineral powder). In some embodiments, the non-bee made honey comprises at least one ingredient shown in Table 5 wherein the at least one ingredient is optionally in the stated percentage of Table 5. In some embodiments, the non-bee made honey comprises ingredients shown in Table 6 wherein the at least one ingredient is optionally in the stated percentage of Table 6. In some embodiments, the non-bee made honey comprises ingredients shown in Table 7, wherein the at least one ingredient is optionally in the stated percentage of Table 7. In some embodiments, the non-bee made honey comprises ingredients shown in Table 8, wherein the at least one ingredient is optionally in the stated percentage of Table 8. In some embodiments, the non-bee made honey comprises protein in an amount less than 0.17% w/w. In some embodiments, the non-bee made honey comprises is substantially devoid of protein. In some embodiments, the non-bee made honey comprises one or more proanthocyanidins. In some embodiments, the one or more proanthocyanidins are present in an amount up to about 0.2 mg per 30 mL of the formulation. In some embodiments, the one or more proanthocyanidins are present in an amount up to about 0.02 mg per 30 mL of the formulation. In some embodiments, the one or more proanthocyanidins are present in an amount of at least 0.0001%. In some embodiments, the one or more proanthocyanidins are present in an amount of 0.0001% to about 0.02% w/w. In some embodiments, the one or more proanthocyanidins are present in an amount of 0.00010% to about 0.2% w/w. In some embodiments, the one or more proanthocyanidins are present in an amount of 0.00001% to about 0.001% w/w. In some embodiments, the one or more proanthocyanidins are present in an amount of 0.0001% to about 0.002% w/w. In some embodiments, the non-bee made honey comprises one or more proanthocyanidins in an amount from 0.0001% to about 0.1% w/w. In some embodiments, the non-bee made honey comprises one or more proanthocyanidins in an amount from about 5 mg to about 20 mg per 30 mL of the formulation. In some embodiments, the non-bee made honey comprises one or more proanthocyanidins in an amount from 0.0001% to about 0.001% w/w. In some embodiments, the non-bee made honey comprises one or more polyphenols including comprise kaempferol, quercetin, and isorhamnetin. In some embodiments, the non-bee made honey comprises sea buckthorn extract. In some embodiments, the non-bee made honey comprises sea buckthorn extract in an amount of from 0.01% to about 1% w/w of the formulation. In some embodiments, the non-bee made honey comprises sea buckthorn extract in an amount from 0.05% to about 0.5% w/w. In some embodiments, the non-bee made honey comprises sea buckthorn extract in an amount of about 0.1% w/w. In some embodiments, the one or more polyphenols comprise quercetin in an amount greater than 0.0015% w/w. In some embodiments, the quercetin is present in an amount from about 0.0015% to about 0.1% w/w. In some embodiments, the quercetin is present in an amount of about 0.003% to about 0.005% w/w. In some embodiments, the non-bee made honey formulation comprises one or more organic acids comprising gluconic acid in an amount from about 0.001% to about 0.5% w/w. In some embodiments, the non-bee made honey formulation comprises one or more organic acids comprising gluconic acid in an amount from about 0.05% to about 0.5% w/w. In some embodiments, the non-bee made honey formulation comprises one or more organic acids comprising gluconic acid in an amount of about 0.1% w/w. In some embodiments, the non-bee made honey formulation comprises one or more organic acids comprising succinic acid in an amount from about 0.001% to about 0.5% w/w. In some embodiments, the non-bee made honey formulation comprises one or more organic acids comprising succinic acid in an amount from about 0.05% to about 0.5% w/w. In some embodiments, the non-bee made honey formulation comprises one or more organic acids comprising succinic acid in an amount of about 0.01% w/w. In some embodiments, the non-bee made honey formulation comprises one or more organic acids comprising chlorogenic acid in an amount from about 0.001% to about 0.01% w/w. In some embodiments, the non-bee made honey formulation comprises one or more organic acids comprise chlorogenic acid in an amount of about 0.004% w/w. In some embodiments, the non-bee made honey formulation comprises one or more organic acids comprise gallic acid in an amount from about 0.001% to about 0.01% w/w. In some embodiments, the non-bee made honey formulation comprises one or more organic acids comprise gallic acid in an amount of about 0.005% w/w. In some embodiments, the non-bee made honey formulation comprises 50 milli-equivalents acid per 1000 g or less. In some embodiments, the non-bee made honey formulation comprises 10 milli-equivalents acid per 1000 g or less. In some embodiments, the non-bee made honey formulation comprises 1 milli-equivalent of acid per 1000 g or less. In some embodiments, the non-bee made honey formulation comprises one or more polyphenols comprising quercetin in an amount from about 0.005% to about 0.05% w/w. In some embodiments, the non-bee made honey formulation comprises one or more polyphenols comprising quercetin in an amount of about 0.01% w/w. In some embodiments, the non-bee made honey formulation comprises one or more polyphenols comprising chrysin in an amount from about 0.001% to about 0.01% w/w. In some embodiments, the non-bee made honey formulation comprises one or more polyphenols comprising apigenin in an amount from about 0.001% to about 0.5% w/w. In some embodiments, the non-bee made honey formulation comprises one or more polyphenols comprising apigenin in an amount of about 0.003% w/w. In some embodiments, the non-bee made honey formulation comprises one or more polyphenols comprising quercetin, galangin, hesperetin, chrysin, pinocembrin, naringenin, pinobanksin, luteolin, apigenin, genistein, catechin, epicatechin, isorhamnetic, myricetin, rutin, kaempferol, and derivatives. In some embodiments, the non-bee made honey formulation is substantially similar to bee made honey. In some embodiments, the non-bee made honey formulation comprises a viscosity substantially similar to bee made honey. In some embodiments, the non-bee made honey formulation comprises an appearance or color substantially similar to bee made honey. In some embodiments, the non-bee made honey formulation is a clover honey formulation, or approximates a characteristic of a clover honey formulation taste, an appearance, a texture, or a smell of bee made cover honey. In some embodiments, the natural clover honey which the non-bee made honey formulation is evaluated against is Signature Select clover honey. In some embodiments, the non-bee made honey formulation comprises no detectable protein by AOAC 2011.04 crude protein analysis. In some embodiments, the non-bee made honey formulation comprises less than 0.17% w/w protein by AOAC 2011.04 crude protein analysis. In some embodiments, the non-bee made honey formulation comprises no pollen detectable by AOAC 2011.04 crude protein analysis. In some embodiments, the non-bee made honey formulation comprises less than 0.17% w/w pollen detectable by AOAC 2011.04 crude protein analysis. In some embodiments, the non-bee made honey formulation comprises no pollen lipids detectable. In some embodiments, the non-bee made honey formulation comprises no allergens detectable by AOAC 2011.04 crude protein analysis. In some embodiments, the non-bee made honey formulation comprises no glycoproteins detectable by AOAC 2011.04 crude protein analysis. In some embodiments, the non-bee made honey formulation comprises glycoproteins in an amount less than 0.17% w/w. In some embodiments, the non-bee made honey formulation comprises a pH from about 3.0 to about 6.0. In some embodiments, the non-bee made honey formulation comprises a pH of about 3.72. In some embodiments, the non-bee made honey formulation comprises a water activity of about 0.4 to about 0.6. In some embodiments, the non-bee made honey formulation comprises a water activity of about 0.5. In some embodiments, the total antioxidant capacity exceeds that of natural bee-made clover honey by at least 2.5% wt. In some embodiments, the total antioxidant capacity is at least 2,500 nM/uL Trolox Equivalents. In some embodiments, the total antioxidant capacity is at least 1,000 nM/uL Trolox Equivalents. In some embodiments, the total antioxidant capacity is at least 500 nM/uL Trolox Equivalents. In some embodiments, the total antioxidant capacity is at least 100 nM/uL Trolox Equivalents. In some embodiments, the total antioxidant capacity is at least 3,000 nM/uL Trolox Equivalents. In some embodiments, the total antioxidant capacity is at least 3,500 nM/uL Trolox Equivalents. In some embodiments, the total antioxidant capacity is at least 4,000 nM/uL Trolox Equivalents. In some embodiments, the total antioxidant capacity is at least 4,500 nM/uL Trolox Equivalents. In some embodiments, the total antioxidant capacity is at least 5,000 nM/uL Trolox Equivalents. In some embodiments, the total antioxidant capacity is at least 5,500 nM/uL Trolox Equivalents. In some embodiments, the water in the non-bee made honey formulation comprises about 10% to about 40% w/w. In some embodiments, the water in the non-bee made honey formulation comprises about 25% to about 35% w/w. In some embodiments, the water in the non-bee made honey formulation comprises about 32% w/w. In some embodiments, the water in the non-bee made honey formulation comprises about 10% to about 20% w/w. In some embodiments, the water in the non-bee made honey formulation comprises about 15% w/w, wherein the non-bee made honey formulation is a low moisture formulation. In some embodiments, the non-bee made honey formulation comprises a water activity of about 0.40 to about 0.6. In some embodiments, the non-bee made honey formulation comprises a water activity of about 0.5. In some embodiments, the non-bee made honey formulation comprises a viscosity of about 14,000 cP to about 17,000 cP. In some embodiments, the non-bee made honey formulation comprises a viscosity of about 16,000 cP. In some embodiments, the non-bee made honey formulation comprises protein in an amount less than 0.16% w/w. In some embodiments, the non-bee made honey formulation comprises protein in an amount less than 0.15% w/w. In some embodiments, the non-bee made honey formulation comprises protein in an amount less than 0.14% w/w. In some embodiments, the non-bee made honey formulation comprises protein in an amount less than 0.13% w/w. In some embodiments, the non-bee made honey formulation comprises protein in an amount less than 0.12% w/w. In some embodiments, the non-bee made honey formulation comprises protein in an amount less than 0.11% w/w. In some embodiments, the non-bee made honey formulation comprises protein in an amount less than 0.1% w/w. In some embodiments, the non-bee made honey formulation comprises protein in an amount less than 0.9% w/w. In some embodiments, the non-bee made honey formulation comprises protein in an amount less than 0.8% w/w. In some embodiments, the non-bee made honey formulation comprises protein in an amount less than 0.7% w/w. In some embodiments, the non-bee made honey formulation comprises protein in an amount less than 0.6% w/w. In some embodiments, the non-bee made honey formulation comprises protein in an amount less than 0.5% w/w. In some embodiments, the non-bee made honey formulation comprises protein in an amount less than 0.4% w/w. In some embodiments, the non-bee made honey formulation comprises protein in an amount less than 0.3% w/w. In some embodiments, the non-bee made honey formulation comprises protein in an amount less than 0.2% w/w. In some embodiments, the non-bee made honey formulation comprises protein in an amount less than 0.1% w/w. In some embodiments, the one or more polyphenols are present in an amount of about 0.1% w/w. In some embodiments, the one or more polyphenols are present in an amount of about 0.2% w/w. In some embodiments, the one or more polyphenols are present in an amount of about 0.3% w/w. In some embodiments, the one or more polyphenols are present in an amount of about 0.4% w/w. In some embodiments, the one or more polyphenols are present in an amount of about 0.5% w/w. In some embodiments, the one or more polyphenols are present in an amount of about 0.6% w/w. In some embodiments, the one or more polyphenols are present in an amount of about 0.7% w/w. In some embodiments, the one or more polyphenols are present in an amount of about 0.8% w/w. In some embodiments, the one or more polyphenols are present in an amount of about 0.9% w/w. In some embodiments, the one or more polyphenols are present in an amount of about 1% w/w. In some embodiments, the one or more polyphenols are present in an amount of about 1.1% w/w. In some embodiments, the one or more polyphenols are present in an amount of about 1.2% w/w. In some embodiments, the one or more polyphenols are present in an amount of about 1.3% w/w. In some embodiments, the one or more polyphenols are present in an amount of about 1.4% w/w. In some embodiments, the one or more polyphenols are present in an amount of about 1.5% w/w. In some embodiments, the one or more polyphenols are present in an amount of about 1.6% w/w. In some embodiments, the one or more polyphenols are present in an amount of about 1.7% w/w. In some embodiments, the one or more polyphenols are present in an amount of about 1.8% w/w. In some embodiments, the one or more polyphenols are present in an amount of about 1.9% w/w. In some embodiments, the one or more polyphenols are present in an amount of about 2% w/w. In some embodiments, the one or more polyphenols are present in an amount of about 2.1% w/w. In some embodiments, the one or more polyphenols are present in an amount of about 2.2% w/w. In some embodiments, the one or more polyphenols are present in an amount of about 2.3% w/w. In some embodiments, the one or more polyphenols are present in an amount of about 2.4% w/w. In some embodiments, the one or more polyphenols are present in an amount of about 2.5% w/w. In some embodiments, the one or more polyphenols are present in an amount of about 2.6% w/w. In some embodiments, the one or more polyphenols are present in an amount of about 2.7% w/w. In some embodiments, the one or more polyphenols are present in an amount of about 2.8% w/w. In some embodiments, the one or more polyphenols are present in an amount of about 2.9% w/w. In some embodiments, the one or more polyphenols are present in an amount of about 3% w/w. In some embodiments, the one or more polyphenols are present in an amount of about 3.2% w/w. In some embodiments, the one or more polyphenols are present in an amount of about 3.4% w/w. In some embodiments, the one or more polyphenols are present in an amount of about 3.6% w/w. In some embodiments, the one or more polyphenols are present in an amount of about 3.8% w/w. In some embodiments, the one or more polyphenols are present in an amount of about 4% w/w.

In another aspect, provided herein is a method of producing the non-bee made honey formulation of any of the preceding claims, comprising: mixing glucose and fructose in water under elevated temperature to form a first solution; mixing one or more organic acids, and one or more polyphenols in water under elevated temperature to form a second solution; and mixing the first solution and the second solution to form a third solution; and dehydrating the third solution at elevated temperature while mixing to reduce the water content of the solution.

### BRIEF DESCRIPTION OF DRAWINGS

FIG. 1 shows results of a sensory panel comparing the various sensory properties of the non-bee made honey formulations disclosed herein.

FIG. 2 shows results of a sensory panel comparing the likeability of the non-bee made honey formulations disclosed herein.

FIG. 3 shows results of a catechin equivalent assay quantifying the total phenolic content of the non-bee made honey formulations disclosed herein.

FIG. 4 shows Total Antioxidant Capacity in nM/uL Trolox Equivalents for an exemplary non-bee made honey formulation disclosed herein compared to clover bee-made honey (n=3).

FIG. 5 shows Total Phenolic Content in mM Catechin equivalents an exemplary non-bee made honey formulation disclosed herein compared to clover bee-made honey (n=2).

FIG. 6 provides images illustrating the general appearance and color of a natural bee made honey as compared to an exemplary non-bee made honey formulations disclosed herein.

FIG. 7 provides images illustrating the appearance, viscosity, stiffness, and/or cohesiveness of a natural bee made honey as compared to an exemplary non-bee made honey formulations disclosed herein.

### DETAILED DESCRIPTION OF THE INVENTION

There are at least 300 different types of honey, and each of them has a different appearance, flavor, texture, and fragrance. One popular type of honey is clover honey, known for its sweet taste, flowery flavor, and pleasant mild taste. Clover honey is the most commonly sold and consumed honey in the United States, and is the honey which is most commonly associated with honey products. Clover honey in many cases may be about 80% sugars, 17% water, and 3% other components, including a hodgepodge of trace compounds (e.g., polyphenols, acids, vitamins, minerals, amino acids, enzymes, ash, nectar, etc.) that are critical to the honey varied flavors and colors as well as the nutritional value of honey. Polyphenols in particular play a role in the antioxidant properties of honey. Polyphenol antioxidant activity has been associated with several health benefits in humans and mammals due to their ability to scavenge free radicals. Free radicals, which can cause oxidative damage, disrupt cellular organelles, and contribute to aging are thus preferably removed from the body as soon as possible. A high antioxidant content in food products can contribute to the neutralization of free radicals, making honey a valuable food product. In particular, polyphenols are bioavailable forms of antioxidants, making their presence even more valuable. Clover honey is commonly referred to as “honey” because of its significant popularity among consumers, and for its sweet and mild flavor, with mild floral notes, and lack of significant aftertaste. Preparing a clover honey composition which closely approximates the flavor, texture, and other properties of honey, for example clover honey, remains a challenge as it is unknown precisely what trace compounds, and what quantities thereof, are responsible for the unique flavor, texture, aroma, mouthfeel, appearance, and other properties of clover honey. The population of honeybees in the U.S. has decreased 40% in the winter of 2018 to 2019, and the annual rate loss for the 2019 to 2020 winter was also 40%; declines that experts agree is “unsustainable.” There remains a need in the art for a replacement for natural honeys to reduce the dependency and burden placed upon bees as a result of honey production.

Honeybees labor to transform collected flower nectar into a highly concentrated sugary sweet substance broadly referred to as “honey.” The action of enzymes produced in the saliva and honey stomach of the bees act on collected flower nectar to transform it into a storable food source, meant to sustain honeybee colonies during months when flowers are not in bloom. Nectar is foraged through the proboscis of the female worker bee and is stored in a specialized organ called the “honey stomach.” Immediately upon storage, enzymes secreted in the honey stomach begin to transform the fluid. This occurs primarily through the actions of invertase and glucose oxidase. Invertase is responsible for the inversion of the disaccharide sucrose into the monosaccharides fructose and glucose. Glucose oxidase also works to consume the newly inverted glucose to produce gluconic acid and hydrogen peroxide. The action of these enzymes is a hallmark of bee-made honey and provides several benefits. The monosaccharides fructose and glucose provide a solution of sugars that is easier to digest, and lasts for a longer period without crystallization occurring, two aspects that are important for food quality. Furthermore, gluconic acid is a weak acid, and its presence in the final honey product lowers the pH of the honey to a level that prohibits the growth of microbiological organisms such as yeast that would otherwise ferment the honey. However, the correct selection and relative proportions of sugars in a honey substitute product, the correct selection and relative proportions of organic acids, and presence of lack of enzymes in a final non-bee made honey product remain difficult to predict when preparing a product which will closely approximate the taste, aroma, appearance, mouthfeel, viscosity, and color of natural honey.

Another important process occurs that is a hallmark of the transformation of nectar to honey via the bee is the passing of nectar through regurgitation between bees. The purpose of this process is thought to be two-fold. First, passing nectar from bee to bee ensures that enzymes are added to the mixture during each handoff, meaning that the enzymes are working constantly and being replenished to complete the work of transforming the nectar. Second, this process aids in dehydration of the honey as water from the original nectar is consumed during each handoff. Slowly but surely the water content drops from nearly 80% down below 20%. At this time, after regurgitating the honey between approximately 20 bees, the viscosity is too high to transfer and the honey is now stored and sealed in the honeycomb for consumption later. This low water content also helps to keep the honey free from microbial activity while it is being stored. The bee, in the context of transforming nectar into honey, acts as a medium to transform a relatively fluid and less complicated solution, nectar, into a viscous concentration of sugars and acids that is a more dependable food source.

While the incredible qualities of this food product cannot be understated, it may contain several ingredients or qualities, that can be unwanted by the consumer. For example, honey in its raw form is known to be dangerous to infants. This is because it can contain the pathogenic spores of Clostridium botulinum that the infant immune system is poorly equipped to combat. Furthermore, honey often contains apitoxin or bee venom, introduced by way of the regurgitation of honey from bee to bee before storage. Bee venom can be a harmful allergen, even when consumed, making it a potentially dangerous product for many. Honey may contain pollen or bee pollen which can be an allergen. Bee parts from old bees, or bees killed during the honey harvest, often make their way into the final product as well. These bee parts are not only unsavory to many, but simply unacceptable to the growing population of consumers that follow a plant-based or vegan diet regime. Bee honey is also known to contain some residual amounts of pollen or other particles which have allergenic effects, such that the honey itself can be an allergen. Lastly, dangerous chemicals and antibiotics can often find their way into bee-made honey due to the wide range that bees travel to forage (up to 5 km from their hive), and due to spraying of hives with antibiotic solutions to control pathogens. Even if a beekeeper does not spray their hives or their own plants nearest the hive, bees do not forage in a controlled environment. They can fly vast distances to collect nectar from neighboring orchards or fields that have been chemically treated, and thereby return with tainted honey or pollen that is introduced to the hive and the colony, and ultimately the honey.

The consumer may find several “honey-like” food products on retail shelves. These include colored sugar syrups, reduced fruit sugar syrups, malt extract syrups, brown rice syrups, and many more. However, most honey alternatives are made of fructose or sucrose with sweeteners such as corn syrup, and do not have the consistency nor flavor of honey. These products are often marketed as “honey alternatives” due to their accounting for only the most obvious characteristics of honey: color (which has a wide range), relative viscosity (e.g., a thickened liquid), and sweetness. The other common traits found in honey alternatives include the following: sugar content is predominantly sucrose, they contain ingredients that are not found in bee-made honey, and they overwhelmingly lack depth in terms of ingredients beneficial to human health. These so-called honey alternatives lack some of the important components of honey that make it a “superfood”, components that are imparted by the bees directly from plants, such as polyphenols and organic acids, many of which have gained considerable recognition in recent years as their health effects have become highly touted by the medical community.

As such, there is a growing need for a way to create a honey product that contains the most beneficial aspects of honey (proper sugar selection and balance, complex and pleasing flavor profile and aroma, antioxidant and bioactive compounds, organic acids and plant compounds such as polyphenols and flavonoids), while leaving out the harmful and unwanted ingredients in honey (antibiotics, pesticides, bee-venom, pathogenic spores, pollen, allergens, bee parts, etc.). However, while there are numerous reports of honey having therapeutic properties, it is difficult to identify which compounds may have specific activities, due to variations in composition and in amounts. In particular, the phenolic content of a particular type of honey is not always known, and it can be difficult to determine whether any observed effects on human health may be associated with the consumption of honey or its constituents. Similarly, exactly what role the various organic acids, and plant compounds such as polyphenols and flavonoids play in creating the unique appearance, texture, mouthfeel, flavor, and aroma of honey is also unknown and must be determined via extensive experimentation to produce a high quality non-bee made honey substitute product.

Honey is generally thought of as a viscous sweet liquid formed from flower nectar through collection and partial digestion by bees. However, in its essence, honey is primarily a mixture of sugars, chemicals, and aromatic compounds, all of which can be found elsewhere in nature primarily in the plant kingdom. Although the bee is an efficient worker and vehicle for honey production, other means exist by which to create honey in its final form. Formulas have been created as disclosed herein composed of simple sugars, acids, and several minor components which replicates a specific pH range, viscosity, water content, fragrance and flavor to match that of honey. The non-bee made honey formulations disclosed herein may not include pollen, proteins, or allergens. The plant based non-allergenic honey described herein addresses this need and includes ingredients that make honey real in addition to compounds found in honey known to promote human health that may not have been previously appreciated by the consumer. Furthermore, this plant-based honey is created in a controlled setting to prevent the inclusion of any pathogenic spores or microorganisms, and is guaranteed free of bee parts and bee venom due to bees not being used in the process, and does not contain harmful amounts of pesticides or antibiotics due to the sourcing of high-quality plant-based ingredients. The product is thereby effectively plant-based, non-allergenic, and infant-safe.

The plant-based or non-bee made honey is made by bypassing the bee in method of production by reconstituting ingredients found in the fully transformed honey product (final sugars, acids, polyphenols etc.), including ingredients found in traditionally made bee honey, including a blend of organic acids which may approximate a favor profile found in bee-made honey, and including other plant compounds such as polyphenols and flavonoids to approximate the appearance, texture, flavor, mouthfeel, and aroma of bee made honey, taking into account the incredible diversity of health-promoting micronutrients imparted to honey directly from plants in the form of polyphenols. In some embodiments, the plant-based or non-bee made honey disclosed herein may be intended to approximate bee-made clover honey, and the plant-based or non-bee made honey may be a clover honey substitute with a sweet, mild flavor, with light floral notes, and minimal aftertaste.

### I Definitions

Honey is defined by general usage and by the US Food and Drug Administration (“FDA”) as a thick, sweet, syrupy substance that bees make as food from the nectar of plants or secretions of living parts of plants and store in honeycombs. A product consisting of honey and a sweetener cannot be labeled with the common or usual name “honey” because “[t]he common or usual name of a food . . . shall accurately identify or describe . . . the basic nature of the food or its characterizing properties or ingredients” (21 CFR 102.5(a)). For honey substitutes, the label must include the usual name of each ingredient. In the case of most honey alternatives which contain some honey, the ingredient statement would show “honey” and the common or usual name of the sweetener (e.g., “sugar,” “corn syrup”), in descending order of predominance by weight (see section 403(i) of the FD&C Act and 21 CFR 101.4(a)(1)).

Honey substitutes are typically understood to consist of sugar(s), syrups and/or sweeteners, and few, if any, other ingredients.

As used herein, a plant based honey and non-bee made honey has a similar sugar content and acidity as bee made honey and includes small molecular weight compounds (2 kD or less), typically carboxylic and other organic acids, and polyphenolic compounds, that are used to impart flavor and/or fragrance.

As used herein “Generally recognized as safe (GRAS)” is a United States Food and Drug Administration (FDA) designation that a chemical or substance added to food is considered safe by experts and is listed in 21 CFR.

As used herein, an organic acid is an organic compound with acidic properties. The most common organic acids are the carboxylic acids, whose acidity is associated with their carboxyl group (—COOH). Sulfonic acids, containing the group —SO₂OH, are relatively stronger acids. Alcohols, with hydroxy groups (—OH), can act as acids but are usually very weak. The relative stability of the conjugate base of the acid determines its acidity. GRAS organic acids are preferred.

As used herein, a food acid is a GRAS acid suitable to add to a food. Representative food acids include gluconic, succinic, citric, acetic, formic, p-coumaric, chlorogenic, gallic, syringic, caffeic, oxalic, butyric, lactic, ferulic, ellagic, vanillic, kojic, protocatechuic, malic, and cinnamic acid.

As used herein, a polyphenolic is a compound containing more than one phenolic hydroxyl group derived from a plant. Useful polyphenolic compounds are GRAS compounds that can be added as an antioxidant, acidity modifier, flavoring or fragrance. Polyphenolic compounds are typically obtained from foods such as coffee, tea, cocoa, and grape seeds or grape must, and are typically extracted from seeds, roots, leaves and fruits.

As used herein, the term “about” a number refers to that number plus or minus 10% of that number. The term “about” a range refers to that range minus 10% of its lowest value and plus 10% of its greatest value.

As used herein “catechin equivalents” refers to the units of total phenolics concentration found in a sample as measured by a catechin equivalent assay. Phenolic compounds couple with diazonium salts under alkaline conditions to form a stable diazo chromophore, detectable by absorbance at 480 nm. The assay may not be affected by non-phenolic reducing substances such as sulfites, reducing sugars, or ascorbic acid. The assay may high-throughput adaptable and can detect concentrations of phenolic compounds as low as 0.02 mM catechin equivalents (CEs) from a variety of plant and food-based samples.

### II Compositions

Ingredients derived from plants are used to reconstitute the ingredient profile of bee-made honey to form what is effectively plant-based or plant-derived honey, the defining characteristic being that it does not employ the use of bees in its production, does not include any ingredients not normally found in honey, and preferably does not contain any protein or allergens. In some embodiments, the non-bee made honey formulation is substantially similar to bee made honey. In some embodiments, the non-bee made honey formulation comprises a viscosity substantially similar to bee made honey. In some embodiments, the non-bee made honey formulation comprises an appearance or color substantially similar to bee made honey. In some embodiments, the non-bee made honey formulation is a clover honey formulation. In some embodiments, the natural clover honey which the non-bee made honey formulation is evaluated against is Signature Select clover honey. In some embodiments, the natural clover honey which the non-bee made honey formulation is evaluated against is a regional honey variety. In some embodiments, the natural clover honey which the non-bee made honey formulation is evaluated against is Manuka honey. In some embodiments, the natural clover honey which the non-bee made honey formulation is evaluated against is European honey. In some embodiments, the natural clover honey which the non-bee made honey formulation is evaluated against is North American honey. In some embodiments, the natural clover honey which the non-bee made honey formulation is evaluated against is South American honey. In some embodiments, the natural clover honey which the non-bee made honey formulation is evaluated against is Asian honey. In some embodiments, the non-bee made honey formulation comprises no detectable protein by AOAC 2011.04 crude protein analysis. In some embodiments, the non-bee made honey formulation comprises less than 0.17% w/w protein by AOAC 2011.04 crude protein analysis. In some embodiments, the non-bee made honey formulation comprises no pollen detectable by AOAC 2011.04 crude protein analysis. In some embodiments, the non-bee made honey formulation comprises less than 0.17% w/w pollen detectable by AOAC 2011.04 crude protein analysis. In some embodiments, the non-bee made honey formulation comprises no detectable pollen lipids. In some embodiments, the non-bee made honey formulation comprises no allergens detectable by AOAC 2011.04 crude protein analysis. In some embodiments, the non-bee made honey formulation comprises no glycoproteins detectable by AOAC 2011.04 crude protein analysis. In some embodiments, the non-bee made honey formulation comprises glycoproteins in an amount less than 0.17% w/w. In some embodiments, the non-bee made honey formulation comprises glycoproteins in an amount less than 0.01% w/w. In some embodiments, the non-bee made honey formulation comprises glycoproteins in an amount less than 0.001% w/w.

Provided herein is a non-bee made honey formulation comprising: fructose in an amount from about 20% to about 55% w/w; glucose in an amount from about 20% to about 40% w/w; one or organic acids in an amount from about 0.0010% to about 5% w/w; water; and one of the following: one or more polyphenols in an amount from 0.001% to about 5% w/w, wherein the total polyphenols in the formulation are present in a concentration of at least 0.4 mM; or a total antioxidant capacity of at least 1,500 nM/uL Trolox Equivalents. In some embodiments, the fructose is in an amount from about 20% to about 50% w/w. In some embodiments, the fructose is in an amount from about 25% to about 50% w/w. In some embodiments, the fructose is in an amount from about 30% to about 50% w/w. In some embodiments, the fructose is in an amount from about 20% to about 45% w/w. In some embodiments, the fructose is in an amount from about 25% to about 45% w/w. In some embodiments, the fructose is in an amount from about 30% to about 45% w/w. In some embodiments, the fructose is in an amount from about 35% to about 45% w/w. In some embodiments, the percentage of fructose in the non-bee made honey composition is between about 20 and about 45% w/w. In some embodiments, the percentage of fructose in the non-bee made honey composition is between about 25 and 55% w/w. In some embodiments, the percentage of fructose in the non-bee made honey composition is between about 30 and 55% w/w. In some embodiments, the percentage of fructose in the non-bee made honey composition is between about 25 and 55% w/w. In some embodiments, the percentage of fructose in the non-bee made honey composition is between about 35 and 45% w/w. In some embodiments, the percentage of fructose in the non-bee made honey composition is between about 35 and 55% w/w. In some embodiments, the percentage of fructose in non-bee made honey composition is about 39%. In some embodiments, the glucose is in an amount from about 30% to about 45% w/w. In some embodiments, the amount of glucose is between about 20 and about 40% w/w. In some embodiments, the amount of glucose is between about 25 and about 40% w/w. In some embodiments, the amount of glucose is between about 30 and about 40% w/w. In some embodiments, the amount of glucose is between about 20 and about 35% w/w. In some embodiments, the amount of glucose is between about 25 and about 35% w/w. In some embodiments, the amount of glucose is between about 20 and about 35% w/w. In some embodiments, the amount of glucose is about 28%.

In some embodiments, the one or more organic acids comprises at least 3 organic acids. In some embodiments, the one or more organic acids comprises at least 4 organic acids In some embodiments, the one or more organic acids comprises at least 3 organic acids selected from: gluconic acid, succinic acid, chlorogenic acid, or gallic acid.

In some embodiments, the one or organic acids comprise gluconic acid in an amount from about 0.05% to about 0.5% w/w. In some embodiments, the one or organic acids comprise gluconic acid in an amount of about 0.10% w/w. In some embodiments, the one or organic acids comprise succinic acid in an amount from about 0.001% to about 0.5% w/w. In some embodiments, the one or organic acids comprise succinic acid in an amount from about 0.05% to about 0.5% w/w. In some embodiments, the one or organic acids comprise succinic acid in an amount of about 0.01% w/w. In some embodiments, the one or organic acids comprise chlorogenic acid in an amount from about 0.001% to about 0.01% w/w. In some embodiments, the one or organic acids comprise chlorogenic acid in an amount of about 0.004% w/w. In some embodiments, the one or organic acids comprise gallic acid in an amount from about 0.001% to about 0.01% w/w. In some embodiments, the one or organic acids comprise gallic acid in an amount of about 0.005% w/w. In some embodiments, the non-bee made honey formulation contains 50 milli-equivalents acid per 1000 g or less. In some embodiments, the one or more polyphenols are present in a concentration at least 3× that of bee made honey. In some embodiments, the one or more polyphenols are present in a concentration at least 2× that of bee made honey. In some embodiments, the one or more polyphenols are present in a concentration about the same as that of bee made honey. In some embodiments, the one or more polyphenols are present in a concentration less than that of bee made honey.

In some embodiments, the total polyphenols are present in a concentration of at least 0.4 mM. In some embodiments, the total polyphenols are present in a concentration of at least 0.01 mM. In some embodiments, the total polyphenols are present in a concentration of at least 0.1 mM. In some embodiments, the total polyphenols are present in a concentration of at least 1.0 mM. In some embodiments, the total polyphenols are present in a concentration from about 0.4 mM to about 2.0 mM. In some embodiments, the total polyphenols are present in a concentration from about 0.04 mM to about 2.0 mM. In some embodiments, the total polyphenols are present in a concentration from about 0.004 mM to about 2.0 mM. In some embodiments, the total polyphenols are present in a concentration from about 0.004 mM to about 0.4 mM. In some embodiments, the total polyphenols are present in a concentration from about 0.004 mM to about 0.4 mM. In some embodiments, the total polyphenols are present in a concentration from about 0.0004 mM to about 0.4 mM. In some embodiments, the total polyphenols are present in a concentration from about 0.004 mM to about 4.0 mM. In some embodiments, the total polyphenols are present in a concentration of at least 0.7 mM. In some embodiments, the total polyphenols are present in a concentration of at least 0.4 mM catechin equivalent (CE). In some embodiments, the total polyphenols are present in a concentration of at least 1.0 mM catechin equivalent (CE). In some embodiments, the total polyphenols are present in a concentration of at least 0.04 mM catechin equivalent (CE). In some embodiments, the total polyphenols are present in a concentration from about 0.4 mM catechin equivalent (CE) to about 2.0 mM catechin equivalent (CE). In some embodiments, the total polyphenols are present in a concentration from about 0.04 mM catechin equivalent (CE) to about 2.0 mM catechin equivalent (CE). In some embodiments, the total polyphenols are present in a concentration from about 0.004 mM catechin equivalent (CE) to about 2.0 mM catechin equivalent (CE). In some embodiments, the total polyphenols are present in a concentration from about 4.0 mM catechin equivalent (CE) to about 20.0 mM catechin equivalent (CE). In some embodiments, the total polyphenols are present in a concentration from about 0.4 mM catechin equivalent (CE) to about 20.0 mM catechin equivalent (CE). In some embodiments, the total polyphenols are present in a concentration from about 0.04 mM catechin equivalent (CE) to about 20.0 mM catechin equivalent (CE). In some embodiments, the total polyphenols are present in a concentration of at least 0.7 catechin equivalent (CE). In some embodiments, the total polyphenols are present in a concentration of at least 1.0 catechin equivalent (CE). In some embodiments, the total polyphenols are present in a concentration of at least 2.0 catechin equivalent (CE). In some embodiments, the total antioxidant capacity is at least that of natural bee-made clover honey. In some embodiments, the total antioxidant capacity exceeds that of natural bee-made clover honey. In some embodiments, the total antioxidant capacity exceeds that of natural bee-made clover honey by a factor of 2. In some embodiments, the total antioxidant capacity exceeds that of natural bee-made clover honey by a factor of 3. In some embodiments, the total antioxidant capacity exceeds that of natural bee-made clover honey by a factor of 5. In some embodiments, the total antioxidant capacity exceeds that of natural bee-made clover honey by a factor of 10. In some embodiments, the total antioxidant capacity exceeds that of natural bee-made clover honey by a factor of 100.

In some embodiments, the total antioxidant capacity exceeds that of natural bee-made clover honey by at least 2.5% wt. In some embodiments, the total antioxidant capacity is at least 2,500 nM/uL Trolox Equivalents. In some embodiments, the total antioxidant capacity is at least 1,000 nM/uL Trolox Equivalents. In some embodiments, the total antioxidant capacity is at least 500 nM/uL Trolox Equivalents. In some embodiments, the total antioxidant capacity is at least 100 nM/uL Trolox Equivalents. In some embodiments, the total antioxidant capacity is at least 3,000 nM/uL Trolox Equivalents. In some embodiments, the total antioxidant capacity is at least 3,500 nM/uL Trolox Equivalents. In some embodiments, the total antioxidant capacity is at least 4,000 nM/uL Trolox Equivalents. In some embodiments, the total antioxidant capacity is at least 4,500 nM/uL Trolox Equivalents. In some embodiments, the total antioxidant capacity is at least 5,000 nM/uL Trolox Equivalents. In some embodiments, the total antioxidant capacity is at least 5,500 nM/uL Trolox Equivalents.

In some embodiments, the non-bee made honey comprises protein in an amount less than 0.17% w/w. In some embodiments, the non-bee made honey comprises protein in an amount less than 0.01% w/w. In some embodiments, the non-bee made honey comprises is substantially devoid of protein.

### A. Ingredients

A first mixture component of the formulation may comprise a set of ingredients. Some of these ingredients may be present in most natural bee-made honeys and should therefore also be present in plant-based non-bee honeys. These base ingredients may include sugars, a selection of organic acids and/or a selection of polyphenolic compounds.

To match the composition of bee-made honey, the plant honey formulation may be adjusted to comparable sugar and acid content, pH range, and water content. The formulation may be non-specific with respect to particular honey type (e.g., clover honey), and multifunctional. The formulation aims to achieve several attributes that are expected of all honeys, for example, approximate: pH, viscosity/density, water activity, sweetness, and acidity.

### Sugars

The primary composition of the first solution is sugars and optionally acids. In some embodiments, glucose, fructose, and potentially sucrose and maltose make up the sweet fraction of the first solution. In some embodiments, glucose and fructose may comprise the sugars in the non-bee made honey formulation. Acids, which can be few to many, balance sweetness with their acidity and by lowering the overall pH of the formulation, which in turn contributes to the flavor profile, aroma, texture, and antibacterial nature of the non-bee made honey formulation.

In some embodiments, the percentage of fructose in the non-bee made honey composition is between about 20 and about 55% w/w. In some embodiments, the percentage of fructose in the non-bee made honey composition is between about 30 and 40% w/w. In some embodiments, the percentage of fructose in the non-bee made honey composition is between about 30 and 55% w/w. In some embodiments, the percentage of fructose in the non-bee made honey composition is between about 25 and 55% w/w. In some embodiments, the percentage of fructose in the non-bee made honey composition is between about 35 and 45% w/w. In some embodiments, the percentage of fructose in the non-bee made honey composition is between about 35 and 55% w/w. In some embodiments, the percentage of fructose in non-bee made honey composition is about 39%.

In some embodiments, the percentage of glucose in the final composition is between about 20 and about 40% w/w. In some embodiments, the percentage of glucose in the non-bee made honey composition is between about 25 and about 40% w/w. In some embodiments, the percentage of glucose in the non-bee made honey composition is between about 30 and about 40% w/w. In some embodiments, the percentage of glucose in the non-bee made honey composition is between about 30 and about 45% w/w. In some embodiments, the percentage of glucose in the non-bee made honey composition is about 28%.

In some embodiments, the percentage of fructose in the non-bee made honey composition is between about 20 and about 55% w/w. In some embodiments, the percentage of fructose in the plant-based honey composition is between about 30 and 40% w/w. In some embodiments, the percentage of fructose in the non-bee made honey composition is between about 30 and 55% w/w. In some embodiments, the percentage of fructose in the non-bee made honey composition is between about 25 and 55% w/w. In some embodiments, the percentage of fructose in the non-bee made honey composition is between about 35 and 45% w/w. In some embodiments, the percentage of fructose in the non-bee made honey composition is between about 35 and 55% w/w. In some embodiments, the percentage of fructose in non-bee made honey composition is about 39%.

In some embodiments, the percentage of glucose in the final non-bee made honey composition is between about 20 and about 40% w/w. In some embodiments, the percentage of glucose in the non-bee made honey composition is between about 25 and about 40% w/w. In some embodiments, the percentage of glucose in the non-bee made honey composition is between about 30 and about 40% w/w. In some embodiments, the percentage of glucose in the non-bee made honey composition is between about 30 and about 45% w/w. In some embodiments, the percentage of glucose in the plant-based honey composition is about 28%.

In some embodiments, the non-bee made honey comprises dextrose. In some percentage of dextrose in the final non-bee made honey is between about 20 and about 40% w/w. In some embodiments, the percentage of dextrose in the non-bee made honey is between about 25 and about 40% w/w. In some embodiments, the percentage of dextrose in the non-bee made honey composition is between about 30 and about 40% w/w. In some embodiments, the percentage of dextrose in the non-bee made honey is between about 30 and about 45% w/w. In some embodiments, the percentage of dextrose in the non-bee made honey composition is about 28%

In some embodiments, the total amount of fructose and glucose (sum of both) should not be less than 60 g/100 g.

In some embodiments, the honey base can include sucrose (0-5%), and/or maltose (0-10%).

### Acid Content

In some embodiments, the acids added to the base composition may include, but are not limited to: gluconic, succinic, citric, acetic, formic, p-coumaric, chlorogenic, gallic, syringic, caffeic, oxalic, lactic, ferulic, ellagic, vanillic, kojic, protocatechuic, malic, and cinnamic acid.

The pH of the final honey base solution may be within a range of 3.0 to 6.0. According to the Directive 2001/110/EC, Annex II, bee-made and therefore plant-based honeys, should not contain more than 50 milli-equivalents acid per 1000 g.

In some embodiments, the non-bee made honey composition may comprise gluconic acid, succinic acid, gallic acid, chlorogenic acid, cinnamic acid, kojic acid, ferulic acid, caffeic acid, citric acid, glucuno-delta-lactone, tartaric acid, lactic acid, hydroxybenzoic, protocatechuic, gentistic, sinapic, p-coumaric acid, or malic acid. In some embodiments, the plant based honey composition may comprise gluconic acid, succinic acid, gallic acid, chlorogenic acid, cinnamic acid, kojic acid, ferulic acid, caffeic acid, citric acid, glucuno-delta-lactone, tartaric acid, lactic acid, hydroxybenzoic, protocatechuic, gentistic, sinapic, p-coumaric acid, or malic acid.

In some embodiments, the non-bee made honey formulation comprises one or more organic acids comprising gluconic acid in an amount from about 0.001% to about 0.5% w/w. In some embodiments, the non-bee made honey formulation comprises one or more organic acids comprising gluconic acid in an amount from about 0.05% to about 0.5% w/w. In some embodiments, the gluconic acid is present in an amount from about 0.05% to about 1.00% w/w. In some embodiments, the gluconic acid is present in an amount from about 0.001% to about 1.00% w/w. In some embodiments, the gluconic acid is present in an amount from about 0.01% to about 1.00% w/w. In some embodiments, the gluconic acid is present in an amount from about 0.1% to about 1.00% w/w. In some embodiments, the gluconic acid is present in an amount from about 0.001% to about 0.1% w/w. In some embodiments, the non-bee made honey formulation comprises one or more organic acids comprising gluconic acid in an amount of about 0.1% w/w. In some embodiments, the gluconic acid is present in an amount of about 0.01% w/w. In some embodiments, the gluconic acid is present in an amount of about 0.001% w/w. In some embodiments, the non-bee made honey formulation comprises one or more organic acids comprising succinic acid in an amount from about 0.001% to about 0.5% w/w. In some embodiments, the succinic acid in an amount from about 0.001% to about 0.5% w/w. In some embodiments, the non-bee made honey formulation comprises one or more organic acids comprising succinic acid in an amount from about 0.05% to about 0.5% w/w. In some embodiments, the succinic acid is present in an amount from about 0.05% to about 0.5% w/w. In some embodiments, the succinic acid is present in an amount from about 0.005% to about 0.5% w/w. In some embodiments, the succinic acid is present in an amount from about 0.0005% to about 0.5% w/w. In some embodiments, the succinic acid is present in an amount from about 0.0005% to about 0.5% w/w. In some embodiments, the non-bee made honey formulation comprises one or more organic acids comprising succinic acid in an amount of about 0.010% w/w. In some embodiments, the non-bee made honey formulation comprises one or more organic acids comprising succinic acid in an amount of about 0.1% w/w. In some embodiments, the non-bee made honey formulation comprises one or more organic acids comprising succinic acid in an amount of about 0.001% w/w. In some embodiments, the non-bee made honey formulation comprises one or more organic acids comprising chlorogenic acid in an amount from about 0.001% to about 0.01% w/w. In some embodiments, the chlorogenic acid is present in an amount of about 0.001% to about 0.1% w/w. In some embodiments, the chlorogenic acid is present in an amount of about 0.01% to about 0.01% w/w. In some embodiments, the chlorogenic acid is present in an amount of about 0.00001% to about 0.010% w/w. In some embodiments, the chlorogenic acid is present in an amount of about 0.0001% to about 0.01% w/w. In some embodiments, the chlorogenic acid is present in an amount of about 0.0001% to about 0.1% w/w. In some embodiments, the non-bee made honey formulation comprises one or more organic acids comprising chlorogenic acid in an amount of about 0.004% w/w. In some embodiments, the non-bee made honey formulation comprises one or more organic acids comprising chlorogenic acid in an amount of about 0.01% w/w. In some embodiments, the non-bee made honey formulation comprises one or more organic acids comprising gallic acid in an amount from about 0.001% to about 0.01% w/w. In some embodiments, the gallic acid is present in an amount from about 0.001% to about 0.01% w/w. In some embodiments, the gallic acid is present in an amount from about 0.01% to about 0.1% w/w. In some embodiments, the gallic acid is present in an amount from about 0.001% to about 0.1% w/w. In some embodiments, the non-bee made honey formulation comprises one or more organic acids comprising gallic acid in an amount of about 0.005% w/w. In some embodiments, the gallic acid is present in an amount of about 0.01% w/w. In some embodiments, the gallic acid is present in an amount of about 0.001% w/w. In some embodiments, the non-bee made honey formulation comprises 50 milli-equivalents acid per 1000 g or less. In some embodiments, the non-bee made honey formulation comprises 500 milli-equivalents acid per 1000 g or less. In some embodiments, the non-bee made honey formulation comprises a pH from about 3.0 to about 6.0. In some embodiments, the non-bee made honey formulation comprises a pH from about 3.0 to about 6.0. In some embodiments, the non-bee made honey formulation comprises a pH from about 4.0 to about 6.0. In some embodiments, the non-bee made honey formulation comprises a pH from about 3.0 to about 5.0. In some embodiments, the non-bee made honey formulation comprises a pH from about 3.0 to about 4.0. In some embodiments, the non-bee made honey formulation comprises a pH from about 3.0 to about 5.5. In some embodiments, the non-bee made honey formulation comprises a pH from about 3.5 to about 6.0. In some embodiments, the pH of the formulation may be about 3 to about 3.25, about 3 to about 3.5, about 3 to about 3.75, about 3 to about 4, about 3 to about 4.25, about 3 to about 4.5, about 3 to about 4.75, about 3 to about 5, about 3 to about 5.25, about 3 to about 5.75, about 3 to about 6, about 3.25 to about 3.5, about 3.25 to about 3.75, about 3.25 to about 4, about 3.25 to about 4.25, about 3.25 to about 4.5, about 3.25 to about 4.75, about 3.25 to about 5, about 3.25 to about 5.25, about 3.25 to about 5.75, about 3.25 to about 6, about 3.5 to about 3.75, about 3.5 to about 4, about 3.5 to about 4.25, about 3.5 to about 4.5, about 3.5 to about 4.75, about 3.5 to about 5, about 3.5 to about 5.25, about 3.5 to about 5.75, about 3.5 to about 6, about 3.75 to about 4, about 3.75 to about 4.25, about 3.75 to about 4.5, about 3.75 to about 4.75, about 3.75 to about 5, about 3.75 to about 5.25, about 3.75 to about 5.75, about 3.75 to about 6, about 4 to about 4.25, about 4 to about 4.5, about 4 to about 4.75, about 4 to about 5, about 4 to about 5.25, about 4 to about 5.75, about 4 to about 6, about 4.25 to about 4.5, about 4.25 to about 4.75, about 4.25 to about 5, about 4.25 to about 5.25, about 4.25 to about 5.75, about 4.25 to about 6, about 4.5 to about 4.75, about 4.5 to about 5, about 4.5 to about 5.25, about 4.5 to about 5.75, about 4.5 to about 6, about 4.75 to about 5, about 4.75 to about 5.25, about 4.75 to about 5.75, about 4.75 to about 6, about 5 to about 5.25, about 5 to about 5.75, about 5 to about 6, about 5.25 to about 5.75, about 5.25 to about 6, or about 5.75 to about 6, including increments therein. In some embodiments, the non-bee made honey formulation comprises a pH of about 3.72.

In some embodiments, the pH of the formulation may be about 3 to about 6. In some embodiments, the pH of the formulation may be about 3 to about 3.25, about 3 to about 3.5, about 3 to about 3.75, about 3 to about 4, about 3 to about 4.25, about 3 to about 4.5, about 3 to about 4.75, about 3 to about 5, about 3 to about 5.25, about 3 to about 5.75, about 3 to about 6, about 3.25 to about 3.5, about 3.25 to about 3.75, about 3.25 to about 4, about 3.25 to about 4.25, about 3.25 to about 4.5, about 3.25 to about 4.75, about 3.25 to about 5, about 3.25 to about 5.25, about 3.25 to about 5.75, about 3.25 to about 6, about 3.5 to about 3.75, about 3.5 to about 4, about 3.5 to about 4.25, about 3.5 to about 4.5, about 3.5 to about 4.75, about 3.5 to about 5, about 3.5 to about 5.25, about 3.5 to about 5.75, about 3.5 to about 6, about 3.75 to about 4, about 3.75 to about 4.25, about 3.75 to about 4.5, about 3.75 to about 4.75, about 3.75 to about 5, about 3.75 to about 5.25, about 3.75 to about 5.75, about 3.75 to about 6, about 4 to about 4.25, about 4 to about 4.5, about 4 to about 4.75, about 4 to about 5, about 4 to about 5.25, about 4 to about 5.75, about 4 to about 6, about 4.25 to about 4.5, about 4.25 to about 4.75, about 4.25 to about 5, about 4.25 to about 5.25, about 4.25 to about 5.75, about 4.25 to about 6, about 4.5 to about 4.75, about 4.5 to about 5, about 4.5 to about 5.25, about 4.5 to about 5.75, about 4.5 to about 6, about 4.75 to about 5, about 4.75 to about 5.25, about 4.75 to about 5.75, about 4.75 to about 6, about 5 to about 5.25, about 5 to about 5.75, about 5 to about 6, about 5.25 to about 5.75, about 5.25 to about 6, or about 5.75 to about 6, including increments therein. In some embodiments, the plant-based honey has a pH of about 3 to about 3.25, about 3 to about 3.5, about 3 to about 3.75, about 3 to about 4, about 3 to about 4.25, about 3 to about 4.5, about 3 to about 4.75, about 3 to about 5, about 3 to about 5.25, about 3 to about 5.75, about 3 to about 6, about 3.25 to about 3.5, about 3.25 to about 3.75, about 3.25 to about 4, about 3.25 to about 4.25, about 3.25 to about 4.5, about 3.25 to about 4.75, about 3.25 to about 5, about 3.25 to about 5.25, about 3.25 to about 5.75, about 3.25 to about 6, about 3.5 to about 3.75, about 3.5 to about 4, about 3.5 to about 4.25, about 3.5 to about 4.5, about 3.5 to about 4.75, about 3.5 to about 5, about 3.5 to about 5.25, about 3.5 to about 5.75, about 3.5 to about 6, about 3.75 to about 4, about 3.75 to about 4.25, about 3.75 to about 4.5, about 3.75 to about 4.75, about 3.75 to about 5, about 3.75 to about 5.25, about 3.75 to about 5.75, about 3.75 to about 6, about 4 to about 4.25, about 4 to about 4.5, about 4 to about 4.75, about 4 to about 5, about 4 to about 5.25, about 4 to about 5.75, about 4 to about 6, about 4.25 to about 4.5, about 4.25 to about 4.75, about 4.25 to about 5, about 4.25 to about 5.25, about 4.25 to about 5.75, about 4.25 to about 6, about 4.5 to about 4.75, about 4.5 to about 5, about 4.5 to about 5.25, about 4.5 to about 5.75, about 4.5 to about 6, about 4.75 to about 5, about 4.75 to about 5.25, about 4.75 to about 5.75, about 4.75 to about 6, about 5 to about 5.25, about 5 to about 5.75, about 5 to about 6, about 5.25 to about 5.75, about 5.25 to about 6, or about 5.75 to about 6, including increments therein. In some embodiments, the pH of the formulation may be about 3, about 3.25, about 3.5, about 3.75, about 4, about 4.25, about 4.5, about 4.75, about 5, about 5.25, about 5.75, or about 6. In some embodiments, the pH of the formulation may be at least about 3, about 3.25, about 3.5, about 3.75, about 4, about 4.25, about 4.5, about 4.75, about 5, about 5.25, or about 5.75. In some embodiments, the pH of the formulation may be at most about 3.25, about 3.5, about 3.75, about 4, about 4.25, about 4.5, about 4.75, about 5, about 5.25, about 5.75, or about 6. In some embodiments, the non-bee made honey composition may have an acid content of between 5 and 0.001% w/w. In some embodiments, the non-bee made honey composition may have an acid content of between 2 and 0.1% w/w. In some embodiments, the non-bee made honey composition may have an acid content of between 1 and 0.1% w/w. In some embodiments, the non-bee made honey composition may have an acid content of between 1 and 0.01% w/w. In some embodiments, the non-bee made honey composition may have an acid content of between 0.5 and 0.05% w/w. In some embodiments, the non-bee made honey composition may have an acid content of between 0.1 and 0.01% w/w. In some embodiments, the non-bee made honey composition may have an acid content of between 0.01 and 0.001% w/w. In some embodiments, the plant-based honey composition may have an acid content of between 5 and 0.001% w/w. In some embodiments, the plant-based honey composition may have an acid content of between 2 and 0.1% w/w. In some embodiments, the plant-based honey composition may have an acid content of between 1 and 0.1% w/w. In some embodiments, the plant-based honey composition may have an acid content of between 1 and 0.01% w/w. In some embodiments, the plant-based honey composition may have an acid content of between 0.5 and 0.05% w/w. In some embodiments, the plant-based honey composition may have an acid content of between 0.1 and 0.01% w/w. In some embodiments, the plant-based honey composition may have an acid content of between 0.01 and 0.001% w/w.

In some embodiments, the pH of the plant based honey may be about 3 to about 6.

In some embodiments, the non-bee made honey composition may have an acid content of between 0.01 and 0.001% w/w. In some embodiments, the non-bee made honey composition has an acid concentration (e.g., H₃O⁺ concentration) of between 0.001 M and 0.0001 M. In some embodiments, the non-bee made honey composition has an acid concentration of between 0.001 M and 0.00001 M. In some embodiments, the non-bee made honey composition has an acid concentration of between 0.01 M and 0.0001 M. In some embodiments, the non-bee made honey composition has an acid concentration of between 0.0001 M and 0.00001 M. In some embodiments, the non-bee made honey composition has an acid concentration of between 0.001 M and 0.00001 M. In some embodiments, the non-bee made honey composition has an acid concentration of between 0.0001 M and 0.00001 M.

In some embodiments, the one or more acids are present in an amount of about 0.1% to about 4%. In some embodiments, the one or more acids are present in an amount of about 0.1% to about 0.4%, about 0.1% to about 0.6%, about 0.1% to about 0.8%, about 0.1% to about 1%, about 0.1% to about 1.4%, about 0.1% to about 1.8%, about 0.1% to about 2.4%, about 0.1% to about 2.8%, about 0.1% to about 3.2%, about 0.1% to about 3.6%, about 0.1% to about 4%, about 0.4% to about 0.6%, about 0.4% to about 0.8%, about 0.4% to about 1%, about 0.4% to about 1.4%, about 0.4% to about 1.8%, about 0.4% to about 2.4%, about 0.4% to about 2.8%, about 0.4% to about 3.2%, about 0.4% to about 3.6%, about 0.4% to about 4%, about 0.6% to about 0.8%, about 0.6% to about 1%, about 0.6% to about 1.4%, about 0.6% to about 1.8%, about 0.6% to about 2.4%, about 0.6% to about 2.8%, about 0.6% to about 3.2%, about 0.6% to about 3.6%, about 0.6% to about 4%, about 0.8% to about 1%, about 0.8% to about 1.4%, about 0.8% to about 1.8%, about 0.8% to about 2.4%, about 0.8% to about 2.8%, about 0.8% to about 3.2%, about 0.8% to about 3.6%, about 0.8% to about 4%, about 1% to about 1.4%, about 1% to about 1.8%, about 1% to about 2.4%, about 1% to about 2.8%, about 1% to about 3.2%, about 1% to about 3.6%, about 1% to about 4%, about 1.4% to about 1.8%, about 1.4% to about 2.4%, about 1.4% to about 2.8%, about 1.4% to about 3.2%, about 1.4% to about 3.6%, about 1.4% to about 4%, about 1.8% to about 2.4%, about 1.8% to about 2.8%, about 1.8% to about 3.2%, about 1.8% to about 3.6%, about 1.8% to about 4%, about 2.4% to about 2.8%, about 2.4% to about 3.2%, about 2.4% to about 3.6%, about 2.4% to about 4%, about 2.8% to about 3.2%, about 2.8% to about 3.6%, about 2.8% to about 4%, about 3.2% to about 3.6%, about 3.2% to about 4%, or about 3.6% to about 4%, including increments therein. In some embodiments, the one or more acids are present in an amount of about 0.1%, about 0.4%, about 0.6%, about 0.8%, about 1%, about 1.4%, about 1.8%, about 2.4%, about 2.8%, about 3.2%, about 3.6%, or about 4%. In some embodiments, the one or more acids are present in an amount of at least about 0.1%, about 0.4%, about 0.6%, about 0.8% about 1%, about 1.4%, about 1.8%, about 2.4%, about 2.8%, about 3.2%, or about 3.6%. In some embodiments, the one or more acids are present in an amount of at most about 0.4%, about 0.6%, about 0.8%, about 1%, about 1.4%, about 1.8%, about 2.4%, about 2.8%, about 3.2%, about 3.6%, or about 4%.

In some embodiments, the non-bee made honey composition comprises organic acids in an amount between about 0.0001% w/w to about 5% w/w. In some embodiments, the non-bee made honey composition comprises organic acids in an amount between about 0.0001% w/w to about 0.0005% w/w, about 0.0001% w/w to about 0.001% w/w, about 0.0001% w/w to about 0.005% w/w, about 0.0001% w/w to about 0.01% w/w, about 0.0001% w/w to about 0.05% w/w, about 0.0001% w/w to about 0.1% w/w, about 0.0001% w/w to about 1% w/w, about 0.0001% w/w to about 2% w/w, about 0.0001% w/w to about 3% w/w, about 0.0001% w/w to about 4% w/w, about 0.0001% w/w to about 5% w/w, about 0.0005% w/w to about 0.001% w/w, about 0.0005% w/w to about 0.005% w/w, about 0.0005% w/w to about 0.01% w/w, about 0.0005% w/w to about 0.05% w/w, about 0.0005% w/w to about 0.1% w/w, about 0.0005% w/w to about 1% w/w, about 0.0005% w/w to about 2% w/w, about 0.0005% w/w to about 3% w/w, about 0.0005% w/w to about 4% w/w, about 0.0005% w/w to about 5% w/w, about 0.001% w/w to about 0.005% w/w, about 0.001% w/w to about 0.01% w/w, about 0.001% w/w to about 0.05% w/w, about 0.001% w/w to about 0.1% w/w, about 0.001% w/w to about 1% w/w, about 0.001% w/w to about 2% w/w, about 0.001% w/w to about 3% w/w, about 0.001% w/w to about 4% w/w, about 0.001% w/w to about 5% w/w, about 0.005% w/w to about 0.01% w/w, about 0.005% w/w to about 0.05% w/w, about 0.005% w/w to about 0.1% w/w, about 0.005% w/w to about 1% w/w, about 0.005% w/w to about 2% w/w, about 0.005% w/w to about 3% w/w, about 0.005% w/w to about 4% w/w, about 0.005% w/w to about 5% w/w, about 0.01% w/w to about 0.05% w/w, about 0.01% w/w to about 0.1% w/w, about 0.01% w/w to about 1% w/w, about 0.01% w/w to about 2% w/w, about 0.01% w/w to about 3% w/w, about 0.01% w/w to about 4% w/w, about 0.01% w/w to about 5% w/w, about 0.05% w/w to about 0.1% w/w, about 0.05% w/w to about 1% w/w, about 0.05% w/w to about 2% w/w, about 0.05% w/w to about 3% w/w, about 0.05% w/w to about 4% w/w, about 0.05% w/w to about 5% w/w, about 0.1% w/w to about 1% w/w, about 0.1% w/w to about 2% w/w, about 0.1% w/w to about 3% w/w, about 0.1% w/w to about 4% w/w, about 0.1% w/w to about 5% w/w, about 1% w/w to about 2% w/w, about 1% w/w to about 3% w/w, about 1% w/w to about 4% w/w, about 1% w/w to about 5% w/w, about 2% w/w to about 3% w/w, about 2% w/w to about 4% w/w, about 2% w/w to about 5% w/w, about 3% w/w to about 4% w/w, about 3% w/w to about 5% w/w, or about 4% w/w to about 5% w/w, including increments therein. In some embodiments, the non-bee made honey composition comprises organic acids in an amount between about 0.0001% w/w, about 0.0005% w/w, about 0.001% w/w, about 0.005% w/w, about 0.01% w/w, about 0.05% w/w, about 0.1% w/w, about 1% w/w, about 2% w/w, about 3% w/w, about 4% w/w, or about 5% w/w. In some embodiments, the non-bee made honey composition comprises organic acids in an amount between at least about 0.0001% w/w, about 0.0005% w/w, about 0.001% w/w, about 0.005% w/w, about 0.01% w/w, about 0.05% w/w, about 0.1% w/w, about 1% w/w, about 2% w/w, about 3% w/w, or about 4% w/w. In some embodiments, the non-bee made honey composition comprises organic acids in an amount between at most about 0.0005% w/w, about 0.001% w/w, about 0.005% w/w, about 0.01% w/w, about 0.05% w/w, about 0.1% w/w, about 1% w/w, about 2% w/w, about 3% w/w, about 4% w/w, or about 5% w/w. In some embodiments, the one or more acids are present in an amount of at least about 0.1%, at least about 0.4%, at least about 0.6%, at least about 0.8%, at least about 1%, at least about 1.4%, at least about 1.8%, at least about 2.4%, at least about 2.8%, at least about 3.2%, or at least about 3.6%. In some embodiments, the one or more acids are present in an amount of at most about 0.4%, at most about 0.6%, at most about 0.8%, at most about 1%, at most about 1.4%, at most about 1.8%, at most about 2.4%, at most about 2.8%, at most about 3.2%, at most about 3.6%, or at most about 4%.

In some embodiments, the plant-based honey composition comprises organic acids in an amount between about 0.0001% w/w to about 5% w/w. In some embodiments, the plant-based honey composition comprises organic acids in an amount between about 0.0001% w/w to about 0.0005% w/w, about 0.0001% w/w to about 0.001% w/w, about 0.0001% w/w to about 0.005% w/w, about 0.0001% w/w to about 0.01% w/w, about 0.0001% w/w to about 0.05% w/w, about 0.0001% w/w to about 0.1% w/w, about 0.0001% w/w to about 1% w/w, about 0.0001% w/w to about 2% w/w, about 0.0001% w/w to about 3% w/w, about 0.0001% w/w to about 4% w/w, about 0.0001% w/w to about 5% w/w, about 0.0005% w/w to about 0.001% w/w, about 0.0005% w/w to about 0.005% w/w, about 0.0005% w/w to about 0.01% w/w, about 0.0005% w/w to about 0.05% w/w, about 0.0005% w/w to about 0.1% w/w, about 0.0005% w/w to about 1% w/w, about 0.0005% w/w to about 2% w/w, about 0.0005% w/w to about 3% w/w, about 0.0005% w/w to about 4% w/w, about 0.0005% w/w to about 5% w/w, about 0.001% w/w to about 0.005% w/w, about 0.001% w/w to about 0.01% w/w, about 0.001% w/w to about 0.05% w/w, about 0.001% w/w to about 0.1% w/w, about 0.001% w/w to about 1% w/w, about 0.001% w/w to about 2% w/w, about 0.001% w/w to about 3% w/w, about 0.001% w/w to about 4% w/w, about 0.001% w/w to about 5% w/w, about 0.005% w/w to about 0.01% w/w, about 0.005% w/w to about 0.05% w/w, about 0.005% w/w to about 0.1% w/w, about 0.005% w/w to about 1% w/w, about 0.005% w/w to about 2% w/w, about 0.005% w/w to about 3% w/w, about 0.005% w/w to about 4% w/w, about 0.005% w/w to about 5% w/w, about 0.01% w/w to about 0.05% w/w, about 0.01% w/w to about 0.1% w/w, about 0.01% w/w to about 1% w/w, about 0.01% w/w to about 2% w/w, about 0.01% w/w to about 3% w/w, about 0.01% w/w to about 4% w/w, about 0.01% w/w to about 5% w/w, about 0.05% w/w to about 0.1% w/w, about 0.05% w/w to about 1% w/w, about 0.05% w/w to about 2% w/w, about 0.05% w/w to about 3% w/w, about 0.05% w/w to about 4% w/w, about 0.05% w/w to about 5% w/w, about 0.1% w/w to about 1% w/w, about 0.1% w/w to about 2% w/w, about 0.1% w/w to about 3% w/w, about 0.1% w/w to about 4% w/w, about 0.1% w/w to about 5% w/w, about 1% w/w to about 2% w/w, about 1% w/w to about 3% w/w, about 1% w/w to about 4% w/w, about 1% w/w to about 5% w/w, about 2% w/w to about 3% w/w, about 2% w/w to about 4% w/w, about 2% w/w to about 5% w/w, about 3% w/w to about 4% w/w, about 3% w/w to about 5% w/w, or about 4% w/w to about 5% w/w, including increments therein. In some embodiments, the plant-based honey composition comprises organic acids in an amount between about 0.0001% w/w, about 0.0005% w/w, about 0.001% w/w, about 0.005% w/w, about 0.01% w/w, about 0.05% w/w, about 0.1% w/w, about 1% w/w, about 2% w/w, about 3% w/w, about 4% w/w, or about 5% w/w. In some embodiments, the plant-based honey composition comprises organic acids in an amount between at least about 0.0001% w/w, about 0.0005% w/w, about 0.001% w/w, about 0.005% w/w, about 0.01% w/w, about 0.05% w/w, about 0.1% w/w, about 1% w/w, about 2% w/w, about 3% w/w, or about 4% w/w. In some embodiments, the plant-based honey composition comprises organic acids in an amount between at most about 0.0005% w/w, about 0.001% w/w, about 0.005% w/w, about 0.01% w/w, about 0.05% w/w, about 0.1% w/w, about 1% w/w, about 2% w/w, about 3% w/w, about 4% w/w, or about 5% w/w. In some embodiments, the one or more acids are present in an amount of at least about 0.1%, at least about 0.4%, at least about 0.6%, at least about 0.8%, at least about 1%, at least about 1.4%, at least about 1.8%, at least about 2.4%, at least about 2.8%, at least about 3.2%, or at least about 3.6%. In some embodiments, the one or more acids are present in an amount of at most about 0.4%, at most about 0.6%, at most about 0.8%, at most about 1%, at most about 1.4%, at most about 1.8%, at most about 2.4%, at most about 2.8%, at most about 3.2%, at most about 3.6%, or at most about 4%.

In some embodiments, the non-bee made honey composition comprises gallic acid in an amount between about 0.001% w/w to about 1% w/w. In some embodiments, the non-bee made honey composition comprises gallic acid in an amount between about 0.001% w/w to about 0.003% w/w, about 0.001% w/w to about 0.005% w/w, about 0.001% w/w to about 0.007% w/w, about 0.001% w/w to about 0.01% w/w, about 0.001% w/w to about 0.05% w/w, about 0.001% w/w to about 0.1% w/w, about 0.001% w/w to about 0.3% w/w, about 0.001% w/w to about 0.5% w/w, about 0.001% w/w to about 0.7% w/w, about 0.001% w/w to about 0.9% w/w, about 0.001% w/w to about 1% w/w, about 0.003% w/w to about 0.005% w/w, about 0.003% w/w to about 0.007% w/w, about 0.003% w/w to about 0.01% w/w, about 0.003% w/w to about 0.05% w/w, about 0.003% w/w to about 0.1% w/w, about 0.003% w/w to about 0.3% w/w, about 0.003% w/w to about 0.5% w/w, about 0.003% w/w to about 0.7% w/w, about 0.003% w/w to about 0.9% w/w, about 0.003% w/w to about 1% w/w, about 0.005% w/w to about 0.007% w/w, about 0.005% w/w to about 0.01% w/w, about 0.005% w/w to about 0.05% w/w, about 0.005% w/w to about 0.1% w/w, about 0.005% w/w to about 0.3% w/w, about 0.005% w/w to about 0.5% w/w, about 0.005% w/w to about 0.7% w/w, about 0.005% w/w to about 0.9% w/w, about 0.005% w/w to about 1% w/w, about 0.007% w/w to about 0.01% w/w, about 0.007% w/w to about 0.05% w/w, about 0.007% w/w to about 0.1% w/w, about 0.007% w/w to about 0.3% w/w, about 0.007% w/w to about 0.5% w/w, about 0.007% w/w to about 0.7% w/w, about 0.007% w/w to about 0.9% w/w, about 0.007% w/w to about 1% w/w, about 0.01% w/w to about 0.05% w/w, about 0.01% w/w to about 0.1% w/w, about 0.01% w/w to about 0.3% w/w, about 0.01% w/w to about 0.5% w/w, about 0.01% w/w to about 0.7% w/w, about 0.01% w/w to about 0.9% w/w, about 0.01% w/w to about 1% w/w, about 0.05% w/w to about 0.1% w/w, about 0.05% w/w to about 0.3% w/w, about 0.05% w/w to about 0.5% w/w, about 0.05% w/w to about 0.7% w/w, about 0.05% w/w to about 0.9% w/w, about 0.05% w/w to about 1% w/w, about 0.1% w/w to about 0.3% w/w, about 0.1% w/w to about 0.5% w/w, about 0.1% w/w to about 0.7% w/w, about 0.1% w/w to about 0.9% w/w, about 0.1% w/w to about 1% w/w, about 0.3% w/w to about 0.5% w/w, about 0.3% w/w to about 0.7% w/w, about 0.3% w/w to about 0.9% w/w, about 0.3% w/w to about 1% w/w, about 0.5% w/w to about 0.7% w/w, about 0.5% w/w to about 0.9% w/w, about 0.5% w/w to about 1% w/w, about 0.7% w/w to about 0.9% w/w, about 0.7% w/w to about 1% w/w, or about 0.9% w/w to about 1% w/w, including increments therein. In some embodiments, the non-bee made honey composition comprises gallic acid in an amount between about 0.001% w/w, about 0.003% w/w, about 0.005% w/w, about 0.007% w/w, about 0.01% w/w, about 0.05% w/w, about 0.1% w/w, about 0.3% w/w, about 0.5% w/w, about 0.7% w/w, about 0.9% w/w, or about 1% w/w. In some embodiments, the non-bee made honey composition comprises gallic acid in an amount between at least about 0.001% w/w, about 0.003% w/w, about 0.005% w/w, about 0.007% w/w, about 0.01% w/w, about 0.05% w/w, about 0.1% w/w, about 0.3% w/w, about 0.5% w/w, about 0.7% w/w, or about 0.9% w/w. In some embodiments, the non-bee made honey composition comprises gallic acid in an amount between at most about 0.003% w/w, about 0.005% w/w, about 0.007% w/w, about 0.01% w/w, about 0.05% w/w, about 0.1% w/w, about 0.3% w/w, about 0.5% w/w, about 0.7% w/w, about 0.9% w/w, or about 1% w/w. In some embodiments, the non-bee made honey composition comprises between 0.01 and 0.001% w/w gallic acid. In some embodiments, the non-bee made honey composition comprises between 0.001 and 0.0001% w/w gallic acid. In some embodiments, the plant-based honey composition comprises gallic acid in an amount between about 0.001% w/w to about 1% w/w. In some embodiments, the plant-based honey composition comprises gallic acid in an amount between about 0.001% w/w to about 0.003% w/w, about 0.001% w/w to about 0.005% w/w, about 0.001% w/w to about 0.007% w/w, about 0.001% w/w to about 0.01% w/w, about 0.001% w/w to about 0.05% w/w, about 0.001% w/w to about 0.1% w/w, about 0.001% w/w to about 0.3% w/w, about 0.001% w/w to about 0.5% w/w, about 0.001% w/w to about 0.7% w/w, about 0.001% w/w to about 0.9% w/w, about 0.001% w/w to about 1% w/w, about 0.003% w/w to about 0.005% w/w, about 0.003% w/w to about 0.007% w/w, about 0.003% w/w to about 0.01% w/w, about 0.003% w/w to about 0.05% w/w, about 0.003% w/w to about 0.1% w/w, about 0.003% w/w to about 0.3% w/w, about 0.003% w/w to about 0.5% w/w, about 0.003% w/w to about 0.7% w/w, about 0.003% w/w to about 0.9% w/w, about 0.003% w/w to about 1% w/w, about 0.005% w/w to about 0.007% w/w, about 0.005% w/w to about 0.01% w/w, about 0.005% w/w to about 0.05% w/w, about 0.005% w/w to about 0.1% w/w, about 0.005% w/w to about 0.3% w/w, about 0.005% w/w to about 0.5% w/w, about 0.005% w/w to about 0.7% w/w, about 0.005% w/w to about 0.9% w/w, about 0.005% w/w to about 1% w/w, about 0.007% w/w to about 0.01% w/w, about 0.007% w/w to about 0.05% w/w, about 0.007% w/w to about 0.1% w/w, about 0.007% w/w to about 0.3% w/w, about 0.007% w/w to about 0.5% w/w, about 0.007% w/w to about 0.7% w/w, about 0.007% w/w to about 0.9% w/w, about 0.007% w/w to about 1% w/w, about 0.01% w/w to about 0.05% w/w, about 0.01% w/w to about 0.1% w/w, about 0.01% w/w to about 0.3% w/w, about 0.01% w/w to about 0.5% w/w, about 0.01% w/w to about 0.7% w/w, about 0.01% w/w to about 0.9% w/w, about 0.01% w/w to about 1% w/w, about 0.05% w/w to about 0.1% w/w, about 0.05% w/w to about 0.3% w/w, about 0.05% w/w to about 0.5% w/w, about 0.05% w/w to about 0.7% w/w, about 0.05% w/w to about 0.9% w/w, about 0.05% w/w to about 1% w/w, about 0.1% w/w to about 0.3% w/w, about 0.1% w/w to about 0.5% w/w, about 0.1% w/w to about 0.7% w/w, about 0.1% w/w to about 0.9% w/w, about 0.1% w/w to about 1% w/w, about 0.3% w/w to about 0.5% w/w, about 0.3% w/w to about 0.7% w/w, about 0.3% w/w to about 0.9% w/w, about 0.3% w/w to about 1% w/w, about 0.5% w/w to about 0.7% w/w, about 0.5% w/w to about 0.9% w/w, about 0.5% w/w to about 1% w/w, about 0.7% w/w to about 0.9% w/w, about 0.7% w/w to about 1% w/w, or about 0.9% w/w to about 1% w/w, including increments therein. In some embodiments, the plant-based honey composition comprises gallic acid in an amount between about 0.001% w/w, about 0.003% w/w, about 0.005% w/w, about 0.007% w/w, about 0.01% w/w, about 0.05% w/w, about 0.1% w/w, about 0.3% w/w, about 0.5% w/w, about 0.7% w/w, about 0.9% w/w, or about 1% w/w. In some embodiments, the plant-based honey composition comprises gallic acid in an amount between at least about 0.001% w/w, about 0.003% w/w, about 0.005% w/w, about 0.007% w/w, about 0.01% w/w, about 0.05% w/w, about 0.1% w/w, about 0.3% w/w, about 0.5% w/w, about 0.7% w/w, or about 0.9% w/w. In some embodiments, the plant-based honey composition comprises gallic acid in an amount between at most about 0.003% w/w, about 0.005% w/w, about 0.007% w/w, about 0.01% w/w, about 0.05% w/w, about 0.1% w/w, about 0.3% w/w, about 0.5% w/w, about 0.7% w/w, about 0.9% w/w, or about 1% w/w. In some embodiments, the plant-based honey composition comprises between 0.01 and 0.001% w/w gallic acid. In some embodiments, the plant-based honey composition comprises between 0.001 and 0.0001% w/w gallic acid.

In some embodiments, the non-bee made honey composition comprises chlorogenic acid in an amount between about 0.001% w/w to about 1% w/w. In some embodiments, the non-bee made honey composition comprises chlorogenic acid in an amount between about 0.001% w/w to about 0.003% w/w, about 0.001% w/w to about 0.0042% w/w, about 0.001% w/w to about 0.005% w/w, about 0.001% w/w to about 0.0075% w/w, about 0.001% w/w to about 0.01% w/w, about 0.001% w/w to about 0.05% w/w, about 0.001% w/w to about 0.1% w/w, about 0.001% w/w to about 0.5% w/w, about 0.001% w/w to about 0.7% w/w, about 0.001% w/w to about 0.9% w/w, about 0.001% w/w to about 1% w/w, about 0.003% w/w to about 0.0042% w/w, about 0.003% w/w to about 0.005% w/w, about 0.003% w/w to about 0.0075% w/w, about 0.003% w/w to about 0.01% w/w, about 0.003% w/w to about 0.05% w/w, about 0.003% w/w to about 0.1% w/w, about 0.003% w/w to about 0.5% w/w, about 0.003% w/w to about 0.7% w/w, about 0.003% w/w to about 0.9% w/w, about 0.003% w/w to about 1% w/w, about 0.0042% w/w to about 0.005% w/w, about 0.0042% w/w to about 0.0075% w/w, about 0.0042% w/w to about 0.01% w/w, about 0.0042% w/w to about 0.05% w/w, about 0.0042% w/w to about 0.1% w/w, about 0.0042% w/w to about 0.5% w/w, about 0.0042% w/w to about 0.7% w/w, about 0.0042% w/w to about 0.9% w/w, about 0.0042% w/w to about 1% w/w, about 0.005% w/w to about 0.0075% w/w, about 0.005% w/w to about 0.01% w/w, about 0.005% w/w to about 0.05% w/w, about 0.005% w/w to about 0.1% w/w, about 0.005% w/w to about 0.5% w/w, about 0.005% w/w to about 0.7% w/w, about 0.005% w/w to about 0.9% w/w, about 0.005% w/w to about 1% w/w, about 0.0075% w/w to about 0.01% w/w, about 0.0075% w/w to about 0.05% w/w, about 0.0075% w/w to about 0.1% w/w, about 0.0075% w/w to about 0.5% w/w, about 0.0075% w/w to about 0.7% w/w, about 0.0075% w/w to about 0.9% w/w, about 0.0075% w/w to about 1% w/w, about 0.01% w/w to about 0.05% w/w, about 0.01% w/w to about 0.1% w/w, about 0.01% w/w to about 0.5% w/w, about 0.01% w/w to about 0.7% w/w, about 0.01% w/w to about 0.9% w/w, about 0.01% w/w to about 1% w/w, about 0.05% w/w to about 0.1% w/w, about 0.05% w/w to about 0.5% w/w, about 0.05% w/w to about 0.7% w/w, about 0.05% w/w to about 0.9% w/w, about 0.05% w/w to about 1% w/w, about 0.1% w/w to about 0.5% w/w, about 0.1% w/w to about 0.7% w/w, about 0.1% w/w to about 0.9% w/w, about 0.1% w/w to about 1% w/w, about 0.5% w/w to about 0.7% w/w, about 0.5% w/w to about 0.9% w/w, about 0.5% w/w to about 1% w/w, about 0.7% w/w to about 0.9% w/w, about 0.7% w/w to about 1% w/w, or about 0.9% w/w to about 1% w/w, including increments therein. In some embodiments, the non-bee made honey composition comprises chlorogenic acid in an amount between about 0.001% w/w, about 0.003% w/w, about 0.0042% w/w, about 0.005% w/w, about 0.0075% w/w, about 0.01% w/w, about 0.05% w/w, about 0.1% w/w, about 0.5% w/w, about 0.7% w/w, about 0.9% w/w, or about 1% w/w. In some embodiments, the non-bee made honey composition comprises chlorogenic acid in an amount between at least about 0.001% w/w, about 0.003% w/w, about 0.0042% w/w, about 0.005% w/w, about 0.0075% w/w, about 0.01% w/w, about 0.05% w/w, about 0.1% w/w, about 0.5% w/w, about 0.7% w/w, or about 0.9% w/w. In some embodiments, the non-bee made honey composition comprises chlorogenic acid in an amount between at most about 0.003% w/w, about 0.0042% w/w, about 0.005% w/w, about 0.0075% w/w, about 0.01% w/w, about 0.05% w/w, about 0.1% w/w, about 0.5% w/w, about 0.7% w/w, about 0.9% w/w, or about 1% w/w. In some embodiments, the non-bee made honey composition comprises between 0.01 and 0.0001% w/w chlorogenic acid. In some embodiments, the non-bee made honey composition comprises between 0.01 and 0.001% w/w chlorogenic acid. In some embodiments, the non-bee made honey composition comprises between 0.001 and 0.0001% w/w chlorogenic acid. In some embodiments, the plant-based honey composition comprises chlorogenic acid in an amount between about 0.001% w/w to about 1% w/w. In some embodiments, the plant-based honey composition comprises chlorogenic acid in an amount between about 0.001% w/w to about 0.003% w/w, about 0.001% w/w to about 0.0042% w/w, about 0.001% w/w to about 0.005% w/w, about 0.001% w/w to about 0.0075% w/w, about 0.001% w/w to about 0.01% w/w, about 0.001% w/w to about 0.05% w/w, about 0.001% w/w to about 0.1% w/w, about 0.001% w/w to about 0.5% w/w, about 0.001% w/w to about 0.7% w/w, about 0.001% w/w to about 0.9% w/w, about 0.001% w/w to about 1% w/w, about 0.003% w/w to about 0.0042% w/w, about 0.003% w/w to about 0.005% w/w, about 0.003% w/w to about 0.0075% w/w, about 0.003% w/w to about 0.01% w/w, about 0.003% w/w to about 0.05% w/w, about 0.003% w/w to about 0.1% w/w, about 0.003% w/w to about 0.5% w/w, about 0.003% w/w to about 0.7% w/w, about 0.003% w/w to about 0.9% w/w, about 0.003% w/w to about 1% w/w, about 0.0042% w/w to about 0.005% w/w, about 0.0042% w/w to about 0.0075% w/w, about 0.0042% w/w to about 0.01% w/w, about 0.0042% w/w to about 0.05% w/w, about 0.0042% w/w to about 0.1% w/w, about 0.0042% w/w to about 0.5% w/w, about 0.0042% w/w to about 0.7% w/w, about 0.0042% w/w to about 0.9% w/w, about 0.0042% w/w to about 1% w/w, about 0.005% w/w to about 0.0075% w/w, about 0.005% w/w to about 0.01% w/w, about 0.005% w/w to about 0.05% w/w, about 0.005% w/w to about 0.1% w/w, about 0.005% w/w to about 0.5% w/w, about 0.005% w/w to about 0.7% w/w, about 0.005% w/w to about 0.9% w/w, about 0.005% w/w to about 1% w/w, about 0.0075% w/w to about 0.01% w/w, about 0.0075% w/w to about 0.05% w/w, about 0.0075% w/w to about 0.1% w/w, about 0.0075% w/w to about 0.5% w/w, about 0.0075% w/w to about 0.7% w/w, about 0.0075% w/w to about 0.9% w/w, about 0.0075% w/w to about 1% w/w, about 0.01% w/w to about 0.05% w/w, about 0.01% w/w to about 0.1% w/w, about 0.01% w/w to about 0.5% w/w, about 0.01% w/w to about 0.7% w/w, about 0.01% w/w to about 0.9% w/w, about 0.01% w/w to about 1% w/w, about 0.05% w/w to about 0.1% w/w, about 0.05% w/w to about 0.5% w/w, about 0.05% w/w to about 0.7% w/w, about 0.05% w/w to about 0.9% w/w, about 0.05% w/w to about 1% w/w, about 0.1% w/w to about 0.5% w/w, about 0.1% w/w to about 0.7% w/w, about 0.1% w/w to about 0.9% w/w, about 0.1% w/w to about 1% w/w, about 0.5% w/w to about 0.7% w/w, about 0.5% w/w to about 0.9% w/w, about 0.5% w/w to about 1% w/w, about 0.7% w/w to about 0.9% w/w, about 0.7% w/w to about 1% w/w, or about 0.9% w/w to about 1% w/w, including increments therein. In some embodiments, the plant-based honey composition comprises chlorogenic acid in an amount between about 0.001% w/w, about 0.003% w/w, about 0.0042% w/w, about 0.005% w/w, about 0.0075% w/w, about 0.01% w/w, about 0.05% w/w, about 0.1% w/w, about 0.5% w/w, about 0.7% w/w, about 0.9% w/w, or about 1% w/w. In some embodiments, the plant-based honey composition comprises chlorogenic acid in an amount between at least about 0.001% w/w, about 0.003% w/w, about 0.0042% w/w, about 0.005% w/w, about 0.0075% w/w, about 0.01% w/w, about 0.05% w/w, about 0.1% w/w, about 0.5% w/w, about 0.7% w/w, or about 0.9% w/w. In some embodiments, the plant-based honey composition comprises chlorogenic acid in an amount between at most about 0.003% w/w, about 0.0042% w/w, about 0.005% w/w, about 0.0075% w/w, about 0.01% w/w, about 0.05% w/w, about 0.1% w/w, about 0.5% w/w, about 0.7% w/w, about 0.9% w/w, or about 1% w/w. In some embodiments, the plant-based honey composition comprises between 0.01 and 0.0001% w/w chlorogenic acid. In some embodiments, the plant-based honey composition comprises between 0.01 and 0.001% w/w chlorogenic acid. In some embodiments, the plant-based honey composition comprises between 0.001 and 0.0001% w/w chlorogenic acid.

In some embodiments, the non-bee made honey composition comprises succinic acid in an amount between about 0.001% w/w to about 1% w/w. In some embodiments, the non-bee made honey composition comprises succinic acid in an amount between about 0.001% w/w to about 0.005% w/w, about 0.001% w/w to about 0.01% w/w, about 0.001% w/w to about 0.0108% w/w, about 0.001% w/w to about 0.015% w/w, about 0.001% w/w to about 0.02% w/w, about 0.001% w/w to about 0.05% w/w, about 0.001% w/w to about 0.08% w/w, about 0.001% w/w to about 0.1% w/w, about 0.001% w/w to about 0.4% w/w, about 0.001% w/w to about 0.8% w/w, about 0.001% w/w to about 1% w/w, about 0.005% w/w to about 0.01% w/w, about 0.005% w/w to about 0.0108% w/w, about 0.005% w/w to about 0.015% w/w, about 0.005% w/w to about 0.02% w/w, about 0.005% w/w to about 0.05% w/w, about 0.005% w/w to about 0.08% w/w, about 0.005% w/w to about 0.1% w/w, about 0.005% w/w to about 0.4% w/w, about 0.005% w/w to about 0.8% w/w, about 0.005% w/w to about 1% w/w, about 0.01% w/w to about 0.0108% w/w, about 0.01% w/w to about 0.015% w/w, about 0.01% w/w to about 0.02% w/w, about 0.01% w/w to about 0.05% w/w, about 0.01% w/w to about 0.08% w/w, about 0.01% w/w to about 0.1% w/w, about 0.01% w/w to about 0.4% w/w, about 0.01% w/w to about 0.8% w/w, about 0.01% w/w to about 1% w/w, about 0.0108% w/w to about 0.015% w/w, about 0.0108% w/w to about 0.02% w/w, about 0.0108% w/w to about 0.05% w/w, about 0.0108% w/w to about 0.08% w/w, about 0.0108% w/w to about 0.1% w/w, about 0.0108% w/w to about 0.4% w/w, about 0.0108% w/w to about 0.8% w/w, about 0.0108% w/w to about 1% w/w, about 0.015% w/w to about 0.02% w/w, about 0.015% w/w to about 0.05% w/w, about 0.015% w/w to about 0.08% w/w, about 0.015% w/w to about 0.1% w/w, about 0.015% w/w to about 0.4% w/w, about 0.015% w/w to about 0.8% w/w, about 0.015% w/w to about 1% w/w, about 0.02% w/w to about 0.05% w/w, about 0.02% w/w to about 0.08% w/w, about 0.02% w/w to about 0.1% w/w, about 0.02% w/w to about 0.4% w/w, about 0.02% w/w to about 0.8% w/w, about 0.02% w/w to about 1% w/w, about 0.05% w/w to about 0.08% w/w, about 0.05% w/w to about 0.1% w/w, about 0.05% w/w to about 0.4% w/w, about 0.05% w/w to about 0.8% w/w, about 0.05% w/w to about 1% w/w, about 0.08% w/w to about 0.1% w/w, about 0.08% w/w to about 0.4% w/w, about 0.08% w/w to about 0.8% w/w, about 0.08% w/w to about 1% w/w, about 0.1% w/w to about 0.4% w/w, about 0.1% w/w to about 0.8% w/w, about 0.1% w/w to about 1% w/w, about 0.4% w/w to about 0.8% w/w, about 0.4% w/w to about 1% w/w, or about 0.8% w/w to about 1% w/w, including increments therein. In some embodiments, the non-bee made honey composition comprises succinic acid in an amount between about 0.001% w/w, about 0.005% w/w, about 0.01% w/w, about 0.0108% w/w, about 0.015% w/w, about 0.02% w/w, about 0.05% w/w, about 0.08% w/w, about 0.1% w/w, about 0.4% w/w, about 0.8% w/w, or about 1% w/w. In some embodiments, the non-bee made honey composition comprises succinic acid in an amount between at least about 0.001% w/w, about 0.005% w/w, about 0.01% w/w, about 0.0108% w/w, about 0.015% w/w, about 0.02% w/w, about 0.05% w/w, about 0.08% w/w, about 0.1% w/w, about 0.4% w/w, or about 0.8% w/w. In some embodiments, the non-bee made honey composition comprises succinic acid in an amount between at most about 0.005% w/w, about 0.01% w/w, about 0.0108% w/w, about 0.015% w/w, about 0.02% w/w, about 0.05% w/w, about 0.08% w/w, about 0.1% w/w, about 0.4% w/w, about 0.8% w/w, or about 1% w/w. In some embodiments, the non-bee made honey composition comprises between 0.01 and 0.0001% w/w succinic acid. In some embodiments, the non-bee made honey composition comprises between 0.01 and 0.001% w/w succinic acid. In some embodiments, the non-bee made honey composition comprises between 0.001 and 0.0001% w/w succinic acid. In some embodiments, the plant-based honey composition comprises succinic acid in an amount between about 0.001% w/w to about 1% w/w. In some embodiments, the plant-based honey composition comprises succinic acid in an amount between about 0.001% w/w to about 0.005% w/w, about 0.001% w/w to about 0.01% w/w, about 0.001% w/w to about 0.0108% w/w, about 0.001% w/w to about 0.015% w/w, about 0.001% w/w to about 0.02% w/w, about 0.001% w/w to about 0.05% w/w, about 0.001% w/w to about 0.08% w/w, about 0.001% w/w to about 0.1% w/w, about 0.001% w/w to about 0.4% w/w, about 0.001% w/w to about 0.8% w/w, about 0.001% w/w to about 1% w/w, about 0.005% w/w to about 0.01% w/w, about 0.005% w/w to about 0.0108% w/w, about 0.005% w/w to about 0.015% w/w, about 0.005% w/w to about 0.02% w/w, about 0.005% w/w to about 0.05% w/w, about 0.005% w/w to about 0.08% w/w, about 0.005% w/w to about 0.1% w/w, about 0.005% w/w to about 0.4% w/w, about 0.005% w/w to about 0.8% w/w, about 0.005% w/w to about 1% w/w, about 0.01% w/w to about 0.0108% w/w, about 0.01% w/w to about 0.015% w/w, about 0.01% w/w to about 0.02% w/w, about 0.01% w/w to about 0.05% w/w, about 0.01% w/w to about 0.08% w/w, about 0.01% w/w to about 0.1% w/w, about 0.01% w/w to about 0.4% w/w, about 0.01% w/w to about 0.8% w/w, about 0.01% w/w to about 1% w/w, about 0.0108% w/w to about 0.015% w/w, about 0.0108% w/w to about 0.02% w/w, about 0.0108% w/w to about 0.05% w/w, about 0.0108% w/w to about 0.08% w/w, about 0.0108% w/w to about 0.1% w/w, about 0.0108% w/w to about 0.4% w/w, about 0.0108% w/w to about 0.8% w/w, about 0.0108% w/w to about 1% w/w, about 0.015% w/w to about 0.02% w/w, about 0.015% w/w to about 0.05% w/w, about 0.015% w/w to about 0.08% w/w, about 0.015% w/w to about 0.1% w/w, about 0.015% w/w to about 0.4% w/w, about 0.015% w/w to about 0.8% w/w, about 0.015% w/w to about 1% w/w, about 0.02% w/w to about 0.05% w/w, about 0.02% w/w to about 0.08% w/w, about 0.02% w/w to about 0.1% w/w, about 0.02% w/w to about 0.4% w/w, about 0.02% w/w to about 0.8% w/w, about 0.02% w/w to about 1% w/w, about 0.05% w/w to about 0.08% w/w, about 0.05% w/w to about 0.1% w/w, about 0.05% w/w to about 0.4% w/w, about 0.05% w/w to about 0.8% w/w, about 0.05% w/w to about 1% w/w, about 0.08% w/w to about 0.1% w/w, about 0.08% w/w to about 0.4% w/w, about 0.08% w/w to about 0.8% w/w, about 0.08% w/w to about 1% w/w, about 0.1% w/w to about 0.4% w/w, about 0.1% w/w to about 0.8% w/w, about 0.1% w/w to about 1% w/w, about 0.4% w/w to about 0.8% w/w, about 0.4% w/w to about 1% w/w, or about 0.8% w/w to about 1% w/w, including increments therein. In some embodiments, the plant-based honey composition comprises succinic acid in an amount between about 0.001% w/w, about 0.005% w/w, about 0.01% w/w, about 0.0108% w/w, about 0.015% w/w, about 0.02% w/w, about 0.05% w/w, about 0.08% w/w, about 0.1% w/w, about 0.4% w/w, about 0.8% w/w, or about 1% w/w. In some embodiments, the plant-based honey composition comprises succinic acid in an amount between at least about 0.001% w/w, about 0.005% w/w, about 0.01% w/w, about 0.0108% w/w, about 0.015% w/w, about 0.02% w/w, about 0.05% w/w, about 0.08% w/w, about 0.1% w/w, about 0.4% w/w, or about 0.8% w/w. In some embodiments, the plant-based honey composition comprises succinic acid in an amount between at most about 0.005% w/w, about 0.01% w/w, about 0.0108% w/w, about 0.015% w/w, about 0.02% w/w, about 0.05% w/w, about 0.08% w/w, about 0.1% w/w, about 0.4% w/w, about 0.8% w/w, or about 1% w/w. In some embodiments, the plant-based honey composition comprises between 0.01 and 0.0001% w/w succinic acid. In some embodiments, the plant-based honey composition comprises between 0.01 and 0.001% w/w succinic acid. In some embodiments, the plant-based honey composition comprises between 0.001 and 0.0001% w/w succinic acid.

In some embodiments, the non-bee made honey composition comprises gluconic acid in an amount between about 0.01% w/w to about 2% w/w. In some embodiments, the non-bee made honey composition comprises gluconic acid in an amount between about 0.01% w/w to about 0.05% w/w, about 0.01% w/w to about 0.1% w/w, about 0.01% w/w to about 0.13% w/w, about 0.01% w/w to about 0.135% w/w, about 0.01% w/w to about 0.15% w/w, about 0.01% w/w to about 0.5% w/w, about 0.01% w/w to about 0.75% w/w, about 0.01% w/w to about 1% w/w, about 0.01% w/w to about 1.25% w/w, about 0.01% w/w to about 1.75% w/w, about 0.01% w/w to about 2% w/w, about 0.05% w/w to about 0.1% w/w, about 0.05% w/w to about 0.13% w/w, about 0.05% w/w to about 0.135% w/w, about 0.05% w/w to about 0.15% w/w, about 0.05% w/w to about 0.5% w/w, about 0.05% w/w to about 0.75% w/w, about 0.05% w/w to about 1% w/w, about 0.05% w/w to about 1.25% w/w, about 0.05% w/w to about 1.75% w/w, about 0.05% w/w to about 2% w/w, about 0.1% w/w to about 0.13% w/w, about 0.1% w/w to about 0.135% w/w, about 0.1% w/w to about 0.15% w/w, about 0.1% w/w to about 0.5% w/w, about 0.1% w/w to about 0.75% w/w, about 0.1% w/w to about 1% w/w, about 0.1% w/w to about 1.25% w/w, about 0.1% w/w to about 1.75% w/w, about 0.1% w/w to about 2% w/w, about 0.13% w/w to about 0.135% w/w, about 0.13% w/w to about 0.15% w/w, about 0.13% w/w to about 0.5% w/w, about 0.13% w/w to about 0.75% w/w, about 0.13% w/w to about 1% w/w, about 0.13% w/w to about 1.25% w/w, about 0.13% w/w to about 1.75% w/w, about 0.13% w/w to about 2% w/w, about 0.135% w/w to about 0.15% w/w, about 0.135% w/w to about 0.5% w/w, about 0.135% w/w to about 0.75% w/w, about 0.135% w/w to about 1% w/w, about 0.135% w/w to about 1.25% w/w, about 0.135% w/w to about 1.75% w/w, about 0.135% w/w to about 2% w/w, about 0.15% w/w to about 0.5% w/w, about 0.15% w/w to about 0.75% w/w, about 0.15% w/w to about 1% w/w, about 0.15% w/w to about 1.25% w/w, about 0.15% w/w to about 1.75% w/w, about 0.15% w/w to about 2% w/w, about 0.5% w/w to about 0.75% w/w, about 0.5% w/w to about 1% w/w, about 0.5% w/w to about 1.25% w/w, about 0.5% w/w to about 1.75% w/w, about 0.5% w/w to about 2% w/w, about 0.75% w/w to about 1% w/w, about 0.75% w/w to about 1.25% w/w, about 0.75% w/w to about 1.75% w/w, about 0.75% w/w to about 2% w/w, about 1% w/w to about 1.25% w/w, about 1% w/w to about 1.75% w/w, about 1% w/w to about 2% w/w, about 1.25% w/w to about 1.75% w/w, about 1.25% w/w to about 2% w/w, or about 1.75% w/w to about 2% w/w, including increments therein. In some embodiments, the non-bee made honey composition comprises gluconic acid in an amount between about 0.01% w/w, about 0.05% w/w, about 0.1% w/w, about 0.13% w/w, about 0.135% w/w, about 0.15% w/w, about 0.5% w/w, about 0.75% w/w, about 1% w/w, about 1.25% w/w, about 1.75% w/w, or about 2% w/w. In some embodiments, the non-bee made honey composition comprises gluconic acid in an amount between at least about 0.01% w/w, about 0.05% w/w, about 0.1% w/w, about 0.13% w/w, about 0.135% w/w, about 0.15% w/w, about 0.5% w/w, about 0.75% w/w, about 1% w/w, about 1.25% w/w, or about 1.75% w/w. In some embodiments, the non-bee made honey composition comprises gluconic acid in an amount between at most about 0.05% w/w, about 0.1% w/w, about 0.13% w/w, about 0.135% w/w, about 0.15% w/w, about 0.5% w/w, about 0.75% w/w, about 1% w/w, about 1.25% w/w, about 1.75% w/w, or about 2% w/w. In some embodiments, the non-bee made honey composition comprises between 1 and 0.001% w/w gluconic acid. In some embodiments, the non-bee made honey composition comprises between 1 and 0.1% w/w gluconic acid. In some embodiments, the plant-based honey composition comprises gluconic acid in an amount between about 0.01% w/w to about 2% w/w. In some embodiments, the plant-based honey composition comprises gluconic acid in an amount between about 0.01% w/w to about 0.05% w/w, about 0.01% w/w to about 0.1% w/w, about 0.01% w/w to about 0.13% w/w, about 0.01% w/w to about 0.135% w/w, about 0.01% w/w to about 0.15% w/w, about 0.01% w/w to about 0.5% w/w, about 0.01% w/w to about 0.75% w/w, about 0.01% w/w to about 1% w/w, about 0.01% w/w to about 1.25% w/w, about 0.01% w/w to about 1.75% w/w, about 0.01% w/w to about 2% w/w, about 0.05% w/w to about 0.1% w/w, about 0.05% w/w to about 0.13% w/w, about 0.05% w/w to about 0.135% w/w, about 0.05% w/w to about 0.15% w/w, about 0.05% w/w to about 0.5% w/w, about 0.05% w/w to about 0.75% w/w, about 0.05% w/w to about 1% w/w, about 0.05% w/w to about 1.25% w/w, about 0.05% w/w to about 1.75% w/w, about 0.05% w/w to about 2% w/w, about 0.1% w/w to about 0.13% w/w, about 0.1% w/w to about 0.135% w/w, about 0.1% w/w to about 0.15% w/w, about 0.1% w/w to about 0.5% w/w, about 0.1% w/w to about 0.75% w/w, about 0.1% w/w to about 1% w/w, about 0.1% w/w to about 1.25% w/w, about 0.1% w/w to about 1.75% w/w, about 0.1% w/w to about 2% w/w, about 0.13% w/w to about 0.135% w/w, about 0.13% w/w to about 0.15% w/w, about 0.13% w/w to about 0.5% w/w, about 0.13% w/w to about 0.75% w/w, about 0.13% w/w to about 1% w/w, about 0.13% w/w to about 1.25% w/w, about 0.13% w/w to about 1.75% w/w, about 0.13% w/w to about 2% w/w, about 0.135% w/w to about 0.15% w/w, about 0.135% w/w to about 0.5% w/w, about 0.135% w/w to about 0.75% w/w, about 0.135% w/w to about 1% w/w, about 0.135% w/w to about 1.25% w/w, about 0.135% w/w to about 1.75% w/w, about 0.135% w/w to about 2% w/w, about 0.15% w/w to about 0.5% w/w, about 0.15% w/w to about 0.75% w/w, about 0.15% w/w to about 1% w/w, about 0.15% w/w to about 1.25% w/w, about 0.15% w/w to about 1.75% w/w, about 0.15% w/w to about 2% w/w, about 0.5% w/w to about 0.75% w/w, about 0.5% w/w to about 1% w/w, about 0.5% w/w to about 1.25% w/w, about 0.5% w/w to about 1.75% w/w, about 0.5% w/w to about 2% w/w, about 0.75% w/w to about 1% w/w, about 0.75% w/w to about 1.25% w/w, about 0.75% w/w to about 1.75% w/w, about 0.75% w/w to about 2% w/w, about 1% w/w to about 1.25% w/w, about 1% w/w to about 1.75% w/w, about 1% w/w to about 2% w/w, about 1.25% w/w to about 1.75% w/w, about 1.25% w/w to about 2% w/w, or about 1.75% w/w to about 2% w/w, including increments therein. In some embodiments, the plant-based honey composition comprises gluconic acid in an amount between about 0.01% w/w, about 0.05% w/w, about 0.1% w/w, about 0.13% w/w, about 0.135% w/w, about 0.15% w/w, about 0.5% w/w, about 0.75% w/w, about 1% w/w, about 1.25% w/w, about 1.75% w/w, or about 2% w/w. In some embodiments, the plant-based honey composition comprises gluconic acid in an amount between at least about 0.01% w/w, about 0.05% w/w, about 0.1% w/w, about 0.13% w/w, about 0.135% w/w, about 0.15% w/w, about 0.5% w/w, about 0.75% w/w, about 1% w/w, about 1.25% w/w, or about 1.75% w/w. In some embodiments, the plant-based honey composition comprises gluconic acid in an amount between at most about 0.05% w/w, about 0.1% w/w, about 0.13% w/w, about 0.135% w/w, about 0.15% w/w, about 0.5% w/w, about 0.75% w/w, about 1% w/w, about 1.25% w/w, about 1.75% w/w, or about 2% w/w. In some embodiments, the plant-based honey composition comprises between 1 and 0.001% w/w gluconic acid. In some embodiments, the plant-based honey composition comprises between 1 and 0.1% w/w gluconic acid.

### Polyphenols

Honeys may contain a range of polyphenols, compounds containing more than one phenolic hydroxyl group. These compounds comprise a vast family containing more than 8,000 polyphenols identified to date, all of which are derived from plants. As such, polyphenols often make up some fraction of all honeys found in nature. The specific polyphenols added to a honey depend on the plant or floral origin of the nectar and therefore can be selected based on analysis of specific honeys, either based on plants of origin (clover, wildflower, orange, etc.) or based on analysis such as high performance liquid chromatography (HPLC) or mass spectroscopy (MS). These compounds are instrumental in distinguishing honey from other sweet syrup-like substances as a superfood.

Simple phenols are those with a C6 carbon structure (such as phenol itself, cresol and thymol). Some have a C6-C1 structure, such as phenolic acids (for example, gallic, vanillic and syringic acids) and aldehydes (for example, vanillin). Those with a C6-C2 structure include phenylacetic acids and acetophenones, and the phenylpropanoid group with a C6-C3 structure are mainly represented by the hydroxycinnamic acids such as p-coumaric, ferulic and caffeic acids and their respective derivatives. Simple phenols, phenolic and phenylacetic acids can be found free in a large variety of plant species but their methyl and ethyl esters and glycosides can also occur in the free form and/or bound forms. Flavonoids with a C6-C3-C6 structure represent the most widely distributed phenolic compounds in plants, and their carbon skeleton consists of an aromatic ring fused to a heterocyclic ring (C) carrying an oxygen atom, and the other ring generally linked to number two carbon atom of the ring C. In nature, they can be found as free aglycones although they usually occur as glycosides and their derivatives. Flavonoids are further divided and classified with respect to the degree of oxidation of the ring C into flavones such as apigenin, luteolin and diosmetin, flavonols such as quercetin, myricetin and kaempferol, flavanones such as naringenin and hesperidin, flavan-3-ols such as catechin and epicatechin, anthocyanidins and their glycosides such as malvidin, cyaniding and pelargonidin. It is important to mention that flavones, flavonols and their glycosides are the most common flavonoids found in the plant kingdom. Tannins are molecules of intermediate to high molecular weight and have an important degree of hydroxylation. They are typically found in plants and can be classified in two large groups: hydrolysable tannins and condensed tannins; although there is a third group, the phlorotannins, which occurs in brown algae but are not consumed by humans. Hydrolysable tannins consist of molecules of either gallic acid or ellagic acid esterified to a polyol molecule such as glucose. These tannins can increase their molecular weight by consecutive esterification with other galloyl or ellagic molecules forming large polymers.

Studies into the biochemistry of polyphenols and their properties of pharmacological and therapeutic interest have been extensive. For example, flavonoids have important benefits in cardiovascular health, mainly on blood pressure and in the prevention of the damage due to lipid peroxidation of the low density proteins. Other therapeutic aspects of polyphenols are the protection of gastric mucus, liver mucus, anti-inflammatory and anti-neoplasic behavior, antimicrobial activity (including the selective inhibition of some immunodeficiency viruses such as HIV-1 and HIV-2. Polyphenols can behave as antioxidants because they function as terminators of free radicals and chelators of metal ions which could catalyze the lipid peroxidation. The antioxidant activity of these molecules is due to a rapid donation of hydrogen atoms to stabilize the free radicals and the remaining phenoxy radicals can also react with any other radical to terminate the propagation route. The chemical structure of polyphenols greatly influences their antioxidant activity. Flavonoids, have one or more of some structural features involved in antioxidant activity such as: an odiphenolic group in the B ring, a 2-3 double bond conjugated with the 4-oxo function (in ring C), hydroxyl groups in positions 5 and 7 (in ring A). The specific phenolic compounds can be identified in different types of honey using either high-performance liquid chromatography (HPLC) or capillary electrophoresis (CE) techniques with diode array detection (DAD) (Pyrzynska and Biesaga, J Chromatogr 1216(38):6620-6 (2009) Generally, the antioxidant activity also correlates directly with the degree of hydroxylation (Ratty and Das, Biochem Pharmacol. 37(6):989-995 (1988). In nature, flavonoids usually occur as 3-O-glycosides and the presence of the sugar moiety dramatically decreases the antioxidant activity of the compound. Yet, free flavonoids, best known as aglycones, have known antioxidant activity. The role of polyphenols as antioxidant agents against radical reactive species such as those belonging to the reactive oxygen species (ROS) and reactive nitrogen species (RNS) groups is of remarkable interest. Radicals belonging to ROS such as OH (hydroxyl), RCOO (peroxyl) and RO (alcohoxyl) are of major concern in the study of the oxidative stress of living tissues since they can be scavenged by flavonoids. On the other hand, the highly reactive nitrogen species peroxynitrite (ONOO—) has been investigated regarding the ability of flavonoids, such as anthocyanins, to act as scavengers. Investigation of the relationship between the chemical structure and the antioxidant power (in terms of radical scavenging activity) of several flavonoids demonstrated that this activity strongly depends on the substitution pattern of the free hydroxyl groups in its base structure. Uthurry, et al. J. ApiProduct ApiMedical Science 3 (4): 141-159 (2011). Many such phenolic and flavonoid compounds, including quercetin, kaempferol, apigenin, and caffeic acid, have antioxidant and anti-platelet potential, and hence may ameliorate cardiovascular diseases (CVDs) through various mechanisms, such as by decreasing oxidative stress and inhibiting blood platelet activation. Beata Olas, Nutrients 12(2): 283 (2020).

Many of the polyphenolic class of compounds include organic acids and flavonoids (flavones and flavonols), that are also found in bee-made honeys. Representative compounds also include proanthocyanidins, including catechin and epicatechin. These compounds contribute to the base formulation in several ways. The first way is that they are bioactive compounds that potentiate health benefits to the consumer. These compounds are instrumental in distinguishing honey from other sweet syrup-like substances as a superfood.

The second contribution of these compounds is color. Polyphenols range in color from colorless, to yellow, brown, red etc. Because of this, their use can contribute to honey-color without the need for adding colorants to the composition.

The third contribution of polyphenols is to taste. Organic acids can be added to lower pH, and range in taste from tart to sour, sharp to gradual. Many flavonoids can also impart texture and taste, ranging from chalky to powdery, tannic to floral. Their taste is more delicate but contributes to the subtle complexity of the flavor profile none-the-less. Proanthocyanidins can add a tannic and astringent nature to food and can contribute these profiles to honey as well.

Plant-based honey may contain unique blends of polyphenols that emulate the specific composition of a monofloral honey variety. Polyphenols of interest include the following: quercetin, galangin, hesperetin, chrysin, pinocembrin, naringenin, pinobanksin, luteolin, apigenin, myricetin, rutin, and kaempferol. In some embodiments, the non-bee made honey comprises a category of polyphenols such as flavonoids, phenolic acids, C-glycosylflavones, coumarins, isoflavones, sesquiterpenes, triterpenes, catechins, flavanols, flavones, procyanidins, proanthocyanidin, quinones, theaflavins, thearubigins, or stilbenes. In some embodiments, the non-bee made honey formulation comprises one or more polyphenols including quercetin, galangin, hesperetin, chrysin, pinocembrin, naringenin, pinobanksin, luteolin, apigenin, genistein, catechin, epicatechin, isorhamnetic, myricetin, rutin, kaempferol, and derivatives thereof. In some embodiments, the non-bee made honey comprises a polyphenol or a or a glycosylated derivative thereof. In some embodiments, the plant based honey comprises a category of polyphenols such as flavonoids, phenolic acids, C-glycosylflavones, coumarins, isoflavones, sesquiterpenes, triterpenes, catechins, flavanols, flavones, procyanidins, proanthocyanidin, quinones, theaflavins, thearubigins, or stilbenes. In some embodiments, the plant based honey comprises a polyphenol or a or a glycosylated derivative thereof.

In some embodiments, the plant based honey comprises quercetin. In some embodiments, the plant-based honey comprises apigenin. In some embodiments, the plant based honey comprises luteolin. In some embodiments, the plant based honey comprises kaempferol. In some embodiments, the plant based honey comprises isovitexin. In some embodiments, the plant based honey comprises chrysin. In some embodiments, the plant based honey comprises orientin. In some embodiments, the plant based honey comprises vitexin. In some embodiments, the plant based honey comprises isoorientin. In some embodiments, the plant based honey comprises scopoletin. In some embodiments, the plant based honey comprises umbelliferone. In some embodiments, the plant based honey comprises chlorogenic acid. In some embodiments, the plant based honey comprises caffeic acid. In some embodiments, the plant based honey comprises ferulic acid. In some embodiments, the plant based honey comprises formoneith. In some embodiments, the plant based honey comprises biochanin A. In some embodiments, the plant based honey comprises daidzein. In some embodiments, the plant based honey comprises genistein. In some embodiments, the plant based honey comprises coumestrol. In some embodiments, the plant based honey comprises medicagol. In some embodiments, the plant based honey comprises p-coumaric acid. In some embodiments, the plant based honey comprises p-hydroxybenzoic acid. In some embodiments, the plant based honey comprises herniarin. In some embodiments, the plant based honey comprises α-bisabolol. α-bisabolol. In some embodiments, the plant based honey comprises chamazuelene. In some embodiments, the plant based honey comprises rosmarinic acid. In some embodiments, the plant based honey comprises salvianolic acid. In some embodiments, the plant based honey comprises ursolic acid. In some embodiments, the plant based honey comprises oleanolic acid. In some embodiments, the plant based honey comprises epicatechin. In some embodiments, the plant based honey comprises epicatechin gallate. In some embodiments, the plant based honey comprises epigallocatechin. In some embodiments, the plant based honey comprises epigallocatechin gallate. In some embodiments, the plant based honey comprises gallic acid. In some embodiments, the plant based honey comprises isorhamnetin. In some embodiments, the plant based honey comprises ellagic acid. In some embodiments, the plant based honey comprises ubiquinone. In some embodiments, the plant based honey comprises α-tocopherolquinone. In some embodiments, the plant based honey comprises theaflavin. In some embodiments, the plant based honey comprises thearubigin. In some embodiments, the plant based honey comprises myricetin. In some embodiments, the plant based honey comprises resveratrol. In some embodiments, the plant based honey comprises protocatechuic acid.

There are no colorants that are not natural from the ingredients used nor thickeners such as xanthum gum or proteins in the formulation disclosed herein. Color, physical characteristics, flavor and fragrance is provided by the polyphenols discussed above.

Inclusion of polyphenols sourced directly from plants in sugar syrups or honey alternative products has remained a challenging approach for a number of reasons, including but not limited to: the lack of guidance in the art as to selection of particular polyphenols, difficultly in characterizing the properties (e.g., taste, color, smell, health benefits, antioxidant activity, etc.) of the polyphenol and to what extent those properties are contributed to the final product comprising the polyphenol, difficulty in predicting how various polyphenol compounds may interact, look, taste, or smell when mixed in solution, among other challenges associated with the use of polyphenol in the production of non-bee made honey formulation. Polyphenols may exhibit antioxidant activity and may contribute these properties to the non-bee made honey formulation. They may also function at phytochemicals—which are powerful bioactive plant compounds known for their roles in helping plants resist bacterial and viral infections. Their benefits are similarly recorded in humans. Recently, the effects of polyphenols in preventing coronavirus infection have also been noted [Russo M, et al. Chem Biol Interact. 2020 Sep. 1; 328:109211. doi: 10.1016/j.cbi.2020.109211. Epub 2020 Jul. 28. PMID: 32735799; PMCID: PMC7385538.]

Honey can contain a plethora of polyphenols further subcategorized at organic acids and flavonoids, all of which can contribute to the taste, texture, color, aroma, mouthfeel, and other characteristics of the product, and choosing particular polyphenols and in what quantity sufficient to produce a non-bee made honey formulation which closely approximates bee-made honey remains a challenge. In some embodiments, the non-bee made honey comprises one or more polyphenols including kaempferol, quercetin, and/or isorhamnetin.

In some embodiments, the one or more polyphenols are present in an amount of about 0.1% to about 4%. In some embodiments, the one or more polyphenols are present in an amount of about 0.1% to about 0.4%, about 0.1% to about 0.6%, about 0.1% to about 0.8%, about 0.1% to about 1%, about 0.1% to about 1.4%, about 0.1% to about 1.8%, about 0.1% to about 2.4%, about 0.1% to about 2.8%, about 0.1% to about 3.2%, about 0.1% to about 3.6%, about 0.1% to about 4%, about 0.4% to about 0.6%, about 0.4% to about 0.8%, about 0.4% to about 1%, about 0.4% to about 1.4%, about 0.4% to about 1.8%, about 0.4% to about 2.4%, about 0.4% to about 2.8%, about 0.4% to about 3.2%, about 0.4% to about 3.6%, about 0.4% to about 4%, about 0.6% to about 0.8%, about 0.6% to about 1%, about 0.6% to about 1.4%, about 0.6% to about 1.8%, about 0.6% to about 2.4%, about 0.6% to about 2.8%, about 0.6% to about 3.2%, about 0.6% to about 3.6%, about 0.6% to about 4%, about 0.8% to about 1% about 0.8% to about 1.4%, about 0.8% to about 1.8%, about 0.8% to about 2.4%, about 0.8% to about 2.8%, about 0.8% to about 3.2%, about 0.8% to about 3.6%, about 0.8% to about 4%, about 1% to about 1.4%, about 1% to about 1.8%, about 1% to about 2.4%, about 1% to about 2.8%, about 1% to about 3.2%, about 1% to about 3.6%, about 1% to about 4%, about 1.4% to about 1.8%, about 1.4% to about 2.4%, about 1.4% to about 2.8%, about 1.4% to about 3.2%, about 1.4% to about 3.6%, about 1.4% to about 4%, about 1.8% to about 2.4%, about 1.8% to about 2.8%, about 1.8% to about 3.2%, about 1.8% to about 3.6%, about 1.8% to about 4%, about 2.4% to about 2.8%, about 2.4% to about 3.2%, about 2.4% to about 3.6%, about 2.4% to about 4%, about 2.8% to about 3.2%, about 2.8% to about 3.6%, about 2.8% to about 4%, about 3.2% to about 3.6%, about 3.2% to about 4%, or about 3.6% to about 4%, including increments therein. In some embodiments, the one or more polyphenols are present in an amount of about 0.1%, about 0.4%, about 0.6%, about 0.8%, about 1% about 1.4%, about 1.8%, about 2.4%, about 2.8%, about 3.2%, about 3.6%, or about 4%. In some embodiments, the one or more polyphenols are present in an amount of at least about 0.1%, about 0.4%, about 0.6%, about 0.8%, about 1%, about 1.4%, about 1.8%, about 2.4%, about 2.8%, about 3.2%, or about 3.6%. In some embodiments, the one or more polyphenols are present in an amount of at most about 0.4%, about 0.6%, about 0.8%, about 1%, about 1.4%, about 1.8%, about 2.4%, about 2.8%, about 3.2%, about 3.6%, or about 4% w/w. In some embodiments, the one or more polyphenols are present in an amount of at least about 0.1%, at least about 0.4%, at least about 0.6%, at least about 0.8%, at least about 1%, at least about 1.4%, at least about 1.8%, at least about 2.4%, at least about 2.8%, at least about 3.2%, or at least about 3.6%. In some embodiments, the one or more polyphenols are present in an amount of at most about 0.4%, at most about 0.6%, at most about 0.8%, at most about 1%, at most about 1.4%, at most about 1.8%, at most about 2.4%, at most about 2.8%, at most about 3.2%, at most about 3.6%, or at most about 4% w/w.

In some embodiments, the plant-based honey comprises one or more polyphenols that are present in an amount of about 0.1% to about 4%. In some embodiments, the one or more polyphenols are present in an amount of about 0.1% to about 0.4%, about 0.1% to about 0.6%, about 0.1% to about 0.8%, about 0.1% to about 1%, about 0.1% to about 1.4%, about 0.1% to about 1.8%, about 0.1% to about 2.4%, about 0.1% to about 2.8%, about 0.1% to about 3.2%, about 0.1% to about 3.6%, about 0.1% to about 4%, about 0.4% to about 0.6%, about 0.4% to about 0.8%, about 0.4% to about 1%, about 0.4% to about 1.4%, about 0.4% to about 1.8%, about 0.4% to about 2.4%, about 0.4% to about 2.8%, about 0.4% to about 3.2%, about 0.4% to about 3.6%, about 0.4% to about 4%, about 0.6% to about 0.8%, about 0.6% to about 1%, about 0.6% to about 1.4%, about 0.6% to about 1.8%, about 0.6% to about 2.4%, about 0.6% to about 2.8%, about 0.6% to about 3.2%, about 0.6% to about 3.6%, about 0.6% to about 4%, about 0.8% to about 1%, about 0.8% to about 1.4%, about 0.8% to about 1.8%, about 0.8% to about 2.4%, about 0.8% to about 2.8%, about 0.8% to about 3.2%, about 0.8% to about 3.6%, about 0.8% to about 4%, about 1% to about 1.4%, about 1% to about 1.8%, about 1% to about 2.4%, about 1% to about 2.8%, about 1% to about 3.2%, about 1% to about 3.6%, about 1% to about 4%, about 1.4% to about 1.8%, about 1.4% to about 2.4%, about 1.4% to about 2.8%, about 1.4% to about 3.2%, about 1.4% to about 3.6%, about 1.4% to about 4%, about 1.8% to about 2.4%, about 1.8% to about 2.8%, about 1.8% to about 3.2%, about 1.8% to about 3.6%, about 1.8% to about 4%, about 2.4% to about 2.8%, about 2.4% to about 3.2%, about 2.4% to about 3.6%, about 2.4% to about 4%, about 2.8% to about 3.2%, about 2.8% to about 3.6%, about 2.8% to about 4%, about 3.2% to about 3.6%, about 3.2% to about 4%, or about 3.6% to about 4%, including increments therein. In some embodiments, the one or more polyphenols are present in an amount of about 0.1%, about 0.4%, about 0.6%, about 0.8%, about 1%, about 1.4%, about 1.8%, about 2.4%, about 2.8%, about 3.2%, about 3.6%, or about 4%. In some embodiments, the one or more polyphenols are present in an amount of at least about 0.1%, about 0.4%, about 0.6%, about 0.8%, about 1%, about 1.4%, about 1.8%, about 2.4%, about 2.8%, about 3.2%, or about 3.6%. In some embodiments, the one or more polyphenols are present in an amount of at most about 0.4%, about 0.6%, about 0.8%, about 1%, about 1.4%, about 1.8%, about 2.4%, about 2.8%, about 3.2%, about 3.6%, or about 4% w/w. In some embodiments, the one or more polyphenols are present in an amount of at least about 0.1%, at least about 0.4%, at least about 0.6%, at least about 0.8%, at least about 1%, at least about 1.4%, at least about 1.8%, at least about 2.4%, at least about 2.8%, at least about 3.2%, or at least about 3.6%. In some embodiments, the one or more polyphenols are present in an amount of at most about 0.4%, at most about 0.6%, at most about 0.8%, at most about 1%, at most about 1.4%, at most about 1.8%, at most about 2.4%, at most about 2.8%, at most about 3.2%, at most about 3.6%, or at most about 4% w/w.

In some embodiments, the plant-based honey comprises one or more polyphenols that are present in a concentration of about 0.1 mM to about 1 mM. In some embodiments, the one or more polyphenols are present in a concentration of about 0.1 mM to about 0.4 mM, about 0.1 mM to about 0.6 mM, about 0.1 mM to about 0.8 mM, about 0.1 mM to about 1 mM, about 0.1 mM to about 1.4 mM, about 0.1 mM to about 1.8 mM, about 0.1 mM to about 2.4 mM, about 0.1 mM to about 2.8 mM, about 0.1 mM to about 3.2 mM, about 0.1 mM to about 3.6 mM, about 0.1 mM to about 4 mM, about 0.4 mM to about 0.6 mM, about 0.4 mM to about 0.8 mM, about 0.4 mM to about 1 mM, about 0.4 mM to about 1.4 mM, about 0.4 mM to about 1.8 mM, about 0.4 mM to about 2.4 mM, about 0.4 mM to about 2.8 mM, about 0.4 mM to about 3.2 mM, about 0.4 mM to about 3.6 mM, about 0.4 mM to about 4 mM, about 0.6 mM to about 0.8 mM, about 0.6 mM to about 1 mM, about 0.6 mM to about 1.4 mM, about 0.6 mM to about 1.8 mM, about 0.6 mM to about 2.4 mM, about 0.6 mM to about 2.8 mM, about 0.6 mM to about 3.2 mM, about 0.6 mM to about 3.6 mM, about 0.6 mM to about 4 mM, about 0.8 mM to about 1 mM, about 0.8 mM to about 1.4 mM, about 0.8 mM to about 1.8 mM, about 0.8 mM to about 2.4 mM, about 0.8 mM to about 2.8 mM, about 0.8 mM to about 3.2 mM, about 0.8 mM to about 3.6 mM, about 0.8 mM to about 4 mM, about 1 mM to about 1.4 mM, about 1 mM to about 1.8 mM, about 1 mM to about 2.4 mM, about 1 mM to about 2.8 mM, about 1 mM to about 3.2 mM, about 1 mM to about 3.6 mM, about 1 mM to about 4 mM, about 1.4 mM to about 1.8 mM, about 1.4 mM to about 2.4 mM, about 1.4 mM to about 2.8 mM, about 1.4 mM to about 3.2 mM, about 1.4 mM to about 3.6 mM, about 1.4 mM to about 4 mM, about 1.8 mM to about 2.4 mM, about 1.8 mM to about 2.8 mM, about 1.8 mM to about 3.2 mM, about 1.8 mM to about 3.6 mM, about 1.8 mM to about 4 mM, about 2.4 mM to about 2.8 mM, about 2.4 mM to about 3.2 mM, about 2.4 mM to about 3.6 mM, about 2.4 mM to about 4 mM, about 2.8 mM to about 3.2 mM, about 2.8 mM to about 3.6 mM, about 2.8 mM to about 4 mM, about 3.2 mM to about 3.6 mM, about 3.2 mM to about 4 mM, or about 3.6 mM to about 4 mM, including increments therein. In some embodiments, the one or more polyphenols are present in a concentration of about 0.1 mM, about 1 mM, about 0.6 mM, about 0.8 mM, about 1 mM, about 1.4 mM, about 1.8 mM, about 2.4 mM, about 2.8 mM, about 3.2 mM, about 3.6 mM, or about 4 mM. In some embodiments, the one or more polyphenols are present in a concentration of at least about 0.1 mM, about 0.4 mM, about 0.6 mM, about 0.8 mM, about 1 mM, about 1.4 mM, about 1.8 mM, about 2.4 mM, about 2.8 mM, about 3.2 mM, or about 3.6 mM. In some embodiments, the one or more polyphenols are present in a concentration of at most about 0.4 mM, about 0.6 mM, about 0.8 mM, about 1 mM, about 1.4 mM, about 1.8 mM, about 2.4 mM, about 2.8 mM, about 3.2 mM, about 3.6 mM, or about 4 mM. In some embodiments, the one or more polyphenols are present in a concentration of at least about 0.1 mM, at least about 0.4 mM, at least about 0.6 mM, at least about 0.8 mM, at least about 1 mM, at least about 1.4 mM, at least about 1.8 mM, at least about 2.4 mM, at least about 2.8 mM, at least about 3.2 mM, or at least about 3.6 mM. In some embodiments, the one or more polyphenols are present in a concentration of at most about 0.4 mM, at most about 0.6 mM, at most about 0.8 mM, at most about 1 mM, at most about 1.4 mM, at most about 1.8 mM, at most about 2.4 mM, at most about 2.8 mM, at most about 3.2 mM, at most about 3.6 mM, or at most about 4 mM w/w. In some embodiments, the one or more polyphenols are present in a concentration of about 0.01 mM to about 0.1 mM. In some embodiments, the one or more polyphenols are present in a concentration of about 0.001 mM to about 0.1 mM. In some embodiments, the one or more polyphenols are present in a concentration of about 0.0001 mM to about 0.1 mM. In some embodiments, the one or more polyphenols are present in a concentration of about 0.0001 mM to about 1.0 mM. In some embodiments, the one or more polyphenols are present in a concentration of about 0.001 mM to about 1.0 mM. In some embodiments, the one or more polyphenols are present in a concentration of about 0.01 mM to about 1.0 mM. In some embodiments, the one or more polyphenols are present in a concentration of about 0.0001 mM to about 10.0 mM. In some embodiments, the one or more polyphenols are present in a concentration of about 0.001 mM to about 10.0 mM. In some embodiments, the one or more polyphenols are present in a concentration of about 0.01 mM to about 10.0 mM. In some embodiments, the one or more polyphenols are present in a concentration of about 0.1 mM to about 10.0 mM. In some embodiments, the one or more polyphenols are present in a concentration of about 0.1 mM to about 100.0 mM. In some embodiments, the one or more polyphenols are present in a concentration of about 0.1 mM to about 1.0 M. In some embodiments, the one or more polyphenols are present in a concentration of about 0.01 mM to about 1.0 M. In some embodiments, the one or more polyphenols are present in a concentration of about 0.001 mM to about 1.0 M. In some embodiments, the one or more polyphenols are present in a concentration of about 0.1 mM to about 10.0 M. In some embodiments, the one or more polyphenols are present in a concentration of about 0.1 mM to about 100.0 M.

Exemplary compounds comprised within the formulations described herein may include:

Gallic acid—an organic acid found in several plant species, and in some honeys, known in the literature as being effective in preventing oxidative DNA damage and reducing markers characteristic of increased risk of developing cancer [Ferk F, et al Mol Nutr Food Res. 2018 February; 62(4). doi: 10.1002/mnfr.201700482. Epub 2018 Jan. 15. PMID: 29193677]. Gallic acid has also been demonstrated to have both anti-inflammatory and anti-obesity capabilities, as demonstrated by pre-clinical data [Dludla et al. Nutrients. 2018 Dec. 21; 11(1):23. doi: 10.3390/nu11010023. PMID: 30577684; PMCID: 5 PMC6356415.]

Chlorogenic acid—A phenolic compound and organic acid of the hydroxycinnamic acid family, chlorogenic acid is found in many plants and known to be present in green coffee beans in high amounts. It possesses several health benefits and has been the subject of extensive research. As a food additive, chlorogenic acid has anti-microbial activity against a range of microorganisms and it also prevents the degradation of other bioactive compounds. It has also been documented as a nutraceutical with anti-oxidant, antihypertensive, antidiabetic, anti-inflammatory, and anti-carcinogenic properties. It can further function as a prebiotic. [Santana-Gálvez J, et al. Molecules. 2017 Feb. 26; 22(3):358. doi: 10.3390/molecules22030358. PMID: 28245635; PMCID: PMC6155416; Tajik N, et al. Eur J Nutr. 2017 October; 56(7):2215-2244. doi: 10.1007/s00394-017-1379-1. Epub 2017 Apr. 8. PMID: 28391515.]

Quercetin—A polyphenolic compound, specifically a flavonol, found commonly in may plants and flowers of plants. Quercetin is broadly acting and has been implicated as regulating blood-pressure and mood regulation, while possessing potent anti-carcinogenic and anti-inflammatory effects. Its multifaceted functional properties potentiate its role as an anti-aging treatment for its ability to prevent oxidative damage. It is highly active at a free-radical scavenger capable of neutralizing reactive species such as peroxynitrie. [D'Andrea G. Fitoterapia. 2015 October; 106:256-71. doi: 10.1016/j.fitote.2015.09.018. Epub 2015 Sep. 21. PMID: 26393898; et al. Eur J Pharmacol. 2008 May 13; 585(2-3):325-37. doi: 10.1016/j.ejphar.2008.03.008. Epub 2008 Mar. 18. PMID: 18417116.]. In some embodiments, the non-bee made honey comprises quercetin in an amount greater that 0.038% w/w. In some embodiments, the non-bee made honey formulation comprises quercetin in an amount from about 0.04% to about 0.10% w/w. In some embodiments, the non-bee made honey formulation comprises quercetin in an amount of about 0.05% w/w.

Chrysin—Another polyphenolic compound and flavonoid, though of the subclass flavone, chrysin is found most frequently in passionflower, propolis, and honey. The therapeutic effects of chrysin are many and have been well documented. It possesses neuroprotective effects and suppresses neuroinflammation, in addition to providing anti-amyloidogenic and neuroprotective effects. As such, chrysin prevents cognitive decline, and has been reported to help with depression and epilepsy. [Nabavi S F, et al. Neurochem Int. 2015 November; 90:224-31. doi: 10.1016/j.neuint.2015.09.006. Epub 2015 Sep. 18. PMID: 26386393.] Chrysin also provides similar anti-carcinogenic and anti-inflammatory effects as other polyphenols and helps reduce cytotoxicity through its action at an antioxidant. [Mani R, et al. Phytochemistry. 2018 January; 145:187-196. doi: 10.1016/j.phytochem.2017.09.016. Epub 2017 Nov. 20. PMID: 29161583; Naz S, et al. Life Sci. 2019 Oct. 15; 235:116797. doi: 10.1016/j.lfs.2019.116797. Epub 2019 Aug. 28. PMID: 31472146.]

Apigenin—A flavonoid of the flavone subclass, apigenin is known as the main health-promoting component of chamomile and displays potent antioxidant activity. Apigenin has been the subject of several human studies and has shown promise in treatment and/or prevention of insomnia, anxiety, cancer, depression, and even Alzheimer's disease. It is also regarded as a treatment for inflammation of the central nervous system in disease manifestations like multiple sclerosis. [Salehi B, et al., Int J Mol Sci. 2019 Mar. 15; 20(6):1305. doi: 10.3390/ijms20061305. PMID: 30875872; PMCID: PMC6472148; Imran M, et al. Phytother Res. 2020 August; 34(8):1812-1828. doi: 10.1002/ptr.6647. Epub 2020 Feb. 14. PMID: 32059077.]

Luteolin—Luteolin is another flavone that displays strong antioxidant activity. In addition to anti-inflammatory and general anti-carcinogenic effects, luteolin has been shown to inhibit the survival of human bladder cancer cells by upregulating p21 and inhibiting rampant mTOR signaling. [Naiki T, et al. Cancer Sci. 2020 April; 111(4):1165-1179.] Luteolin has also been shown to improve central nervous system disorders by regulating several pathways. [Nabavi S F, et al. Brain Res Bull. 2015 October; 119(Pt A):1-11. doi: 10.1016/j.brainresbull.2015.09.002. Epub 2015 Sep. 8. PMID: 26361743.; Ashaari Z, et al. J Mol Neurosci. 2018 August; 65(4):491-506. doi: 10.1007/s12031-018-1094-2. Epub 2018 Aug. 6. PMID: 30083786.]

Kaempferol—A flavonol with potent anti-oxidant activity and broadly acting as a therapeutic agent in diseases such as diabetes, asthma, microbial contamination disorders, obesity, cardiovascular disease to name a few [Imran M, et al. Phytother Res. 2019 February; 33(2):263-275. doi: 10.1002/ptr.6227. Epub 2018 Nov. 7. PMID: 30402931.] Kaempferol has been found to attenuate liver fibrosis, specifically through its ability to inhibit the activin receptor-like kinase 5. [Xu T, et al., J Cell Mol Med. 2019 September; 23(9):6403-6410. doi: 10.1111/jcmm.14528. Epub 2019 Jul. 5. PMID: 31273920; PMCID: PMC6714241.] The activity of kaempferol in cancer models has also been investigated, showing that kaempferol could modulate apoptosis, angiogenesis, and metastasis of cancerous cell mass. Importantly, it was shown that kaempferol did not negatively affect normal host cells, and even displays an overall protective effect. [Chen et al., Food Chem. 2013 Jun. 15; 138(4):2099-107. doi: 10.1016/j.foodchem.2012.11.139. Epub 2012 Dec. 28. PMID: 23497863; PMCID: PMC3601579.]

In some embodiments, the non-bee made honey comprises one or more proanthocyanidins. In some embodiments, the one or more proanthocyanidins are present in an amount up to about 0.2 mg per 30 mL of the formulation. In some embodiments, the one or more proanthocyanidins are present in an amount up to about 0.02 mg per 30 mL of the formulation. In some embodiments, the one or more proanthocyanidins are present in an amount of at least 0.0001%. In some embodiments, the one or more proanthocyanidins are present in an amount of 0.0001% to about 0.02% w/w. In some embodiments, the one or more proanthocyanidins are present in an amount of 0.00010% to about 0.2% w/w. In some embodiments, the one or more proanthocyanidins are present in an amount of 0.00001% to about 0.001% w/w. In some embodiments, the one or more proanthocyanidins are present in an amount of 0.0001% to about 0.002% w/w. In some embodiments, the non-bee made honey comprises one or more proanthocyanidins in an amount from 0.0001% to about 0.1% w/w. In some embodiments, the non-bee made honey comprises one or more proanthocyanidins in an amount from about 5 mg to about 20 mg per 30 mL of the formulation. In some embodiments, the non-bee made honey comprises one or more proanthocyanidins in an amount from 0.0001% to about 0.001% w/w. In some embodiments, the non-bee made honey comprises one or more proanthocyanidins in an amount from 0.0001% to about 0.10% w/w. In some embodiments, the non-bee made honey comprises one or more proanthocyanidins in an amount from about 5 mg to about 20 mg per 30 mL of the formulation. In some embodiments, the non-bee made honey comprises one or more proanthocyanidins in an amount from 0.0001% to about 0.001% w/w. In some embodiments, the non-bee made honey comprises one or more proanthocyanidins in an amount from 0.0001% to about 0.1% w/w. In some embodiments, the non-bee made honey comprises one or more proanthocyanidins in an amount from about 5 mg to about 20 mg per 30 mL of the non-bee made honey formulation. In some embodiments, the non-bee made honey comprises one or more proanthocyanidins in an amount from about 1 mg to about 20 mg per 30 mL of the non-bee made honey formulation. In some embodiments, the non-bee made honey comprises one or more proanthocyanidins in an amount from about 10 mg to about 20 mg per 30 mL of the non-bee made honey formulation. In some embodiments, the non-bee made honey comprises one or more proanthocyanidins in an amount from about 15 mg to about 20 mg per 30 mL of the non-bee made honey formulation. In some embodiments, the non-bee made honey comprises one or more proanthocyanidins in an amount from about 0.5 mg to about 20 mg per 30 mL of the non-bee made honey formulation. In some embodiments, the non-bee made honey comprises one or more proanthocyanidins in an amount from 0.0001% to about 0.001% w/w. In some embodiments, the one or more proanthocyanidins are present in an amount from 0.0001% to about 0.001% w/w. In some embodiments, the one or more proanthocyanidins are present in an amount from 0.001% to about 0.01% w/w. In some embodiments, the one or more proanthocyanidins are present in an amount from 0.0001% to about 0.01% w/w. In some embodiments, the one or more proanthocyanidins are present in an amount from 0.0010% to about 0.10% w/w. In some embodiments, the one or more proanthocyanidins are present in an amount from 0.0001% to about 0.10% w/w.

In some embodiments, the one or more polyphenols comprise quercetin in an amount greater than 0.0015% w/w. In some embodiments, the quercetin is present in an amount from about 0.0015% to about 0.10% w/w. In some embodiments, the quercetin is present in an amount of about 0.003% to about 0.005% w/w. In some embodiments, the non-bee made honey formulation comprises one or more polyphenols comprising quercetin in an amount from about 0.005% to about 0.05% w/w. In some embodiments, the non-bee made honey formulation comprises one or more polyphenols comprising quercetin in an amount of about 0.010% w/w.

In some embodiments, the quercetin is present in an amount from about 0.001% to about 0.005% w/w. In some embodiments, the quercetin is present in an amount from about 0.001% to about 0.01% w/w. In some embodiments, the quercetin is present in an amount from about 0.0001% to about 0.005% w/w. In some embodiments, the quercetin is present in an amount from about 0.0001% to about 0.01% w/w. In some embodiments, the quercetin is present in an amount from about 0.000010% to about 0.005% w/w. In some embodiments, the quercetin is present in an amount from about 0.00001% to about 0.01% w/w. In some embodiments, the quercetin is present in an amount from about 0.00010% to about 0.10% w/w. In some embodiments, the quercetin is present in an amount from about 0.001% to about 0.5% w/w. In some embodiments, the non-bee made honey formulation comprises one or more polyphenols comprising isorhamnetin in an amount from about 0.001% to about 0.01% w/w. In some embodiments, the isorhamnetin is present in an amount from about 0.001% to about 0.005% w/w. In some embodiments, the isorhamnetin is present in an amount from about 0.001% to about 0.01% w/w. In some embodiments, the isorhamnetin is present in an amount from about 0.0001% to about 0.005% w/w. In some embodiments, the isorhamnetin is present in an amount from about 0.0001% to about 0.01% w/w. In some embodiments, the isorhamnetin is present in an amount from about 0.00001% to about 0.005% w/w. In some embodiments, the isorhamnetin is present in an amount from about 0.00001% to about 0.01% w/w. In some embodiments, the isorhamnetin is present in an amount from about 0.0001% to about 0.1% w/w. In some embodiments, the isorhamnetin is present in an amount from about 0.001% to about 0.5% w/w. In some embodiments, the non-bee made honey formulation comprises one or more polyphenols comprising kaempferol in an amount from about 0.001% to about 0.01% w/w. In some embodiments, the kaempferol is present in an amount from about 0.001% to about 0.005% w/w. In some embodiments, the kaempferol is present in an amount from about 0.001% to about 0.01% w/w. In some embodiments, the kaempferol is present in an amount from about 0.0001% to about 0.005% w/w. In some embodiments, the kaempferol is present in an amount from about 0.0001% to about 0.01% w/w. In some embodiments, the kaempferol is present in an amount from about 0.00001% to about 0.005% w/w. In some embodiments, the kaempferol is present in an amount from about 0.00001% to about 0.01% w/w. In some embodiments, the kaempferol is present in an amount from about 0.0001% to about 0.1% w/w. In some embodiments, the kaempferol is present in an amount from about 0.001% to about 0.5% w/w. In some embodiments, the non-bee made honey formulation comprises one or more polyphenols comprising chrysin in an amount from about 0.001% to about 0.01% w/w. In some embodiments, the chrysin is present in an amount from about 0.001% to about 0.005% w/w. In some embodiments, the chrysin is present in an amount from about 0.001% to about 0.01% w/w. In some embodiments, the chrysin is present in an amount from about 0.0001% to about 0.005% w/w. In some embodiments, the chrysin is present in an amount from about 0.0001% to about 0.01% w/w. In some embodiments, the chrysin is present in an amount from about 0.00001% to about 0.005% w/w. In some embodiments, the chrysin is present in an amount from about 0.00001% to about 0.01% w/w. In some embodiments, the chrysin is present in an amount from about 0.0001% to about 0.1% w/w. In some embodiments, the chrysin is present in an amount from about 0.001% to about 0.5% w/w. In some embodiments, the non-bee made honey formulation comprises one or more polyphenols comprising apigenin in an amount from about 0.001% to about 0.5% w/w. In some embodiments, the apigenin is present in an amount from about 0.001% to about 0.005% w/w. In some embodiments, the apigenin is present in an amount from about 0.001% to about 0.01% w/w. In some embodiments, the apigenin is present in an amount from about 0.0001% to about 0.005% w/w. In some embodiments, the apigenin is present in an amount from about 0.0001% to about 0.01% w/w. In some embodiments, the apigenin is present in an amount from about 0.00001% to about 0.005% w/w. In some embodiments, the apigenin is present in an amount from about 0.00001% to about 0.01% w/w. In some embodiments, the apigenin is present in an amount from about 0.0001% to about 0.10% w/w. In some embodiments, the apigenin is present in an amount from about 0.001% to about 0.5% w/w. In some embodiments, the non-bee made honey formulation comprises one or more polyphenols comprising apigenin in an amount of about 0.003% w/w. In some embodiments, the apigenin is present in an amount of about 0.01% w/w. In some embodiments, the apigenin is present in an amount of about 0.001% w/w. In some embodiments, the apigenin is present in an amount of about 0.10% w/w.

### Extracts

In some embodiments, the non-bee honey disclosed herein comprises an extract of a plant, fungus, insect, or bacteria. In some embodiments, the non-bee honey disclosed herein comprises an extract of a plant. In some embodiments, the non-bee honey disclosed herein comprises an extract of a fungus. In some embodiments, the non-bee honey disclosed herein comprises an extract of an insect. In some embodiments, the non-bee honey disclosed herein comprises an extract of a bacteria.

Sea Buckthorn Extract—Sea buckthorn (Hippophae rhamnoides) is a plant found throughout Europe and Asia, Sea buckthorn may contain vitamins A, B1, B2, B6, and C, omega-6-fatty acids, various polyphenols, antioxidants, among other compounds. Concentrated sea buckthorn extract may comprise a number of polyphenol compounds, and may contribute the various properties of those polyphenol compounds when used as a component of a non-bee made honey formulation. Sea buckthorn extract may comprise proanthocyanidins and the polyphenols kaempferol, quercetin, and isorhamnetin. In some embodiments, the non-bee made honey comprises sea buckthorn extract. In some embodiments, the non-bee made honey comprises sea buckthorn extract in an amount of from 0.01% to about 1% w/w of the non-bee made honey. In some embodiments, the non-bee made honey comprises sea buckthorn extract in an amount from 0.05% to about 0.5% w/w. In some embodiments, the non-bee made honey comprises sea buckthorn extract in an amount from 0.005% to about 0.5% w/w. In some embodiments, the non-bee made honey comprises sea buckthorn extract in an amount from 0.0005% to about 0.5% w/w. In some embodiments, the non-bee made honey comprises sea buckthorn extract in an amount from 0.00005% to about 0.5% w/w. In some embodiments, the non-bee made honey comprises sea buckthorn extract in an amount of about 0.1% w/w. In some embodiments, the non-bee made honey comprises sea buckthorn extract in an amount of about 0.01% w/w. In some embodiments, the non-bee made honey comprises sea buckthorn extract in an amount of about 0.5% w/w. Sea buckthorn extract can comprise Flavonols such as Quercetin, Kaempferol, Isorhamnetin, Flavones such as, Luteolin Phenolic acids such as, Gallic acid, Ellagic acid, Ferulic acid, Procyanidins, Quinones such as, α-tocopherolquinone, or Ubiquinone. In some cases, the addition of sea buckthorn extract to the formulation may enhance the honey-like qualities of the non-bee made honey formulation.

Jasmine extract—In some embodiments, the plant-based honey comprises jasmine flower extract. In some embodiments, the extract is an aqueous extract of jasmine flower. In some embodiments, the extract is an organic extract of jasmine flower. In some embodiments, the extract is an aerial extract of jasmine flower. In some embodiments, the plant-based honey comprises jasmine extract. In some embodiments, the extract is an extract of jasmine. In some embodiments, the extract is an aqueous extract of jasmine. In some embodiments, the extract is an organic extract of jasmine. In some embodiments, the extract is an aerial extract of jasmine. In some embodiments, the extract is an extract of jasmine roots. In some embodiments, the non-bee made honey comprises a Jasmine extract in an amount of about 0.0001% to about 0.01% w/w. In some embodiments, the non-bee made honey comprises a Jasmine extract in an amount of about 0.001% to about 0.1% w/w. In some embodiments, the non-bee made honey comprises a Jasmine extract in an amount of about 0.000010% to about 0.010% w/w. In some embodiments, the non-bee made honey comprises a Jasmine extract in an amount of about 0.00001% to about 0.001% w/w. Jasmine is an edible and fragrant flower. Jasmine and jasmine extracts may contain phenolic compounds similar to those found in honey such as gallic acid and caffeic acid. Jasmine may comprise catechins and flavan-3-ols such as epicatechin, epicatechin gallate, epigallocatechin, or epigallocatechin gallate (egcg); phenolic acids such as, gallic acid, or caffeic acid; and/or benzyl acetate. In some cases, the addition of jasmine or jasmine extract to the formulation may enhance the honey-like qualities of the non-bee made honey formulation.

Passion Flower—In some embodiments, the plant-based honey comprises Passion flower extract. In some embodiments, the extract is an aqueous extract of Passion flower. In some embodiments, the extract is an organic extract of Passion flower. In some embodiments, the extract is an aerial extract of Passion flower. In some embodiments, the extract is an extract of Passion flower roots. In some embodiments, the non-bee made honey comprises a Passion Flower extract in an amount of about 0.0001% to about 0.01% w/w. In some embodiments, the non-bee made honey comprises a Passion Flower extract in an amount of about 0.001% to about 0.10% w/w. In some embodiments, the non-bee made honey comprises a Passion Flower extract in an amount of about 0.00001% to about 0.01% w/w. In some embodiments, the non-bee made honey comprises a Passion Flower extract in an amount of about 0.00001% to about 0.001% w/w. Passion Flower is an edible flower that may contain phenolic compounds similar to those found in bee-made honey, such as apigenin, luteolin, quercetin, kaempferol, and chrysin, as well as chlorogenic acid, caffeic acid, and ferulic acid. Passion flower and passion flower extracts may contain flavonoids such as apigenin luteolin, quercetin, kaempferol and/or isovitexin chrysin; c-glycosylflavones such as orientin, vitexin, isovitexin, and/or isoorientin; coumarins such as scopoletin, umbelliferone; phenolic acids such as chlorogenic acid, caffeic acid and/or ferulic acid. In some cases, the addition of Passion flower or passion flower extract to the formulation may enhance the honey-like qualities of the non-bee made honey formulation.

Red Clover—In some embodiments, the plant-based honey comprises Red Clover extract. In some embodiments, the extract is an aqueous extract of Red Clover. In some embodiments, the extract is an organic extract of Red Clover. In some embodiments, the extract is an aerial extract of Red Clover. In some embodiments, the extract is an extract of Red Clover roots. In some embodiments, the non-bee made honey comprises a Red Clover extract in an amount of about 0.0001% to about 0.01% w/w. In some embodiments, the non-bee made honey comprises a Red Clover extract in an amount of about 0.001% to about 0.1% w/w. In some embodiments, the non-bee made honey comprises a Red Clover extract in an amount of about 0.00001% to about 0.01% w/w. In some embodiments, the non-bee made honey comprises a Red Clover extract in an amount of about 0.00001% to about 0.001% w/w. Red Clover is an edible flower that may contain phenolic compounds similar to those found in bee-made honey such as caffeic acid, ferulic acid, quercetin, apigenin and kaempferol. Red Clover or Red Clover extracts may contain isoflavones such as formononeith biochanin A daidzein and /orgenistein; coumarins such as coumestrol, medicagol; phenolic acids such as caffeic acid, p-coumaric acid, ferulic acid, and/or p-hydroxybenzoic acid; flavonoids such as quercetin, kaempferol, and/or apigenin. In some cases, the addition of Red Clover or Red Clover extract to the formulation may enhance the honey-like qualities of the non-bee made honey formulation.

Lavender—In some embodiments, the plant-based honey comprises Lavender extract. In some embodiments, the extract is an aqueous extract of Lavender. In some embodiments, the extract is an organic extract of Lavender. In some embodiments, the extract is an aerial extract of Lavender. In some embodiments, the extract is an extract of Lavender roots. In some embodiments, the non-bee made honey comprises a Lavender extract in an amount of about 0.0001% to about 0.01% w/w. In some embodiments, the non-bee made honey comprises a Lavender extract in an amount of about 0.001% to about 0.1% w/w. In some embodiments, the non-bee made honey comprises a Lavender extract in an amount of about 0.00001% to about 0.01% w/w. In some embodiments, the non-bee made honey comprises a Lavender extract in an amount of about 0.00001% to about 0.001% w/w. Lavender is an edible and fragrant flower. Lavender or lavender extracts may contain phenolic compounds similar to those found in honey such as caffeic acid and flavonoids like luteolin or apigenin. Lavender or lavender extracts may contain phenolic acids such as caffeic acid derivatives, rosmarinic acid, salvianolic acid; flavonoids such as luteolin, apigenin; coumarins such as herniar, umbelliferone; triterpenes such as ursolic acid, oleanolic acid. In some cases, the addition of Lavender or lavender extract to the formulation may enhance the honey-like qualities of the non-bee made honey formulation.

Chamomile—In some embodiments, the plant-based honey comprises Chamomile extract. In some embodiments, the extract is an aqueous extract of Chamomile. In some embodiments, the extract is an organic extract of Chamomile. In some embodiments, the extract is an aerial extract of Chamomile. In some embodiments, the extract is an extract of Chamomile roots. In some embodiments, the non-bee made honey comprises a Chamomile extract in an amount of about 0.0001% to about 0.01% w/w. In some embodiments, the non-bee made honey comprises a Chamomile extract in an amount of about 0.001% to about 0.1% w/w. In some embodiments, the non-bee made honey comprises a Chamomile extract in an amount of about 0.00001% to about 0.01% w/w. In some embodiments, the non-bee made honey comprises a Chamomile extract in an amount of about 0.00001% to about 0.001% w/w. Chamomile is an edible and fragrant flower. Chamomile and chamomile extracts may contain phenolic compounds similar to those found in honey such as chlorogenic acid, caffeic acid, and flavonoids like apigenin, quercetin, and luteolin. Chamomile and chamomile extracts may contain flavonoids such as, apigenin, quercetin, luteolin, coumarins such as, hemiarin, umbelliferone, phenolic acids such as, caffeic acids, chlorogenic acid, ferulic acid, sesquiterpenes such as, α-bisabolol, chamazuelene. In some cases, the addition of Chamomile and chamomile extracts to the formulation may enhance the honey-like qualities of the non-bee made honey formulation.

White tea—In some embodiments, the plant-based honey comprises White tea extract. In some embodiments, the extract is an aqueous extract of White tea. In some embodiments, the extract is an organic extract of White tea. In some embodiments, the extract is an aerial extract of White tea. In some embodiments, the extract is an extract of White tea roots. White Tea can contain phenolic compounds similar to those found in honey, such as flavonols like quercetin, kaempferol, and myricetin, and phenolic acids like gallic acid, caffeic acid, and chlorogenic acid. White Tea and White Tea extracts can contain Catechins: similar to the ones found in green tea such as Epicatechin gallate, Epigallocatechin, Epigallocatechin gallate (EGCG), Flavonols such as, Quercetin, Kaempferol, Myricetin, Phenolic Acids such as, Gallic acid, Caffeic Acid, Chlorogenic acid, and Theaflavins and Thearubigins. In some cases, the addition of White Tea and White Tea extracts to the formulation may enhance the honey-like qualities of the non-bee made honey formulation.

Grape Seed—In some embodiments, the plant-based honey comprises Grape seed extract. In some embodiments, the extract is an aqueous extract of Grape seed. In some embodiments, the extract is an organic extract of Grape seed. Grape Seed may contain phenolic compounds similar to those found in bee-made honey, such as flavonoids like quercetin, kaempferol, and myricetin, and phenolic acids like gallic acid and caffeic acid. Grape seed extract can contain Proanthocyanidins

Flavonoids such as, Quercetin, Kaempferol, Myricetin, Phenolic acids such as, Gallic acid, Caffeic acid, Protocatechuic acid, Stilbenes such as, Resveratol. In some cases, the addition of Grape seed extract to the formulation may enhance the honey-like qualities of the non-bee made honey formulation.

In some embodiments, the plant-based honey comprises one or more extracts of natural compositions. In some embodiments, the plant-based honey comprises one or more extracts comprising polyphenols. In some embodiments, the plant-based honey comprises sea buckthorn extract, and one or more extracts selected from passion flower, red clover, chamomile, lavender, or jasmine; and the selection and combination of extracts provides for a high antioxidant and/or high phenolic content to the plant-based honey formulation, while also providing for a sweet honey like taste that is not disturbed or altered by the high antioxidant and/or high phenolic content. In some cases, the sweet honey like taste closely approximates the mild floral notes of bee made clover honey. In some embodiments, the plant-based honey has an appendence, color, and cohesive quality which is similar to that of bee made honey.

In some embodiments, the plant-based honey comprises one or more extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder), or a combination thereof. In some embodiments, the plant-based extract comprises two extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises three extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises four extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises five extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises six extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises Sea Buckthorn extract and one or more extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises Sea Buckthorn extract and two or more extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises Sea Buckthorn extract and three extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises Sea Buckthorn extract and four extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises Sea Buckthorn extract and five extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder). In some embodiments, the plant-based extract comprises Sea Buckthorn extract and six extracts selected from: Jasmine extract, passion flower extract, red clover extract, lavender extract, white tea extract, green tea extract, red tea extract, grape seed extract, sea buckthorn extract, Reishi mushroom powder, Chaga mushroom powder, Green coffee bean powder, Ginger powder, Pu'erh tea powder, Matcha powder, Acai berry powder, Aronia berry powder, Hibiscus flower, Squash blossom extract, Chamomile flower extract, Cinnamon stick extract, Orange blossom extract, Lemon or lemon juice extract, Shilajit (mineral powder).

Antioxidant activity of compositions disclosed herein may vary with specific formulations of honey and can be assessed with methods known in the art, such as with a Sigma MAK187 Trolox equivalency assay. In some embodiments, the total antioxidant capacity is measured by a Trolox equivalent assay, and expressed as Trolox equivalents in nmol/μL. In some embodiments, the non-bee made honey has an antioxidant activity of at least 10 nmol/μL. In some embodiments, the plant based honey has an antioxidant activity of at least 10 nmol/μL. In some embodiments, the non-bee made honey has an antioxidant activity of at least 100 nmol/μL. In some embodiments, the plant based honey has an antioxidant activity of at least 100 nmol/μL. In some embodiments, the non-bee made honey has an antioxidant activity of at least 1,000 nmol/μL. In some embodiments, the plant based honey has an antioxidant activity of at least 1,000 nmol/μL. In some embodiments, the plant based honey has an antioxidant activity of at least 100 nmol/μL. In some embodiments, the non-bee made honey has an antioxidant activity of at least 10 mmol/μL. In some embodiments, the plant based honey has an antioxidant activity of at least 10 mmol/μL. In some embodiments, the non-bee made honey has an antioxidant activity of at least 100 mmol/μL. In some embodiments, the plant based honey has an antioxidant activity of at least 100 mmol/μL. In some embodiments, the non-bee made honey has an antioxidant activity of between about 10 nmol/μL and about 1 mmol/μL. In some embodiments, the plant-based honey has an antioxidant activity of between about 10 nmol/μL and about 1 mmol/μL. In some embodiments, the non-bee made honey has an antioxidant activity of between about 100 nmol/μL and about 1 mmol/μL. In some embodiments, the plant-based honey has an antioxidant activity of between about 100 nmol/μL and about 1 mmol/μL. In some embodiments, the non-bee made honey has an antioxidant activity of between about 10 nmol/μL and about 1 mmol/μL. In some embodiments, the plant-based honey has an antioxidant activity of between about 10 nmol/μL and about 1 mmol/μL. In some embodiments, the non-bee made honey has an antioxidant activity of between about 10 nmol/μL and about 500 nmol/μL. In some embodiments, the plant-based honey has an antioxidant activity of between about 10 nmol/μL and about 500 nmol/μL. In some embodiments, the non-bee made honey has an antioxidant activity of between about 1 nmol/μL and about 500 nmol/μL. In some embodiments, the plant-based honey has an antioxidant activity of between about 1 nmol/μL and about 500 nmol/μL. In some embodiments, the total antioxidant capacity is at least that of natural bee-made clover honey. In some embodiments, the total antioxidant capacity exceeds that of natural bee-made clover honey. In some embodiments, the total antioxidant capacity exceeds that of natural bee-made clover honey by at least 2.5% wt. In some embodiments, the total antioxidant capacity is at least 2,500 nM/uL Trolox Equivalents. In some embodiments, high antioxidant capacity may be due to one or more extracts comprised within the formulation selected from passion flower, red clover, chamomile, lavender, or jasmine; and the selection and combination of extracts provides for a high antioxidant to the plant-based honey formulation, while also providing for a sweet honey like taste that is not disturbed or altered by the high antioxidant. In some cases, the sweet honey like taste closely approximates the mild floral notes of bee made clover honey, and the plant-based honey has an appendence, color, and cohesive quality which is similar to that of bee made honey notwithstanding the high antioxidant activity.

The non-bee made and plant-based honey compositions may demonstrate a variety of water activity values. In some embodiments, the non-bee made honey composition has a water activity of about 0.4. In some embodiments, the plant based honey composition has a water activity of about 0.4. In some embodiments, the non-bee made honey composition has a water activity of about 0.5. In some embodiments, the plant based honey composition has a water activity of about 0.5. In some embodiments, the non-bee made honey composition has a water activity of about 0.45. In some embodiments, the plant based honey composition has a water activity of about 0.45. In some embodiments, the non-bee made honey composition has a water activity of between about 0.4 and 0.5. In some embodiments, the plant based honey composition has a water activity of between about 0.4 and 0.5. In some embodiments, the non-bee made honey composition has a water activity of between about 0.35 and 0.5. In some embodiments, the plant based honey composition has a water activity of between about 0.35 and 0.5. In some embodiments, the non-bee made honey composition has a water activity of between about 0.35 and 0.55. In some embodiments, the plant based honey composition has a water activity of between about 0.35 and 0.55.

In the plant-based honey described herein, special attention has been placed on the inclusion of polyphenols in the composition, both for their flavor, pigment, and most importantly for their health benefits. The preferred compounds may be substituted or enhanced with other polyphenolic compounds, all of which are found in flowering plants, that eventually may become a component of bee-made honey. Importantly, in order to increase bioavailability to the consumer, as well as enhance the natural anti-bacterial properties of the polyphenolic compounds included in the described plant-based honey formulation, these compounds may be included in an order of magnitude between 5 to 1000 times that found in bee-made honey.

Several examples of possible ingredients as well as extracts are shown in table 1.

TABLE 1
Ingredients by type for use in non-bee made honey.

| Ingredient | Type |
|---|---|
| Fructose | Sugar |
| Glucose (dextrose) monohydrate | Sugar |
| Glucose (dextrose) anhydrous | Sugar |
| Sucrose | Sugar |
| Maltose | Sugar |
| Malto-dextrin | Sugar |
| Coconut sugar | Sugar |
| Brown sugar | Sugar |
| Gluconic acid | Acid |
| Succinic acid | Acid |
| Gallic acid | Acid |
| Chlorogenic acid | Acid |
| Ferulic acid | Acid |
| Cinnamic acid | Acid |
| Kojic acid | Acid |
| Caffeic acid | Acid |
| Glucono-delta-lactone | Acid |
| Citric acid | Acid |
| Lactic acid | Acid |
| p-coumaric acid | Acid |
| Malic acid | Acid |
| Tartaric acid | Acid |
| Reishi mushroom powder | Polyphenol |
| Chaga mushroom powder | Plant ingredient |
| Green coffee bean powder | Plant ingredient |
| Ginger powder | Plant ingredient |
| Pu'erh tea powder | Plant ingredient |
| Matcha powder | Plant ingredient |
| Acai berry powder | Plant ingredient |
| Aronia berry powder | Plant ingredient |
| Hibiscus flower | Plant ingredient |
| Squash blossom | Plant ingredient |
| Chamomile flower | Plant ingredient |
| Cinnamon stick | Plant ingredient |
| Orange blossom | Plant ingredient |
| Green tea | Plant ingredient |
| Lemon juice | Plant ingredient |
| Shilajit (mineral powder) | Plant ingredient |
| Chamomile | Plant ingredient |
| Red Clover | Plant ingredient |
| Jasmine | Plant ingredient |
| Lavender | Plant ingredient |
| Passionflower | Plant ingredient |
| Apigenin | Flavanoid/Polyphenol |
| Chrysin | Flavanoid/Polyphenol |
| Kaempferol | Flavanoid/Polyphenol |
| Luteolin | Flavanoid/Polyphenol |
| Naringenin | Flavanoid/Polyphenol |
| Quercetin | Flavanoid/Polyphenol |
| Rutin | Flavanoid/Polyphenol |
| Pinocembrin | Flavanoid/Polyphenol |
| Protocatechualdehyde | Flavanoid/Polyphenol |

In some embodiments, the non-bee made honey formulation comprises one or more extracts are selected from: Sea buckthorn, Chamomile, Red Clover, Jasmine, Lavender, Succinic acid, or Passionflower. In some embodiments, the non-bee made honey formulation comprises Sea buckthorn extract, and one or more extracts selected from: Chamomile, Red Clover, Jasmine, Lavender, Succinic acid, or Passionflower.

In some embodiments, the non-bee made honey formulation comprises Sea buckthorn extract, and three or more extracts selected from: Chamomile, Red Clover, Jasmine, Lavender, Succinic acid, or Passionflower. The non-bee made honey composition of claim 1, further comprising Sea buckthorn extract, Chamomile extract, Red Clover, and Succinic acid. In some embodiments, the non-bee made honey formulation comprises one or more extracts comprising: Sea buckthorn, Reishi mushroom, Chaga mushroom, Green coffee bean, Ginger, Pu'erh tea, Matcha, Acai berry, Aronia berry, Hibiscus, Squash blossom, Chamomile, Cinnamon, Orange blossom, Green tea, Lemon or lemon juice, white tea, grape seed, or Shilajit (mineral powder). In some embodiments, the non-bee made honey formulation comprises at least two extracts selected from: Sea buckthorn, Reishi mushroom, Chaga mushroom, Green coffee bean, Ginger, Pu'erh tea, Matcha, Acai berry, Aronia berry, Hibiscus, Squash blossom, Chamomile, Cinnamon, Orange blossom, Green tea, Lemon, white tea, grape seed, or Shilajit (mineral powder). In some embodiments, the non-bee made honey formulation comprises at least three extracts that are selected from: Reishi mushroom, Chaga mushroom, Green coffee bean powder, Ginger powder, Pu'erh tea, Matcha, Acai berry, Aronia berry, Hibiscus flower, Squash blossom, Chamomile flower, Cinnamon, Orange blossom, Green tea, Lemon, or Shilajit.

In some embodiments, the non-bee made honey formulation comprises Sea buckthorn extract and at least two extracts selected from: Reishi mushroom, Chaga mushroom, Green coffee bean, Ginger, Pu'erh tea, Matcha, Acai berry, Aronia berry, Hibiscus flower, Squash blossom, Chamomile flower, Cinnamon stick, Orange blossom, Green tea, Lemon or lemon juice, or Shilajit (mineral powder). In some embodiments, the non-bee made honey formulation comprises Sea buckthorn extract and at least three extracts selected from: Reishi mushroom powder, Chaga mushroom, Green coffee bean, Ginger, Pu'erh tea, Matcha, Acai berry, Aronia berry, Hibiscus flower, Squash blossom, Chamomile, Cinnamon stick, Orange blossom, Green tea, Lemon, or Shilajit (mineral powder).

In some embodiments, the non-bee made honey comprises at least one ingredient shown in Table 5 wherein the at least one ingredient is optionally in the stated percentage of Table 5. In some embodiments, the non-bee made honey comprises ingredients shown in Table 6 wherein the at least one ingredient is optionally in the stated percentage of Table 6. In some embodiments, the non-bee made honey comprises ingredients shown in Table 7, wherein the at least one ingredient is optionally in the stated percentage of Table 7. In some embodiments, the non-bee made honey comprises ingredients shown in Table 8, wherein the at least one ingredient is optionally in the stated percentage of Table 8.

Several of the ingredients are polyphenols themselves such as quercetin and kaempferol, and others are extracts of plants rich in polyphenols such as Sea Buckthorn and Green tea. By adding the distinct extracts to honey formulations, a new profile of sweetener can be created rapidly, whereas adding and removing single polyphenol constituents provides greater control over the qualities of the resulting substitute. By determining the properties of each extract, and determining the resulting appeal of the honey with the properties of the polyphenols as they may be contributed to the formulation, valuable polyphenol profiles of non-bee made honey formulations can be determined.

In some embodiments, the non-bee made honey composition comprises a plant extract in an amount between about 0.001% w/w to about 3% w/w. In some embodiments, the non-bee made honey composition comprises a plant extract in an amount between about 0.001% w/w to about 0.005% w/w, about 0.001% w/w to about 0.01% w/w, about 0.001% w/w to about 0.05% w/w, about 0.001% w/w to about 0.1% w/w, about 0.001% w/w to about 0.4% w/w, about 0.001% w/w to about 0.8% w/w, about 0.001% w/w to about 1% w/w, about 0.001% w/w to about 1.5% w/w, about 0.001% w/w to about 2% w/w, about 0.001% w/w to about 2.5% w/w, about 0.001% w/w to about 3% w/w, about 0.005% w/w to about 0.01% w/w, about 0.005% w/w to about 0.05% w/w, about 0.005% w/w to about 0.1% w/w, about 0.005% w/w to about 0.4% w/w, about 0.005% w/w to about 0.8% w/w, about 0.005% w/w to about 1% w/w, about 0.005% w/w to about 1.5% w/w, about 0.005% w/w to about 2% w/w, about 0.005% w/w to about 2.5% w/w, about 0.005% w/w to about 3% w/w, about 0.01% w/w to about 0.05% w/w, about 0.01% w/w to about 0.1% w/w, about 0.01% w/w to about 0.4% w/w, about 0.01% w/w to about 0.8% w/w, about 0.01% w/w to about 1% w/w, about 0.01% w/w to about 1.5% w/w, about 0.01% w/w to about 2% w/w, about 0.01% w/w to about 2.5% w/w, about 0.01% w/w to about 3% w/w, about 0.05% w/w to about 0.1% w/w, about 0.05% w/w to about 0.4% w/w, about 0.05% w/w to about 0.8% w/w, about 0.05% w/w to about 1% w/w, about 0.05% w/w to about 1.5% w/w, about 0.05% w/w to about 2% w/w, about 0.05% w/w to about 2.5% w/w, about 0.05% w/w to about 3% w/w, about 0.1% w/w to about 0.4% w/w, about 0.1% w/w to about 0.8% w/w, about 0.1% w/w to about 1% w/w, about 0.1% w/w to about 1.5% w/w, about 0.1% w/w to about 2% w/w, about 0.1% w/w to about 2.5% w/w, about 0.1% w/w to about 3% w/w, about 0.4% w/w to about 0.8% w/w, about 0.4% w/w to about 1% w/w, about 0.4% w/w to about 1.5% w/w, about 0.4% w/w to about 2% w/w, about 0.4% w/w to about 2.5% w/w, about 0.4% w/w to about 3% w/w, about 0.8% w/w to about 1% w/w, about 0.8% w/w to about 1.5% w/w, about 0.8% w/w to about 2% w/w, about 0.8% w/w to about 2.5% w/w, about 0.8% w/w to about 3% w/w, about 1% w/w to about 1.5% w/w, about 1% w/w to about 2% w/w, about 1% w/w to about 2.5% w/w, about 1% w/w to about 3% w/w, about 1.5% w/w to about 2% w/w, about 1.5% w/w to about 2.5% w/w, about 1.5% w/w to about 3% w/w, about 2% w/w to about 2.5% w/w, about 2% w/w to about 3% w/w, or about 2.5% w/w to about 3% w/w, including increments therein. In some embodiments, the non-bee made honey composition comprises a plant extract in an amount between about 0.001% w/w, about 0.005% w/w, about 0.01% w/w, about 0.05% w/w, about 0.1% w/w, about 0.4% w/w, about 0.8% w/w, about 1% w/w, about 1.5% w/w, about 2% w/w, about 2.5% w/w, or about 3% w/w. In some embodiments, the non-bee made honey composition comprises a plant extract in an amount between at least about 0.001% w/w, about 0.005% w/w, about 0.01% w/w, about 0.05% w/w, about 0.1% w/w, about 0.4% w/w, about 0.8% w/w, about 1% w/w, about 1.5% w/w, about 2% w/w, or about 2.5% w/w. In some embodiments, the non-bee made honey composition comprises a plant extract in an amount between at most about 0.005% w/w, about 0.01% w/w, about 0.05% w/w, about 0.1% w/w, about 0.4% w/w, about 0.8% w/w, about 1% w/w, about 1.5% w/w, about 2% w/w, about 2.5% w/w, or about 3% w/w. In some embodiments, a plant extract comprises between 3 and 0.001% w/w of the non-bee made honey composition. In some embodiments, a plant extract comprises between 0.5 and 0.05% w/w of the non-bee made honey composition. In some embodiments, a plant extract comprises between 3 and 0.1% w/w of the non-bee made honey composition. In some embodiments, a plant extract comprises between 0.1 and 0.05% w/w of the non-bee made honey composition. In some embodiments, a plant extract comprises between 0.01 and 0.001% w/w of the non-bee made honey composition. In some embodiments, the plant-based honey composition comprises a plant extract in an amount between about 0.001% w/w to about 3% w/w. In some embodiments, the plant-based honey composition comprises a plant extract in an amount between about 0.001% w/w to about 0.005% w/w, about 0.001% w/w to about 0.01% w/w, about 0.001% w/w to about 0.05% w/w, about 0.001% w/w to about 0.1% w/w, about 0.001% w/w to about 0.4% w/w, about 0.001% w/w to about 0.8% w/w, about 0.001% w/w to about 1% w/w, about 0.001% w/w to about 1.5% w/w, about 0.001% w/w to about 2% w/w, about 0.001% w/w to about 2.5% w/w, about 0.001% w/w to about 3% w/w, about 0.005% w/w to about 0.01% w/w, about 0.005% w/w to about 0.05% w/w, about 0.005% w/w to about 0.1% w/w, about 0.005% w/w to about 0.4% w/w, about 0.005% w/w to about 0.8% w/w, about 0.005% w/w to about 1% w/w, about 0.005% w/w to about 1.5% w/w, about 0.005% w/w to about 2% w/w, about 0.005% w/w to about 2.5% w/w, about 0.005% w/w to about 3% w/w, about 0.01% w/w to about 0.05% w/w, about 0.01% w/w to about 0.1% w/w, about 0.01% w/w to about 0.4% w/w, about 0.01% w/w to about 0.8% w/w, about 0.01% w/w to about 1% w/w, about 0.01% w/w to about 1.5% w/w, about 0.01% w/w to about 2% w/w, about 0.01% w/w to about 2.5% w/w, about 0.01% w/w to about 3% w/w, about 0.05% w/w to about 0.1% w/w, about 0.05% w/w to about 0.4% w/w, about 0.05% w/w to about 0.8% w/w, about 0.05% w/w to about 1% w/w, about 0.05% w/w to about 1.5% w/w, about 0.05% w/w to about 2% w/w, about 0.05% w/w to about 2.5% w/w, about 0.05% w/w to about 3% w/w, about 0.1% w/w to about 0.4% w/w, about 0.1% w/w to about 0.8% w/w, about 0.1% w/w to about 1% w/w, about 0.1% w/w to about 1.5% w/w, about 0.1% w/w to about 2% w/w, about 0.1% w/w to about 2.5% w/w, about 0.1% w/w to about 3% w/w, about 0.4% w/w to about 0.8% w/w, about 0.4% w/w to about 1% w/w, about 0.4% w/w to about 1.5% w/w, about 0.4% w/w to about 2% w/w, about 0.4% w/w to about 2.5% w/w, about 0.4% w/w to about 3% w/w, about 0.8% w/w to about 1% w/w, about 0.8% w/w to about 1.5% w/w, about 0.8% w/w to about 2% w/w, about 0.8% w/w to about 2.5% w/w, about 0.8% w/w to about 3% w/w, about 1% w/w to about 1.5% w/w, about 1% w/w to about 2% w/w, about 1% w/w to about 2.5% w/w, about 1% w/w to about 3% w/w, about 1.5% w/w to about 2% w/w, about 1.5% w/w to about 2.5% w/w, about 1.5% w/w to about 3% w/w, about 2% w/w to about 2.5% w/w, about 2% w/w to about 3% w/w, or about 2.5% w/w to about 3% w/w, including increments therein. In some embodiments, the plant-based honey composition comprises a plant extract in an amount between about 0.001% w/w, about 0.005% w/w, about 0.01% w/w, about 0.05% w/w, about 0.1% w/w, about 0.4% w/w, about 0.8% w/w, about 1% w/w, about 1.5% w/w, about 2% w/w, about 2.5% w/w, or about 3% w/w. In some embodiments, the plant-based honey composition comprises a plant extract in an amount between at least about 0.001% w/w, about 0.005% w/w, about 0.01% w/w, about 0.05% w/w, about 0.1% w/w, about 0.4% w/w, about 0.8% w/w, about 1% w/w, about 1.5% w/w, about 2% w/w, or about 2.5% w/w. In some embodiments, the plant-based honey composition comprises a plant extract in an amount between at most about 0.005% w/w, about 0.01% w/w, about 0.05% w/w, about 0.1% w/w, about 0.4% w/w, about 0.8% w/w, about 1% w/w, about 1.5% w/w, about 2% w/w, about 2.5% w/w, or about 3% w/w. In some embodiments, a plant extract comprises between 3 and 0.001% w/w of the plant-based honey composition. In some embodiments, a plant extract comprises between 0.5 and 0.05% w/w of the plant-based honey composition. In some embodiments, a plant extract comprises between 3 and 0.1% w/w of the plant-based honey composition. In some embodiments, a plant extract comprises between 0.1 and 0.05% w/w of the plant-based honey composition. In some embodiments, a plant extract comprises between 0.01 and 0.001% w/w of the plant-based honey composition

In some embodiments, the non-bee made honey composition comprises sea buckthorn extract in an amount between about 0.0001% w/w to about 1% w/w. In some embodiments, the non-bee made honey composition comprises sea buckthorn extract in an amount between about 0.0001% w/w to about 0.0005% w/w, about 0.0001% w/w to about 0.001% w/w, about 0.0001% w/w to about 0.0015% w/w, about 0.0001% w/w to about 0.002% w/w, about 0.0001% w/w to about 0.005% w/w, about 0.0001% w/w to about 0.01% w/w, about 0.0001% w/w to about 0.05% w/w, about 0.0001% w/w to about 0.1% w/w, about 0.0001% w/w to about 0.4% w/w, about 0.0001% w/w to about 0.8% w/w, about 0.0001% w/w to about 1% w/w, about 0.0005% w/w to about 0.001% w/w, about 0.0005% w/w to about 0.0015% w/w, about 0.0005% w/w to about 0.002% w/w, about 0.0005% w/w to about 0.005% w/w, about 0.0005% w/w to about 0.01% w/w, about 0.0005% w/w to about 0.05% w/w, about 0.0005% w/w to about 0.1% w/w, about 0.0005% w/w to about 0.4% w/w, about 0.0005% w/w to about 0.8% w/w, about 0.0005% w/w to about 1% w/w, about 0.001% w/w to about 0.0015% w/w, about 0.001% w/w to about 0.002% w/w, about 0.001% w/w to about 0.005% w/w, about 0.001% w/w to about 0.01% w/w, about 0.001% w/w to about 0.05% w/w, about 0.001% w/w to about 0.1% w/w, about 0.001% w/w to about 0.4% w/w, about 0.001% w/w to about 0.8% w/w, about 0.001% w/w to about 1% w/w, about 0.0015% w/w to about 0.002% w/w, about 0.0015% w/w to about 0.005% w/w, about 0.0015% w/w to about 0.01% w/w, about 0.0015% w/w to about 0.05% w/w, about 0.0015% w/w to about 0.1% w/w, about 0.0015% w/w to about 0.4% w/w, about 0.0015% w/w to about 0.8% w/w, about 0.0015% w/w to about 1% w/w, about 0.002% w/w to about 0.005% w/w, about 0.002% w/w to about 0.01% w/w, about 0.002% w/w to about 0.05% w/w, about 0.002% w/w to about 0.1% w/w, about 0.002% w/w to about 0.4% w/w, about 0.002% w/w to about 0.8% w/w, about 0.002% w/w to about 1% w/w, about 0.005% w/w to about 0.01% w/w, about 0.005% w/w to about 0.05% w/w, about 0.005% w/w to about 0.1% w/w, about 0.005% w/w to about 0.4% w/w, about 0.005% w/w to about 0.8% w/w, about 0.005% w/w to about 1% w/w, about 0.01% w/w to about 0.05% w/w, about 0.01% w/w to about 0.1% w/w, about 0.01% w/w to about 0.4% w/w, about 0.01% w/w to about 0.8% w/w, about 0.01% w/w to about 1% w/w, about 0.05% w/w to about 0.1% w/w, about 0.05% w/w to about 0.4% w/w, about 0.05% w/w to about 0.8% w/w, about 0.05% w/w to about 1% w/w, about 0.1% w/w to about 0.4% w/w, about 0.1% w/w to about 0.8% w/w, about 0.1% w/w to about 1% w/w, about 0.4% w/w to about 0.8% w/w, about 0.4% w/w to about 1% w/w, or about 0.8% w/w to about 1% w/w, including increments therein. In some embodiments, the non-bee made honey composition comprises sea buckthorn extract in an amount between about 0.0001% w/w, about 0.0005% w/w, about 0.001% w/w, about 0.0015% w/w, about 0.002% w/w, about 0.005% w/w, about 0.01% w/w, about 0.05% w/w, about 0.1% w/w, about 0.4% w/w, about 0.8% w/w, or about 1% w/w. In some embodiments, the non-bee made honey composition comprises sea buckthorn extract in an amount between at least about 0.0001% w/w, about 0.0005% w/w, about 0.001% w/w, about 0.0015% w/w, about 0.002% w/w, about 0.005% w/w, about 0.01% w/w, about 0.05% w/w, about 0.1% w/w, about 0.4% w/w, or about 0.8% w/w. In some embodiments, the non-bee made honey composition comprises sea buckthorn extract in an amount between at most about 0.0005% w/w, about 0.001% w/w, about 0.0015% w/w, about 0.002% w/w, about 0.005% w/w, about 0.01% w/w, about 0.05% w/w, about 0.1% w/w, about 0.4% w/w, about 0.8% w/w, or about 1% w/w. In some embodiments, the non-bee made honey formulation may comprise sea buckthorn extract in an amount of from 0.001% to about 1% w/w of the formulation. In some embodiments, the non-bee made honey formulation may comprise sea buckthorn extract in an amount of from 0.01% to about 1% w/w of the formulation. In some embodiments, the non-bee made honey formulation may comprise sea buckthorn extract in an amount of from 0.1% to about 1% w/w of the formulation. In some embodiments, the non-bee made honey formulation may comprise sea buckthorn extract in an amount from 0.05% to about 0.5% w/w. The non-bee made honey formulation may comprise sea buckthorn extract in an amount of about 0.1% w/w. In some embodiments, the plant-based honey composition comprises sea buckthorn extract in an amount between about 0.0001% w/w to about 1% w/w. In some embodiments, the plant-based honey composition comprises sea buckthorn extract in an amount between about 0.0001% w/w to about 0.0005% w/w, about 0.0001% w/w to about 0.001% w/w, about 0.0001% w/w to about 0.0015% w/w, about 0.0001% w/w to about 0.002% w/w, about 0.0001% w/w to about 0.005% w/w, about 0.0001% w/w to about 0.01% w/w, about 0.0001% w/w to about 0.05% w/w, about 0.0001% w/w to about 0.1% w/w, about 0.0001% w/w to about 0.4% w/w, about 0.0001% w/w to about 0.8% w/w, about 0.0001% w/w to about 1% w/w, about 0.0005% w/w to about 0.001% w/w, about 0.0005% w/w to about 0.0015% w/w, about 0.0005% w/w to about 0.002% w/w, about 0.0005% w/w to about 0.005% w/w, about 0.0005% w/w to about 0.01% w/w, about 0.0005% w/w to about 0.05% w/w, about 0.0005% w/w to about 0.1% w/w, about 0.0005% w/w to about 0.4% w/w, about 0.0005% w/w to about 0.8% w/w, about 0.0005% w/w to about 1% w/w, about 0.001% w/w to about 0.0015% w/w, about 0.001% w/w to about 0.002% w/w, about 0.001% w/w to about 0.005% w/w, about 0.001% w/w to about 0.01% w/w, about 0.001% w/w to about 0.05% w/w, about 0.001% w/w to about 0.1% w/w, about 0.001% w/w to about 0.4% w/w, about 0.001% w/w to about 0.8% w/w, about 0.001% w/w to about 1% w/w, about 0.0015% w/w to about 0.002% w/w, about 0.0015% w/w to about 0.005% w/w, about 0.0015% w/w to about 0.01% w/w, about 0.0015% w/w to about 0.05% w/w, about 0.0015% w/w to about 0.1% w/w, about 0.0015% w/w to about 0.4% w/w, about 0.0015% w/w to about 0.8% w/w, about 0.0015% w/w to about 1% w/w, about 0.002% w/w to about 0.005% w/w, about 0.002% w/w to about 0.01% w/w, about 0.002% w/w to about 0.05% w/w, about 0.002% w/w to about 0.1% w/w, about 0.002% w/w to about 0.4% w/w, about 0.002% w/w to about 0.8% w/w, about 0.002% w/w to about 1% w/w, about 0.005% w/w to about 0.01% w/w, about 0.005% w/w to about 0.05% w/w, about 0.005% w/w to about 0.1% w/w, about 0.005% w/w to about 0.4% w/w, about 0.005% w/w to about 0.8% w/w, about 0.005% w/w to about 1% w/w, about 0.01% w/w to about 0.05% w/w, about 0.01% w/w to about 0.1% w/w, about 0.01% w/w to about 0.4% w/w, about 0.01% w/w to about 0.8% w/w, about 0.01% w/w to about 1% w/w, about 0.05% w/w to about 0.1% w/w, about 0.05% w/w to about 0.4% w/w, about 0.05% w/w to about 0.8% w/w, about 0.05% w/w to about 1% w/w, about 0.1% w/w to about 0.4% w/w, about 0.1% w/w to about 0.8% w/w, about 0.1% w/w to about 1% w/w, about 0.4% w/w to about 0.8% w/w, about 0.4% w/w to about 1% w/w, or about 0.8% w/w to about 1% w/w, including increments therein. In some embodiments, the plant-based honey composition comprises sea buckthorn extract in an amount between about 0.0001% w/w, about 0.0005% w/w, about 0.001% w/w, about 0.0015% w/w, about 0.002% w/w, about 0.005% w/w, about 0.01% w/w, about 0.05% w/w, about 0.1% w/w, about 0.4% w/w, about 0.8% w/w, or about 1% w/w. In some embodiments, the plant-based honey composition comprises sea buckthorn extract in an amount between at least about 0.0001% w/w, about 0.0005% w/w, about 0.001% w/w, about 0.0015% w/w, about 0.002% w/w, about 0.005% w/w, about 0.01% w/w, about 0.05% w/w, about 0.1% w/w, about 0.4% w/w, or about 0.8% w/w. In some embodiments, the plant-based honey composition comprises sea buckthorn extract in an amount between at most about 0.0005% w/w, about 0.001% w/w, about 0.0015% w/w, about 0.002% w/w, about 0.005% w/w, about 0.01% w/w, about 0.05% w/w, about 0.1% w/w, about 0.4% w/w, about 0.8% w/w, or about 1% w/w. In some embodiments, the plant-based honey formulation may comprise sea buckthorn extract in an amount of from 0.001% to about 1% w/w of the formulation. In some embodiments, the plant-based honey formulation may comprise sea buckthorn extract in an amount of from 0.01% to about 1% w/w of the formulation. In some embodiments, the plant-based honey formulation may comprise sea buckthorn extract in an amount of from 0.10% to about 10% w/w of the formulation. In some embodiments, the plant-based honey formulation may comprise sea buckthorn extract in an amount from 0.05% to about 0.5% w/w. The non-bee made honey formulation may comprise sea buckthorn extract in an amount of about 0.10% w/w.

A second mixture component of the formulation may comprise a set of additional ingredients added to the first mixture of ingredients in the plant-based non-bee honey composition to impart a unique profile. These additional ingredients may include those that may vary considerably between honeys based on floral origin, and that may also contain volatile compounds that interact strongly with olfactory and taste receptors to give the consumer of honey a distinct sensory experience, for example, to produce a non-bee made clover honey product.

As noted in the Codex alimentarius, set forth by the Food and Agriculture Organization of the United Nations (FAO) and World Health Organization (WHO), a great number of different compounds contribute to honey including organic acids, alcohols, aldehydes, and esters. The later compounds belong to a larger family known as volatile organic compounds (VOCs), many of which impart strong aromas and flavors mixtures.

Upwards of 600 volatile organic compounds (VOCs) have been identified in many combinations in honeys [Manyi-Loh Int J Mol Sci. 12(12):9514-9532 (2011); Montenegro. Int. J. Exp. Bot. 78:61-65 (2009)]. These compounds originate from a variety of biosynthetic pathways belonging to the plant(s) providing the floral source of the nectar, bees, and even those of commensal microorganisms. Chemical families to which these many volatile organic compounds belong include those described in the following publications: Manyi-Loh 2011; Barra, Chile. Chilean J. Agric. Res. 70:75-84 (2010); Escriche. Food Chem. 1; 226:61-68 (2017).

Representative compounds include hydrocarbons, aldehydes, alcohols, ketones, acids, esters, benzene and derivatives, furan and pyran, norisoprenoids, terpenes and derivatives, sulphur-containing compounds, nitrogen compounds, and cyclic compounds.

VOCs belonging to one, several, or all classes can be mixed together to create unique VOC flavor/scent compositions approaching that of honey. These compositions can then be added to the honey base to achieve the flavor/scent sensory experience expected of a given honey variety.

### B. Physical Characteristics:

### Moisture Content

Water content, as measured by refractometry, may be within a range of 5-40%. Water content may be in a range of about 10-40%. Water content may be in a range of about 25-35%. Water content may be in a range of about 32%. Water content may be in a range of about 15% in a low moisture formulation. Water activity (aw) may be <0.65 in the final honey base. Water activity (aw) may range from about 0.4-0.7. Water activity (aw) may be about 0.5. In some embodiments, the non-bee made honey formulation comprises a water activity of about 0.4 to about 0.6. In some embodiments, the non-bee made honey formulation comprises a water activity of about 0.5. In some embodiments, the non-bee made honey formulation comprises a water activity of about 0.45 to about 0.6. In some embodiments, the non-bee made honey formulation comprises a water activity of about 0.5. In some embodiments, the non-bee made honey composition has a water activity of about 0.4. In some embodiments, the non-bee made honey composition has a water activity of about 0.5. In some embodiments, the non-bee made honey composition has a water activity of about 0.45. In some embodiments, the non-bee made honey composition has a water activity of between about 0.4 and 0.5. In some embodiments, the non-bee made honey composition has a water activity of between about 0.35 and 0.5. In some embodiments, the non-bee made honey composition has a water activity of between about 0.35 and 0.55.

### Density

Density is the ratio between weight of the honey and volume at a given temperature. The density of the plant honey base weight/volume may be between 1.35 and 1.50 at 20° C. A liter of honey may weigh between 1350 and 1500 grams. In some embodiments, the honey may weigh between 1400-1450 grams per 1 L at 20 C.

### Brix Value

Brix value is a measure of how much dissolved sugar is in a liquid solution. This value is indicated in degrees Brix. One degree of brix means that a hundred grams of liquid solution contains one gram of sugar. The formulations disclosed herein may comprise a Brix value from about 70 to about 90, about 75 to about 85, and about 81.5 Brix.

### Viscosity

In some embodiments, the non-bee made honey formulation comprises a viscosity of about 14,000 cP to about 17,000 cP. In some embodiments, the non-bee made honey formulation comprises a viscosity of about 14,000 cP to about 16,000 cP. In some embodiments, the non-bee made honey formulation comprises a viscosity of about 15,000 cP to about 17,000 cP. In some embodiments, the non-bee made honey comprises a viscosity of about 16,000 cP.

### III Methods of Making

In another aspect, provided herein is a method of producing the non-bee made honey formulation disclosed herein, comprising: mixing glucose and fructose in water under elevated temperature to form a first solution; mixing one or more organic acids, and one or more polyphenols in water under elevated temperature to form a second solution; and mixing the first solution and the second solution to form a third solution; and dehumidifying the third solution at elevated temperature while mixing to reduce the water content of the solution.

Plant honey base creation may include the following steps:
(1) Dissolution of sugars in water under light heating to form a first solution;
(2) Dissolution of acids and/or polyphenols, and other ingredients in water to form second solution;
(3) Mixing the first and second solution to form a third solution;
(4) Dehumidification; and, optionally,
(5) Storage for later use.

### A. Creation of the Non-Bee Honey

The sugar solution may be produced by combining the glucose, fructose, and optionally one or more of the organic acids under light heating, and optional stirring. Thereafter, the polyphenol and extract blends are added as an aqueous mixture to the dissolved sugar solution. Then, the solution may be dehumidified to the desired water concentration.

The flavor of the formulation comprising the sugars and organic acids may be to be sweet, and acidic with little or no aftertaste and little or no scent/aroma; with a balance flavor of sweetness and acidity to emulate the sensory experience expected from bee-made clover honey. The base of the clover honey formation may have a neutral character, able to be built upon by specific top-notes, for example, by polyphenols contributing light floral notes.

The goal of the formulation is to provide a foundational taste similar to honey, with a mix of sweet and acidic characteristics. However, the base should have little to no smell. By balancing these flavors, the resulting sensory experience will mirror that of bee-made honey.

The neutral character of the base can be built upon by specific additional ingredients to emulate specific types of honey. The additional ingredients can comprise a variety of other additional polyphenols, acids, flavorings, fragrances, and volatile compounds to perfect the bee-free honey formulation.

Other goals and advantages of the invention will be further appreciated and understood when considered in conjunction with the following description and accompanying drawings. While the following description may contain specific details describing particular embodiments of the invention, this should not be construed as limitations to the scope of the invention but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### EXAMPLES

### Example 1: Preparation of Non-Bee Made Honey Composition Formula B

In this example, a clover honey composition is prepared which is intended to mimic the taste, texture, color, and other properties of clover honey. The non-bee honey composition is prepared by heating a solution of purified water to about 50 C while constantly stirring the solution, and adding fructose and glucose in amounts to produce a composition of about 39% w/w fructose, about 28% w/w glucose, and about 33% water, to produce a sugar base solution to approximate the sweetness of natural clover honey. Next, organic acids in amounts to produce a final dehydrated composition of about 0.5% w/w gluconic acid (at 99%+ purity or 0.1% a 50% purity) and 0.01% succinic acid are added to the composition to provide acidity necessary to approximate the flavor of natural clover honey, as well as aid in the dissolution of the sugars. Then the composition is dehydrated to a water content of about 17% w/w water, and forms a supersaturated sugar solution of about 49% w/w fructose, and about 35% w/w glucose. The composition is then transferred to individual containers and cooled to complete the non-bee honey composition.

When evaluated in a sensory panel against bee-made clover honey as described in Example 4; this Formulation B honey is found to be mostly clear in appearance, not look like bee-made honey, to have a viscosity and texture which is different from regular honey, lack the aroma of regular honey, and was otherwise found to be an unsatisfactory substitute to bee-made honey as measured by sensory attributes and likability of the same as compared to bee-made honey. It is unexpected and surprising that the composition of Formulation B would rate so differently from bee-made clover honey considering that it is highly similar to bee-made clover honey by mass percentage of sugars, water, and acids.

### Example 2: Preparation of Non-Bee Made Honey Composition Formula A

In this example, a clover honey composition is prepared which is intended to mimic the taste, texture, color, and other properties of clover honey. The non-bee honey composition is prepared by heating a solution of purified water to about 50 C while constantly stirring the solution, and adding fructose and glucose in amounts to produce a composition of about 39% w/w fructose, about 28% w/w glucose, and about 33% water, a sugar base solution to approximate the sweetness of natural clover honey. Next, organic acids in amounts to produce a composition of about 0.5% w/w gluconic acid (at 99%+ purity or 0.1% a 50% purity), 0.01% succinic acid, 0.0042% chlorogenic acid, and 0.0017% gallic acid are added to the composition to provide acidity necessary to approximate the flavor of natural clover honey, as well as aid in the dissolution of the sugars and subsequently added polyphenols. Then, polyphenols in amounts to produce a final dehydrated composition of about 0.003% apigenin, and about 0.005% chrysin are added to the composition to provide a distinct flavor profile, aroma, and color which approximates natural clover honey. Then the composition is dehydrated to a water content of about 17% w/w water, and forms a supersaturated sugar solution of about 49% w/w fructose, about 35% w/w glucose, and with the above stated polyphenol and acid content. The composition is then transferred to individual containers and cooled to complete the non-bee honey composition.

As compared to Formula B of Example 1, this formulation comprises the polyphenols of about 0.003% apigenin, and about 0.005% chrysin, and organic acids of 0.0042% chlorogenic acid, and 0.0017% gallic acid.

When evaluated in a sensory panel against bee-made clover honey as set forth in Example 4; this Formulation A honey is found to be mostly clear in appearance, not look like bee-made honey, to have a viscosity and texture which is different from regular honey, lack the aroma of regular honey, and was otherwise found to be an unsatisfactory substitute to bee-made honey as measured by sensory attributes and likability of the same as compared to bee-made honey. It is surprising the formulation of Example 2 disclosed herein would rate so differently from bee-made clover honey considering that it is highly similar to bee-made clover honey by mass percentage of sugars, water, acids, and some polyphenols.

Notwithstanding the overall unsatisfactory product described this example; it is noted that Formula A of Example 2 herein rates higher in nearly all attributes and likability in the comparison to bee-made clover honey as compared to Formula B of Example 1, indicating that the addition of the polyphenols of about 0.003% apigenin, and about 0.005% chrysin, and organic acids of 0.0042% chlorogenic acid, and 0.0017% gallic acid did significantly improve the clover honey like properties of the formulation.

### Example 3: Preparation of MeliBio Non-Bee Made Honey Composition

In this example, a clover honey composition is prepared which mimics the taste, texture, color, and other properties of clover honey. The non-bee honey composition is prepared by heating a solution of purified water to about 155 F while constantly stirring the solution, and adding fructose and glucose in amounts to produce a composition of about 39% w/w fructose, about 28% w/w glucose, and about 33% water, to produce a sugar base solution to approximate the sweetness of natural clover honey. Next, organic acids in amounts to produce a final dehydrated composition of about 0.5% w/w gluconic acid (at 99%+ purity or 0.1% a 50% purity), 0.01% succinic acid, 0.0042% chlorogenic acid, and 0.005% gallic acid are added to the composition to provide acidity necessary to approximate the flavor of natural clover honey, as well as aid in the dissolution of the sugars and subsequently added polyphenols. Then, polyphenols in amounts to produce a final dehydrated composition of about 0.01% quercetin, 0.005 chrysin, 0.003% apigenin, and 0.1% sea buckthorn extract are added to the composition to provide a unique flavor profile, aroma, and color which approximates natural clover honey. Sea buckthorn extract as utilized herein comprises proanthocyanidins and the polyphenols kaempferol, quercetin, and isorhamnetin. Then the composition is dehydrated to a water content of about 17% w/w water, and forms a supersaturated sugar solution of about 49% w/w fructose, and about 35% w/w glucose. The composition is then transferred to individual containers and cooled to complete the MeliBio non-bee made honey composition.

When evaluated in a sensory panel against bee-made clover honey as set forth in Example 4; this MeliBio clover honey formulation of Example 3 is found to be golden-yellow in appearance, look like bee-made honey, to have a viscosity and mouthfeel which is like regular honey, to smell like bee-made honey, and was generally found to be a satisfactory substitute to bee-made honey as measured by sensory attributes and likability of the same as compared to bee-made honey.

When considering the same data as applied to Formula B of Example 1, and Formula A of Example 2; it is noted that the instant MeliBio clover honey formulation of Example 3 herein rates higher in nearly all attributes and likability in the comparison to bee-made clover honey, indicating that the addition of the polyphenols of about 0.003% apigenin, about 0.005% chrysin, 0.01% quercetin, and 0.1% sea buckthorn extract, and organic acids of about 0.5% w/w gluconic acid (at 99%+ purity or 0.1% a 50% purity), 0.01% succinic acid, 0.0042% chlorogenic acid, and 0.005% gallic acid did significantly improve the clover honey like properties of the formulation. It is further surprising that the additional of the polyphenols of about, 0.01% quercetin, and 0.1% sea buckthorn extract, and organic acids 0.0042% chlorogenic acid, and 0.005% gallic acid significantly improved the nearly all attributes and likability in the comparison to bee-made clover honey as compared to Formula A of Example 2.

### Example 4: Sensory Panel Evaluating Formula A, Formula B, MeliBio Formula, and Natural Clover Honey

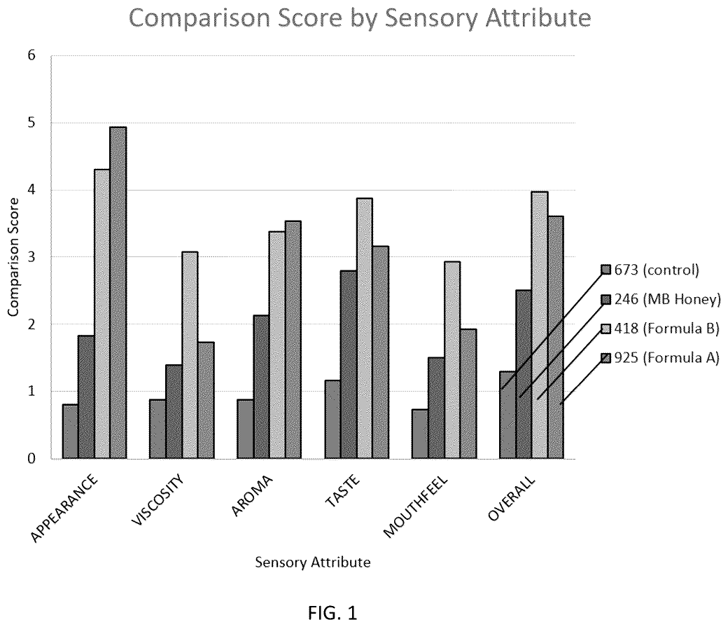

In this example, multiple non-bee honey formulations are compared with bee-derived clover honey in a sensory panel conducted by an independent third party. In this example, 29 participants that consumed honey at least once per week were given samples to consume of each formulation and are asked to evaluate multiple factors of the samples including appearance, viscosity, mouth-feel, aroma, taste, and an overall similarity factor. The formulations tested are those as described in Examples 1-3 above. These parameters are rated on a 0 to 5 scale with the following description. The data of the overall comparison properties of the various honey formulations is shown in FIG. 1
5: There is no resemblance between the two products.
4: There is a striking or clear difference between the properties
3: There is a noticeable difference between the properties.
2: The non-bee honey composition is very close to natural clover honey, but a minor difference can be detected upon careful inspection.
1: The non-bee honey composition is indistinguishable from natural clover honey.

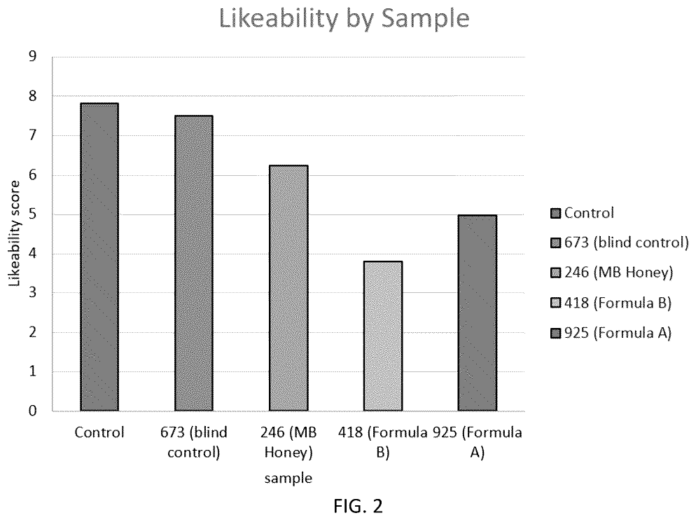

In addition, there is a likeability parameter which characterizes the overall appeal of the formulation. The parameter is rated on a 1 to 9 scale with the following descriptions. The data of the overall likability of the various honey formulations is shown in FIG. 2.
1: Dislike extremely
2: Dislike very much
3: Dislike moderately
4: Dislike slightly
5: Neither like nor dislike
6: Like slightly
7: Like moderately
8: Like very much
9: Like extremely

Data suggests that MeliBio honey of Example 3 compared very well and closely approximated the control natural bee-made honey. In this example, the natural bee-made honey was a clover honey product produced in the United States, Safeway Signature Select brand. There was also a clear increase in closeness to control and overall likeability of the final formulation honey that included the key polyphenols quercetin and kaempferol, isorhamnetin, and proanthocyanidins (provided by purified Sea Buckthorn extract). The earlier formulations that lacked these components (e.g., Examples 1 and Example 2) did not score as highly across nearly all attributes (see FIGS. 1-2).

This survey enables a key comparison between non-bee honey and normal bee-derived clover honey. The results demonstrate a clear distinguishment between non-bee made honey composition A (Example 2), composition B (Example 1), and MeliBio honey across all surveyed parameters. In particular, bee-derived honey and MeliBio honey had much higher likeability scores whereas non-bee made honey formulations A and B did not demonstrate likeability scores of about 4.5 on average. On the other hand, MeliBio formulation demonstrated an average likeability of above 6. In terms of other surveyed parameters, the MeliBio formulation was rated more similarly to bee-derived clover honey than formulations A and B across all surveyed parameters. There was a particularly close rating for appearance between MeliBio non-bee honey and bee-derived clover honey.

A key result that emerged is the key roles both organic acids and polyphenols play in likeability and similarity in various aspects of the honey composition such as appearance, flavor, aroma, color, and mouthfeel, notwithstanding the fact that they constitute less than 1% of the overall formulation by weight. The non-bee made honey formulations A and B yielded unfavorable surveys as a honey substitute as a result of missing the lack of polyphenols quercetin and proanthocyanidins in Example 3, even though these components measured significantly less than 1% w/w of the overall formulation. Similarly, the lack of organic acids gallic acid and chlorogenic acid in non-bee made honey formulations A and B resulted in unfavorable surveys as a honey substitute, even though these components measured significantly less than 1% w/w of the overall formulation.

It is unexpected that the flavor, texture, appearance, and other properties of natural clover honey could be imitated by the MeliBio non-bee made honey of Example 3, as indicated in the above trial, and could be approximated with the disclosed combination and concentration of organic acids and polyphenols, in the absence of the specific polyphenols, pollen, enzymes, proteins, bee venom, and other components present in natural bee-made honey. For example, it is surprising and unexpected that the non-bee made honey composition described herein in Example 3 which comprises the polyphenols apigenin, chrysin, quercetin, sea buckthorn extract, and the organic acids chlorogenic acid, gallic acid, and succinic acid, and gluconic acid present at less than 1% by mass of the overall composition would be important ingredients in producing a non-bee made honey formulation which closely mimics bee-made clover honey.

### Example 5: Exemplary MeliBio Product Analysis for Phenolic Content

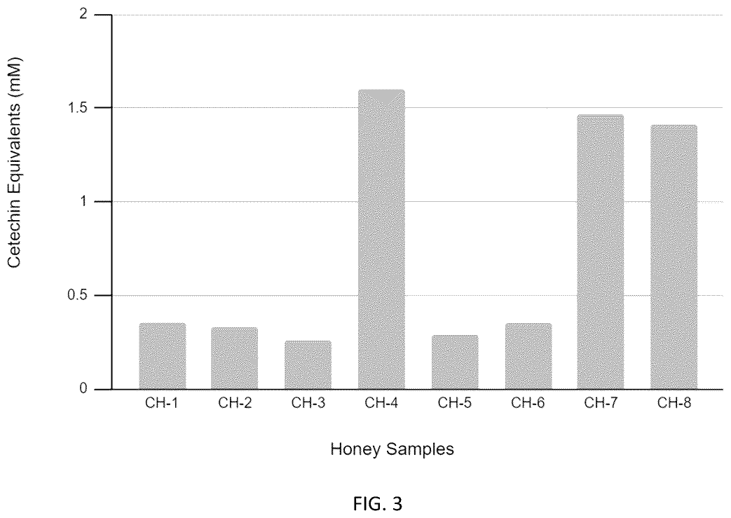

In this example, the amount of phenolic compounds within MeliBio honey formulations as prepared in Examples 1-3 was analyzed using a catechin equivalent assay in order to quantify the difference in total phenolic content between natural bee made honey and the MeliBio formulations described in Examples 1-3 herein. The results of the catechin equivalent assay are shown in FIG. 3, and illustrates that the concentration of phenolic compounds was unexpectedly found to be significantly higher in MeliBio honey formulations as prepared in Examples 1-3 as opposed to natural bee-made honey.

TABLE 2

| Sample | Catechin equivalents (mM) | STDEV |
|---|---|---|
| CH-1 | 0.3512987013 | 0.002687005769 |
| CH-2 | 0.3298701299 | 0.007353910524 |
| CH-3 | 0.2609307359 | 0.0007778174593 |
| CH-4 | 1.602597403 | 0.02531442277 |
| CH-5 | 0.2917748918 | 0.007848885271 |
| CH-6 | 0.3501082251 | 0.008980256121 |
| CH-7 | 1.467099567 | 0.02602152955 |
| CH-8 | 1.412662338 | 0.03669884194 |

TABLE 3

| Sample Code | Sample Identity |
|---|---|
| CH-1 | Signature Select |
| CH-2 | Sue Bee |
| CH-3 | BuzzBees |
| CH-4 | Formula A, Example 2 |
| CH-5 | Glory Bee |
| CH-6 | Chico Honey Co |
| CH-7 | Formula B, Example 1 |
| CH-8 | MeliBio Honey, Example 3 |

### Example 6: Exemplary MeliBio Product Analysis for Protein Content

In this example, the amount of protein within MeliBio honey formulations as prepared in Examples 1-3 was analyzed using a AOAC 2001.11 assay in order to quantify the difference in total protein content between natural bee made honey and the MeliBio formulations described in Examples 1-3 herein. The results of the AOAC 2001.11 assay in the below Table 4, and illustrates that the concentration of protein was found to be significantly higher in natural bee made honey as opposed to the formulations as prepared in Examples 1-3. Protein content in honey may be the result of allergens, bee parts, pollens, other organic proteins which could serve as potential allergens to consumers of honey, or spores of botulin toxin which render honey unsuitable for consumption by infants. The lack of any detectable protein in the honey in the formulations as prepared in Examples 1-3 indicates the samples are free or substantial free of allergens, bee parts, pollens, other organic proteins which could serve as potential allergens to consumers. The lower detectable limit for protein in this assay was 0.1 g/100 g.

TABLE 4

| Sample Name | Protein (g/100 g) |
|---|---|
| CH-1 | 0.23 |
| CH-2 | 0.22 |
| CH-3 | 0.17 |
| CH-4 | <LOQ |
| CH-5 | 0.25 |
| CH-6 | 0.22 |
| CH-7 | <LOQ |
| CH-8 | <LOQ |

### Example 7: Exemplary Non-Bee Made Honey Composition

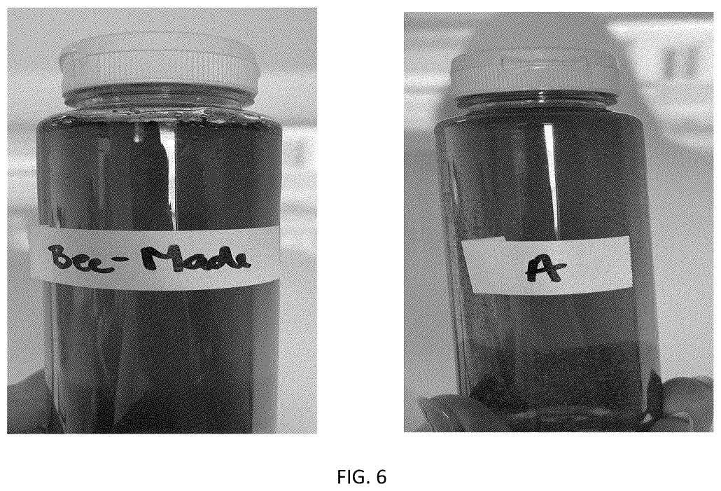

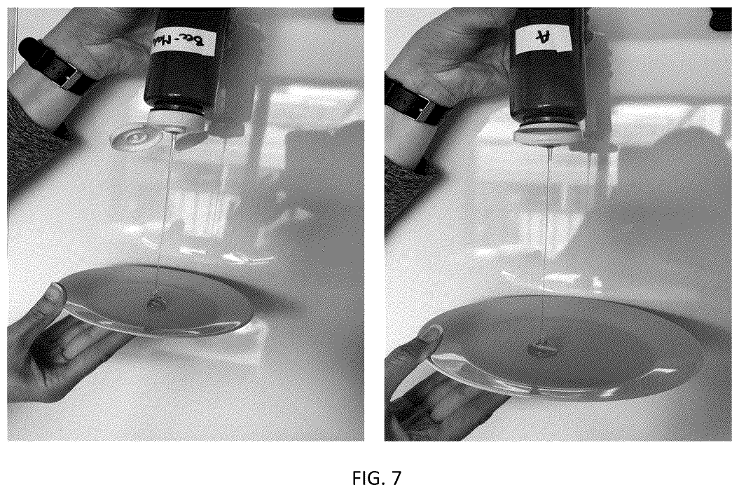

In this example, the components of and weight percent of components for exemplary non-bee made honey formulations are disclosed. Different compositional ensembles can yield distinct flavor and nutritional aspects to non-bee made honey, and disclosed herein is a non-bee made clover honey formulation. The first formulation is shown in Table 5 and is pictured as the composition labeled “A” in FIGS. 6-7, which illustrate the non-bee made clover honey formulation closely mirror the appearance, texture, and cohesive properties of bee-made clover honey.

TABLE 5

| Ingredient | wt./wt. % |
|---|---|
| Crystalline fructose powder | 40-60% |
| Dextrose monohydrate | 20-40% |
| Water | 12-20% |
| Gluconic acid | 0.05-0.20% |
| Sea buckthorn | 0.05-0.5% |
| Chamomile | 0.001-0.05% |
| Red Clover | 0.001-0.05% |
| Jasmine | 0.001-0.05% |
| Lavender | 0.001-0.05% |
| Passionflower | 0.001-0.05% |
| Succinic acid | 0.05-0.1% |

### Example 8: Exemplary Non-Bee Made Honey Formulations, Total Antioxidant Content, and Phenolic Content

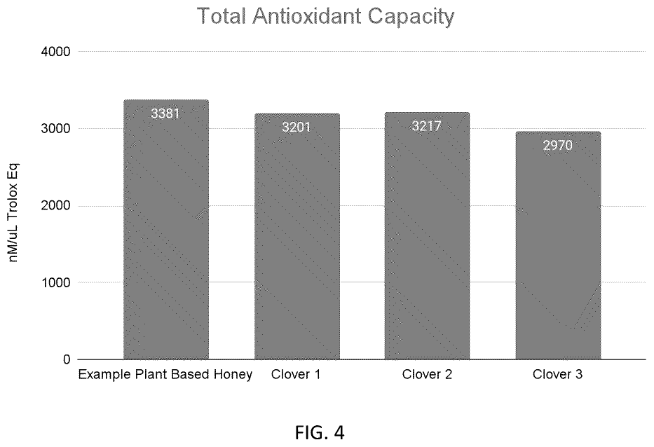

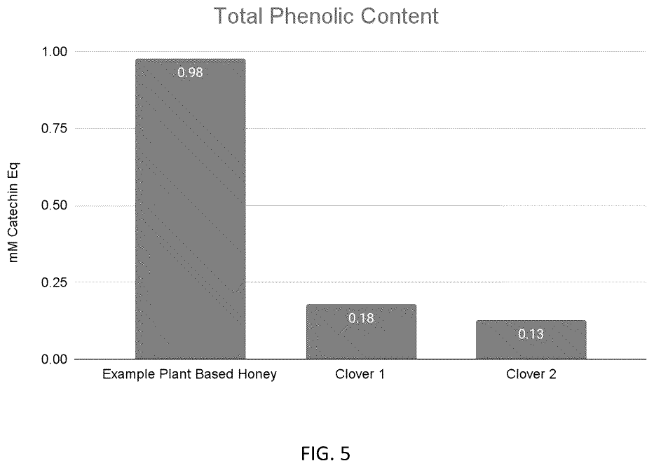

In this example, of components for exemplary non-bee made honey formulations was disclosed and was assessed for total antioxidant content, and total phenolic content. The components for the formulation labeled Example Plant Based Honey and illustrated in FIGS. 6-7 are shown in Table 8 below, and were assayed for total antioxidant content, and total phenolic content using methods known with the art. The non-bee made honey formulation was assayed for total antioxidant content via a Trolox equivalent assay measured with Sigma MAK187, and was assayed for total phenolic content by a catechin equivalent assay with Sigma MAK365. The total antioxidant capacity measured by a Trolox equivalent assay is shown in FIG. 4, and was compared to natural bee-mode clover honey. The concentration of phenolic compounds is shown in FIG. 5, and was compared to natural bee-mode clover honey. It is shown that the total antioxidant activity of the non-bee made honey formulation was at least that of natural bee made honey (FIG. 4), and that the concentration of phenolic compounds significantly excessed that natural bee-mode clover honey (FIG. 5). Notwithstanding the high total antioxidant activity and high phenolic compound concentrations, the non-bee made honey formulation closely approximates the taste, appearance, color, texture, and cohesiveness of a natural clover honey (FIGS. 6-7).

TABLE 8

| Ingredient | wt./wt. % |
|---|---|
| Crystalline fructose powder | 40-60% |
| Dextrose monohydrate | 20-40% |
| Water | 12-20% |
| Gallic acid | 0.001-0.05% |
| Succinic acid | 0.05-0.1% |
| Sea buckthorn powder | 0.05-0.5% |
| Gluconic acid | 0.05-0.20% |

### Example 9: Exemplary Non-Bee Made Honey Formulations, Total Antioxidant Content, and Phenolic Content

In this example, the exemplary non-bee made honey formulation of Example 7 is assessed for total antioxidant content, and total phenolic content, as was done in Example 8. The non-bee made honey formulation is assayed for total antioxidant content via a Trolox equivalent assay measured with Sigma MAK187, and is assayed for total phenolic content by a catechin equivalent assay with Sigma MAK365. It is shown that the total antioxidant activity of the non-bee made honey formulation is at least that of natural bee made honey, and that the concentration of phenolic compounds significantly excessed that natural bee-mode clover honey. Notwithstanding the high total antioxidant activity and high phenolic compound concentrations, the non-bee made honey formulation closely approximates the taste, appearance, color, texture, and cohesiveness of a natural clover honey.

We claim:

1. A non-bee made honey formulation comprising:
a. fructose in an amount from about 20% to about 55% w/w;
b. glucose in an amount from about 20% to about 40% w/w;
c. one or organic acids in an amount from about 0.001% to about 5% w/w;
d. water; and
e. one of the following:
i. one or more polyphenols in a concentration of at least 0.4 mM Catechin Equivalents; or
ii. a total antioxidant capacity of at least 1,500 nM/uL Trolox Equivalents.

2. The non-bee made honey composition of claim 1, wherein the total antioxidant capacity is at least that of natural bee-made clover honey.

3. The non-bee made honey composition of claim 1, wherein the total antioxidant capacity exceeds that of natural bee-made clover honey.

4. The non-bee made honey composition of claim 1, wherein the total antioxidant capacity exceeds that of natural bee-made clover honey by at least 2.5% wt.

5. The non-bee made honey composition of claim 1, wherein the total antioxidant capacity is at least 2,500 nM/uL Trolox Equivalents.

6-11. (canceled)

12. The non-bee made honey composition of claim 1, further comprising one or more extracts are selected from: Sea buckthorn, Chamomile, Red Clover, Jasmine, Lavender, Succinic acid, or Passionflower.

13. The non-bee made honey composition of claim 1, further comprising Sea buckthorn extract, and one or more extracts selected from: Chamomile, Red Clover, Jasmine, Lavender, Succinic acid, or Passionflower.

14. The non-bee made honey composition of claim 1, further comprising Sea buckthorn extract, and three or more extracts selected from: Chamomile, Red Clover, Jasmine, Lavender, Succinic acid, or Passionflower.

15. The non-bee made honey composition of claim 1, further comprising Sea buckthorn extract, Chamomile extract, Red Clover, and Succinic acid.

16. The non-bee made honey composition of claim 1, further comprising one or more extracts comprising: Sea buckthorn, Reishi mushroom, Chaga mushroom, Green coffee bean, Ginger, Pu'erh tea, Matcha, Acai berry, Aronia berry, Hibiscus, Squash blossom, Chamomile, Cinnamon, Orange blossom, Green tea, Lemon or lemon juice, white tea, grape seed, or Shilajit (mineral powder).

17. The non-bee made honey composition of claim 1, further comprising at least two extracts selected from: Sea buckthorn, Reishi mushroom, Chaga mushroom, Green coffee bean, Ginger, Pu'erh tea, Matcha, Acai berry, Aronia berry, Hibiscus, Squash blossom, Chamomile, Cinnamon, Orange blossom, Green tea, Lemon, white tea, grape seed, or Shilajit (mineral powder).

18-28. (canceled)

29. The non-bee made honey formulation of claim 1, wherein the formulation comprises one or more proanthocyanidins in an amount from 0.00010% to about 0.10% w/w.

30. The non-bee made honey formulation of claim 1, wherein the formulation comprises one or more proanthocyanidins in an amount up to about 0.2 mg per 30 mL of the formulation.

31. The non-bee made honey formulation of claim 1, wherein the formulation comprises one or more proanthocyanidins in an amount of at least 0.0001%.

32. The non-bee made honey formulation of claim 1, wherein the formulation comprises one or more proanthocyanidins in an amount of 0.0001% to about 0.02% w/w.

33-37. (canceled)

38. The non-bee made honey formulation of claim 1, wherein the one or more polyphenols comprise quercetin in an amount greater that 0.0015% w/w.

39. The non-bee made honey formulation of claim 1, wherein the formulation comprises quercetin in an amount from about 0.0015% to about 0.1% w/w.

40. The non-bee made honey formulation of claim 1, wherein the formulation comprises quercetin in an amount of about 0.003% to about 0.005% w/w.

41. The non-bee made honey formulation of claim 1, wherein one of the one or more organic acids comprise gluconic acid in an amount from about 0.001% to about 0.5% w/w.

42-76. (canceled)

77. The non-bee made honey formulation of claim 1, further comprising sea buckthorn extract in an amount from 0.05% to about 0.5% w/w
